SEQUENCE LISTING (70 sequences)

SEQ ID NO: 1 - DNA, 645 bp, Homo sapiens
  misc_feature: Full length NKG2D
gggtggattcgtggtcggaggtctcgacacagctgggagatgagtgaatttcataattataacttggatctgaagaagagtgatttttcaacacgatggcaaaagcaaagatgtccagtagtcaaaagcaaatgtagagaaaatgcatctccattttttttctgctgcttcatcgctgtagccatgggaatccgtttcattattatggtaacaatatggagtgctgtattcctaaactcattattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgcatgcaaaggactgtg

SEQ ID NO: 2 - DNA, 405 bp, Homo sapiens
  misc_feature: Truncated NKG2D
ttattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgcatgcaaaggactgtg

SEQ ID NO: 3 - DNA, 405 bp, Homo sapiens
  misc_feature: Codon Optimized Truncated NKG2D
ctgttcaatcaggaagtccagatccccctgacagagtcttactgcggcccatgtcccaagaactggatctgctacaagaacaattgttatcagttctttgacgagagcaagaactggtatgagtcccaggcctcttgcatgagccagaatgcctctctgctgaaggtgtacagcaaggaggaccaggatctgctgaagctggtgaagtcctatcactggatgggcctggtgcacatccctacaaacggctcttggcagtgggaggacggctccatcctgtctccaaatctgctgaccatcatcgagatgcagaagggcgattgcgccctgtacgccagctccttcaagggctatatcgagaactgctccacacccaatacctacatctgtatgcagaggaccgtg

SEQ ID NO: 4 - DNA, 63 bp, Homo sapiens
  misc_feature: CD8 Signaling Sequence
atggctctgcccgtcaccgcactgctgctgcctctggctctgctgctgcacgccgcacgacca

SEQ ID NO: 5 - DNA, 135 bp, Homo sapiens
  misc_feature: CD8 alpha hinge
accacaacccctgcaccacgcccccctacaccagcacctaccatcgcaagccagcctctgtccctgcggccagaggcatgtagaccagcagcaggaggagcagtgcacacaagaggcctggacttcgcctgcgat

SEQ ID NO: 6 - DNA, 1722 bp, Homo sapiens
  misc_feature: CD8 beta
  misc_feature (674)..(773): n is a, c, g, or t
  misc_feature (859)..(958): n is a, c, g, or t
atctaggtcttgctgcacccgcacaacctacaaacagcgtcggggccttctctgcacctccagttcccagctcacctccctcagtgtcacagccggttacctttccttcctccctgggggagggcaagacttggggcttgctgactccaggcccagcccagcccggggcacccaggagcccctcaattgctactcaaacagacaagaagcggcccgagttagtggccagctccaccatgcactacacatcctgacctctctgagcctctactgtcactcggggtcacaaccctttcctgagcacctcccggggcagggggcgatgacacacatgcagctgcctgggggaggccggcggtgtcccctcctttctggaacgcggagggtcctggtgggctctggaaacgcagcccagacctttgcaatgctaggaggatgagggcggagacctcgcggtccccaacaccagactcccgcagccaccgcgcccggtcccgccctccccactgcccccccagctccccgacccaggcgccccgcccggccagctcctcacccaccccagccgcgactgtctccgccgagcccccggggccaggtgtcccgggcgcgccacgatgcggccgcggctgtggctcctcctggccgcgcagctgacaggtaaggcggcggcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnttgctttcctcttccaggccggcggaggagagcccggcttcgtttcatgaaacagtaagtgtataacctgggtgtggccttgggannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncttgctgttgttttcagattttacaaatgagcagagaatacggttttggtgtcctgctacaaaaagacatcggtcagtaacgagcacgatgtggaaaaatgagagaagggacacattcaaccctggagagttcaatggctgctgaagctgcctgcttttcactgctgcaaggcctttctgtgtgtgacgtgcatgggagcaacttgttcgtgggtcatcgggaatactagggagaaggtttcattgcccccagggcacttcacagagtgtgctggaggactgagtaagaaatgctgcccatgccaccgcttccggctcctgtgctttccctgaactgggacctttagtggtggccatttagccaccatctttgcaggttgctttgccctggtagggcagtaacattgggtcctgggtctttcatggggtgatgctgggctggctccctgttggtcttcccaggctggggctgaccttcctcgcagagaggccaggtgcaggttgggaatgaggcttgctgagaggggctgtccagttcccagaaggcatatcagtctctgagggcttcctttggggccgggaacttgcgggtttgaggataggagttcacttcatcttctcagctcccatttctactcttaagtttctcagctcccatttctactctcccatggcttaatgcttctttcattttctgtttgttttatacaaatgtcttagttgtaaaaataaagtcccaggttaaagataacaaacgggtcctg

SEQ ID NO: 7 - DNA, 2415 bp, Homo sapiens
  misc_feature: CD16 alpha
attcttggtgctgggtggatccaaatccaggagatggggcaagcatcctgggatggctgagggcacactctggcagattctgtgtgtgtcctcagatgctcagccacagacctttgagggagtaaagggggcagacccacccaccttgcctccaggctctttccttcctggtcctgttctatggtggggctcccttgccagacttcagactgagaagtcagatgaagtttcaagaaaaggaaattggtgggtgacagagatgggtggaggggctggggaaaggctgtttacttcctcctgtctagtcggtttggtccctttagggctccggatatctttggtgacttgtccactccagtgtggcatcatgtggcagctgctcctcccaactgctctgctacttctagtttcagctggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagtcgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggctttttgggagtaaaaatgtgtcttcagagactgtgaacatcaccatcactcaaggtttggcagtgtcaaccatctcatcattctttccacctgggtaccaagtctctttctgcttggtgatggtactcctttttgcagtggacacaggactatatttctctgtgaagacaaacattcgaagctcaacaagagactggaaggaccataaatttaaatggagaaaggaccctcaagacaaatgacccccatcccatgggggtaataagagcagtagcagcagcatctctgaacatttctctggatttgcaaccccatcatcctcaggcctctctacaagcagcaggaaacatagaactcagagccagatcccttatccaactctcgacttttccttggtctccagtggaagggaaaagcccatgatcttcaagcagggaagccccagtgagtagctgcattcctagaaattgaagtttcagagctacacaaacactttttctgtcccaaccgttccctcacagcaaagcaacaatacaggctagggatggtaatcctttaaacatacaaaaattgctcgtgttataaattacccagtttagaggggaaaaaaaaacaattattcctaaataaatggataagtagaattaatggttgaggcaggaccatacagagtgtgggaactgctggggatctagggaattcagtgggaccaatgaaagcatggctgagaaatagcaggtagtccaggatagtctaagggaggtgttcccatctgagcccagagataagggtgtcttcctagaacattagccgtagtggaattaacaggaaatcatgagggtgacgtagaattgagtcttccaggggactctatcagaactggaccatctccaagtatataacgatgagtcctcttaatgctaggagtagaaaatggtcctaggaaggggactgaggattgcggtggggggtggggtggaaaagaaagtacagaacaaaccctgtgtcactgtcccaagttgctaagtgaacagaactatctcagcatcagaatgagaaagcctgagaagaaagaaccaaccacaagcacacaggaaggaaagcgcaggaggtgaaaatgctttcttggccagggtagtaagaattagaggttaatgcagggactgtaaaaccaccttttctgcttcaatatctaattcctgtgtagctttgttcattgcatttattaaacaaatgttgtataaccaatactaaatgtactactgagcttcgctgagttaagttatgaaactttcaaatccttcatcatgtcagttccaatgaggtggggatggagaagacaattgttgcttatgaaagaaagctttagctgtctctgttttgtaagctttaagcgcaacatttcttggttccaataaagcattttacaagatcttgcatgctactcttagatagaagatgggaaaaccatggtaataaaatatgaatgataaaaaaaaaaaaaaaaaaaaaaaaaaaaaaaaaa

SEQ ID NO: 8 - DNA, 2473 bp, Homo sapiens
  misc_feature: CD16 beta
  misc_feature (211)..(310): n is a, c, g, or t
  misc_feature (537)..(636): n is a, c, g, or t
  misc_feature (968)..(1067): n is a, c, g, or t
aaagatgggtggagggactggggaaaggctgtttactccctcctgtctagtcggcttggtccctttaggggtccggatatctttggtgacttgtccactccagtgtggcatcatgtggcagctgctcctcccaactgctctgctacttctaggtaagtaggatctccctggttgagggagaagtttgagatgccttgggttcagcagagannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaagaggcatgaacagtggaagaccagagagcaggtagcaaggtttccaccagaaacatcctgattcttgggaaaattgggctcctggggcagaggagggcaggggagttttaaactcactctatgttctaatcactctgatctctgcccctactcaatatttgatttactcttttttcttgcagtttcagctggcatgcggactggtgagtcagcttcatggtcttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncactgagagctgagctcccgggcctggggtgtctctgtgtctttcaggctggctgttgctccaggcccctcggtgggtgttcaaggaggaagaccctattcacctgaggtgtcacagctggaagaacactgctctgcataaggtcacatatttacagaatggcaaagacaggaagtattttcatcataattctgacttccacattccaaaagccacactcaaagatagcggctcctacttctgcagggggcttgttgggagtaaaaatgtgtcttcagagactgtgaacatcaccatcactcaaggtgagacatgtgccaccctggaatgcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntttttcatctctccacttctcctaataggtttggcagtgtcaaccatctcatcattctctccacctgggtaccaagtctctttctgcttggtgatggtactcctttttgcagtggacacaggactatatttctctgtgaagacaaacatttgaagctcaacaagagactggaaggaccataaacttaaatggagaaaggaccctcaagacaaatgacccccatcccatgggagtaataagagcagtggcagcagcatctctgaacatttctctggatttgcaaccccatcatcctcaggcctctctacaagcagcaggaaacatagaactcagagccagatcctttatccaactctcgatttttccttggtctccagtggaagggaaaagcccatgatcttcaagcagggaagccccagtgagtagctgcattcctagaaattgaagtttcagagctacacaaacactttttctgtcccaaccattccctcacagtaaaacaacaatacaggctagggatggtaatcctttaaacatacaaaaattgctcgtattataaattacccagtttagaccggaaaaaagaaaataattattcctaaacaaatggataagtagaattaatgattgaggcaggaccctacagagtgtgggaactgctggggatctagagaattcagtgggaccaatgaaagcatggctgagaaatagcagggtagtccaggagagtctaagggaggtgttcccatctgagcccagagataagggtgtcttcctagaacattagccgtagtggaattaacaggaaatcatgagggtgacgtagaattgagtcttccaggggactctatcagaactggaccatttccaagtatataacgatgagccctctaatgctaggagtagcaaatggtcctaggaaggggactgaggattggggtgggggtggggtggaaaagaaagtacagaacaaaccctgtgtcactgtcccaagttaagctaagtgaacagaactatctcagcatcagaatgagaaagcctgagaagaaagaaccaaccacaagcacacaggaaggaaagcgcaggaggtgaaaatgctttcttggccagggtagtaagaattagaggttaatgcagggactgtaaaaccaccttttctgcttcaatgtctagttcctgtatagctttgttcattgcatttattaaacaaatgttgtataaccaatactaaatgtactactgagcttcactgagttacgctgtgaaactttcaaatccttcttcatgtcagttccaatgaggtggggatggagaagacaattgttgcttatgaaaaaaagctttagctgtctctgttttgtaagctttcagtgcaacatttcttggttccaataaagcattttaca

SEQ ID NO: 9 - protein, 370 aa, Homo sapiens
  MISC_FEATURE: 2B4
MLGQVVTLILLLLLKVYQGKGCQGSADHVVSISGVPLQLQPNSIQTKVDSIAWKKLLPSQNGFHHILKWENGSLPSNTSNDRFSFIVKNLSLLIKAAQQQDSGLYCLEVTSISGKVQTATFQVFVFESLLPDKVEKPRLQGQGKILDRGRCQVALSCLVSRDGNVSYAWYRGSKLIQTAGNLTYLDEEVDINGTHTYTCNVSNPVSWESHTLNLTQDCQNAHQEFRFWPFLVIIVILSALFLGTLACFCVWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 10 - DNA, 279 bp, Homo sapiens
  misc_feature: DAP10
atgatccatctgggtcacatcctcttcctgcttttgctcccagtggctgcagctcagacgactccaggagagagatcatcactccctgccttttaccctggcacttcaggctcttgttccggatgtgggtccctctctctgccgctcctggcaggcctcgtggctgctgatgcggtggcatcgctgctcatcgtgggggcggtgttcctgtgcgcacgcccacgccgcagccccgcccaagatggcaaagtctacatcaacatgccaggcaggggctga

SEQ ID NO: 11 - DNA, 575 bp, Homo sapiens
  misc_feature: DAP12
agacttcctccttcacttgcctggacgctgcgccacatcccaccggcccttacactgtggtgtccagcagcatccggcttcatggggggacttgaaccctgcagcaggctcctgctcctgcctctcctgctggctgtaagtgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaatgagcccgaatcatgacagtcagcaacatgatacctggatccagccattcctgaagcccaccctgcacctcattccaactcctaccgcgatacagacccacagagtgccatccctgagagaccagaccgctccccaatactctcctaaaataaacatgaagcacaaaaacaaaaaaaaaaaaaaaaaa

SEQ ID NO: 12 - DNA, 126 bp, Homo sapiens
  misc_feature: 4-1BB
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 13 - DNA, 339 bp, Homo sapiens
  misc_feature: CD3-zeta
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 14 - protein, 6 aa, Homo sapiens
  MISC_FEATURE: Canonical hemi-tam
  MISC_FEATURE (4)..(5): X = any amino acid
DGYXXL

SEQ ID NO: 15 - protein, 6 aa, Homo sapiens
  MISC_FEATURE: ITSM Motif
  MISC_FEATURE (1)..(1): N = S or T
  MISC_FEATURE (2)..(2): x = any amino acid
  MISC_FEATURE (4)..(5): x = any amino acid
  MISC_FEATURE (6)..(6): N = L or I
NXYXXN

SEQ ID NO: 16 - DNA, 614 bp, Homo sapiens
  misc_feature: Membrane-bound IL15
atggccttaccagtgaccgccttgctcctgccgctggccttgctgctccacgccgccaggccgaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggtatcaccctttactgctaa

SEQ ID NO: 17 - protein, 204 aa, Homo sapiens
  MISC_FEATURE: Membrane-bound IL15
MALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 18 - DNA, 1140 bp, Homo sapiens
  misc_feature: NKG2D/CD8a/4-1BB/CD3z
atggccttaccagtgaccgccttgctcctgccgctggccttgctgctccacgccgccaggccgttattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacatacatctgcatgcaaaggactgtgaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 19 - protein, 379 aa, Homo sapiens
  MISC_FEATURE: Amino acid sequence of NKG2D/CD8a/4-1BB/CD3z
MALPVTALLLPLALLLHAARPLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 20 - protein, 46 aa, Homo sapiens
  MISC_FEATURE: Amino acid for NCR1 TM/IC
MGLAFLVLVALVWFLVEDWLSRKRTRERASRASTWEGRRRLNTQTL

SEQ ID NO: 21 - protein, 276 aa, Homo sapiens
  MISC_FEATURE: Full length NCR2
MAWRALHPLLLLLLLFPGSQAQSKAQVLQSVAGQTLTVRCQYPPTGSLYEKKGWCKEASALVCIRLVTSSKPRTMAWTSRFTIWDDPDAGFFTVTMTDLREEDSGHYWCRIYRPSDNSVSKSVRFYLVVSPASASTQTSWTPRDLVSSQTQTQSCVPPTAGARQAPESPSTIPVPSQPQNSTLRPGPAAPIALVPVFCGLLVAKSLVLSALLVWWGDIWWKTMMELRSLDTQKATCHLQQVTDLPWTSVSSPVEREILYHTVARTKISDDDDEHTL

SEQ ID NO: 22 - protein, 66 aa, Homo sapiens
  MISC_FEATURE: NCR3 TM/IC domains
AGTVLLLRAGFYAVSFLSVAVGSTVYYQGKCLTWKGPRRQLPAVVPAPLPPPCGSSAHLLPPVPGG

SEQ ID NO: 23 - DNA, 741 bp, Homo sapiens
  misc_feature: NKG2D/CD16
atggccttaccagtgaccgccttgctcctgccgctggccttgctgctccacgccgcccgccccctgttcaaccaggaagtgcagatccccctgaccgagtcctattgtggcccttgccctaagaattggatttgctataaaaacaactgctaccagttctttgacgagtctaagaattggtatgagtcccaggcctcttgtatgagccagaacgcctctctgctgaaggtgtacagcaaggaggaccaggatctgctgaagctggtgaagtcctatcactggatgggcctggtgcacatccccacaaacggctcttggcagtgggaggacggctccatcctgtctcctaatctgctgaccatcatcgagatgcagaagggcgattgcgccctgtacgccagctccttcaagggctatatcgagaactgcagcacacccaatacctacatctgtatgcagcggacagtgaccacaaccccagcacccaggccccctacacctgcaccaaccatcgcaagccagccactgtccctgaggcctgaggcatgtaggccagcagcaggaggagcagtgcacacacggggcctggacttcgcctgcgatgtgagcttttgtctggtcatggtgctgctgttcgccgtggataccggcctgtatttttccgtgaagacaaatatccggtctagcaccagagactggaaggatcacaagttcaaatggaggaaggacccacaggacaag

SEQ ID NO: 24 - protein, 247 aa, Homo sapiens
  MISC_FEATURE: NKG2D/CD16
MALPVTALLLPLALLLHAARPLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDVSFCLVMVLLFAVDTGLYFSVKTNIRSSTRDWKDHKFKWRKDPQDK

SEQ ID NO: 25 - DNA, 870 bp, Homo sapiens
  misc_feature: CD8/NKG2DOpt/CD8a/CD16 TM/IC/4-1BB
atggctctgcccgtcaccgcactgctgctgcctctggctctgctgctgcacgccgcacgaccactgttcaatcaggaagtccagatccccctgacagagtcttactgcggcccatgtcccaagaactggatctgctacaagaacaattgttatcagttctttgacgagagcaagaactggtatgagtcccaggcctcttgcatgagccagaatgcctctctgctgaaggtgtacagcaaggaggaccaggatctgctgaagctggtgaagtcctatcactggatgggcctggtgcacatccctacaaacggctcttggcagtgggaggacggctccatcctgtctccaaatctgctgaccatcatcgagatgcagaagggcgattgcgccctgtacgccagctccttcaagggctatatcgagaactgctccacacccaatacctacatctgtatgcagaggaccgtgaccacaacccctgcaccacgcccccctacaccagcacctaccatcgcaagccagcctctgtccctgcggccagaggcatgtagaccagcagcaggaggagcagtgcacacaagaggcctggacttcgcctgcgatgtgagcttttgtctggtcatggtgctgctgttcgccgtggataccggcctgtacttttccgtgaagacaaatatcaggtctagcacccgcgactggaaggatcacaagtttaagtggcggaaggaccctcaggataagaagcggggcagaaagaagctgctgtatatcttcaagcagcccttcatgcggcccgtgcagacaacccaggaggaagacggctgctcatgtagatttcctgaagaagaagaagggggctgtgaactgtaa

SEQ ID NO: 26 - protein, 289 aa, Homo sapiens
  MISC_FEATURE: CD8/NKG2DOpt/CD8a/CD16 TM/IC/4-1BB
MALPVTALLLPLALLLHAARPLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDVSFCLVMVLLFAVDTGLYFSVKTNIRSSTRDWKDHKFKWRKDPQDKKRGRKKLLYIFKQPFMRPVQTTQEEDGCSCRFPEEEEGGCEL

SEQ ID NO: 27 - DNA, 741 bp, Homo sapiens
  misc_feature: NKG2D/NCR1
atggccttaccagtgaccgccttgctcctgccgctggccttgctgctccacgccgcccgccctctgttcaaccaggaagtgcagatccctctgaccgaaagctattgcggaccttgccctaagaattggatttgctataaaaacaactgctaccagttctttgacgagtctaagaattggtatgagtctcaggccagctgtatgtcccagaacgcctctctgctgaaggtgtacagcaaggaggaccaggatctgctgaagctggtgaagtcctatcactggatgggcctggtgcacatccccacaaacggctcttggcagtgggaggacggctctatcctgagccctaatctgctgaccatcatcgagatgcagaagggcgattgcgccctgtacgccagctccttcaagggctatatcgagaactgcagcacacccaatacctacatctgtatgcagaggacagtgaccacaaccccagcaccccgcccccctacacctgcaccaaccatcgcaagccagccactgtccctgcggcctgaggcctgcagaccagcagcaggaggagcagtgcacacccggggcctggacttcgcctgtgatatgggcctggcctttctggtgctggtggccctggtgtggtttctggtggaggattggctgtcccggaagagaacaagggagagggcctcccgggcctctacctgggaaggaagaaggagactgaacacccagacactg

SEQ ID NO: 28 - protein, 247 aa, Homo sapiens
  MISC_FEATURE: NKG2D/NCR1
MALPVTALLLPLALLLHAARPLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDMGLAFLVLVALVWFLVEDWLSRKRTRERASRASTWEGRRRLNTQTL

SEQ ID NO: 29 - DNA, 801 bp, Homo sapiens
  misc_feature: NKG2D/NCR3
atggccttaccagtgaccgccttgctcctgccgctggccttgctgctccacgccgccagacccctgttcaaccaggaggtgcagattcccctgacagaaagctattgtggcccttgccctaaaaattggatttgctataaaaacaactgctaccagttctttgacgagtctaagaattggtatgagtctcaggccagctgtatgtcccagaacgcctctctgctgaaggtgtacagcaaggaggaccaggatctgctgaagctggtgaagtcctatcactggatgggcctggtgcacatccctacaaacggctcttggcagtgggaggacggctctatcctgagcccaaatctgctgaccatcatcgagatgcagaagggcgattgcgccctgtacgccagctccttcaagggctatatcgagaactgcagcacacccaatacctacatctgtatgcagcggacagtgaccacaaccccagcacccagaccccctacacctgcaccaaccatcgccagccagccactgtccctgaggcccgaggcatgcaggcctgcagcaggaggcgccgtgcacacaaggggcctggactttgcctgtgatgcaggaaccgtgctgctgctgagagcaggcttctatgccgtgtcctttctgtctgtggccgtgggctccacagtgtactatcagggcaagtgcctgacctggaagggcccacggagacagctgcccgccgtggtgcccgcccctctgccacccccttgtggcagtagcgcccacctgctgccacccgtgcccggagga

SEQ ID NO: 30 - protein, 267 aa, Homo sapiens
  MISC_FEATURE: NKG2D/NCR3
MALPVTALLLPLALLLHAARPLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDAGTVLLLRAGFYAVSFLSVAVGSTVYYQGKCLTWKGPRRQLPAVVPAPLPPPCGSSAHLLPPVPGG

SEQ ID NO: 31 - protein, 6 aa, Homo sapiens
  MISC_FEATURE (6)..(6): N is an integer indicating the number of GGGGS repeated
GGGGSN

SEQ ID NO: 32 - protein, 60 aa, Homo sapiens
  MISC_FEATURE: GS3/CD8a
GGGGSGGGGSGGGGSTTTPAPRPPTPAPTIASQPLSLRPEACRPAAGGAVHTRGLDFACD

SEQ ID NO: 33 - protein, 45 aa, Homo sapiens
  MISC_FEATURE: GS9
GGGGSGGGGSGGGGSGGGGSGGGGSGGGGSGGGGSGGGGSGGGGS

SEQ ID NO: 34 - protein, 15 aa, Homo sapiens
  MISC_FEATURE: GS3
GGGGSGGGGSGGGGS

SEQ ID NO: 35 - protein, 120 aa, Homo sapiens
  MISC_FEATURE: 2B4 ICR
WRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 36 - DNA, 360 bp, Homo sapiens
  misc_feature: 2B4 ICR
tggaggaggaaaaggaaggagaaacagagcgagacctcccctaaggagttcctgaccatctacgaggacgtgaaggacctgaagaccaggaggaaccacgagcaggaacagacctttcctggcggaggcagcaccatctacagcatgatccagagccagagcagcgcccctaccagccaagagcctgcctacaccctgtacagcctgatccagcccagcaggaaaagcggctccaggaagaggaaccacagccccagcttcaacagcaccatctatgaggtgatcggcaagagccagcccaaggcccagaaccctgccaggctgtccaggaaggagctggagaacttcgacgtgtacagc

SEQ ID NO: 37 - protein, 38 aa, Homo sapiens
  MISC_FEATURE: NKp80 ICR
MQDEDGYMTLNVQSKKRSSAQTSQLTFKDYSVTLHWYK

SEQ ID NO: 38 - DNA, 114 bp, Homo sapiens
  misc_feature: NKp80 ICR
atgcaggatgaggacggctatatgaccctgaacgtccagtccaagaagaggtccagcgctcagaccagccagctgaccttcaaggactactccgtgaccctgcactggtacaag

SEQ ID NO: 39 - protein, 30 aa, Homo sapiens
  MISC_FEATURE: B2Ad N-term ECD
MGQPGNGSAFLLAPNRSHAPDHDVTQQRDE

SEQ ID NO: 40 - DNA, 90 bp, Homo sapiens
  misc_feature: B2 AdR N-term ECD
atggggcaacccgggaacggcagcgccttcttgctggcacccaatagaagccatgcgccggaccacgacgtcacgcagcaaagggacgag

SEQ ID NO: 41 - protein, 33 aa, Homo sapiens
  MISC_FEATURE: B2 AdR TM helix
VWVVGMGIVMSLIVLAIVFGNVLVITAIAKFER

SEQ ID NO: 42 - DNA, 99 bp, Homo sapiens
  misc_feature: B2AdR TM helix
gtgtgggtggtgggcatgggcatcgtcatgtctctcatcgtcctggccatcgtgtttggcaatgtgctggtcatcacagccattgccaagttcgagcgt

SEQ ID NO: 43 - DNA, 924 bp, Homo sapiens
  misc_feature: NK15_1
gccgccaccatggctctgcccgtcaccgcactgctgctgcctctggctctgctgctgcacgccgcacgaccactgttcaatcaggaagtccagatccccctgacagagtcttactgcggcccatgtcccaagaactggatctgctacaagaacaattgttatcagttctttgacgagagcaagaactggtatgagtcccaggcctcttgcatgagccagaatgcctctctgctgaaggtgtacagcaaggaggaccaggatctgctgaagctggtgaagtcctatcactggatgggcctggtgcacatccctacaaacggctcttggcagtgggaggacggctccatcctgtctccaaatctgctgaccatcatcgagatgcagaagggcgattgcgccctgtacgccagctccttcaagggctatatcgagaactgctccacacccaatacctacatctgtatgcagaggaccgtgggtggcggtggctcgggcggtggtgggtcgggtggcggcggatctaccacaacccctgcaccacgcccccctacaccagcacctaccatcgcaagccagcctctgtccctgcggccagaggcatgtagaccagcagcaggaggagcagtgcacacaagaggcctggacttcgcctgcgatgtgagcttttgtctggtcatggtgctgctgttcgccgtggataccggcctgtacttttccgtgaagacaaatatcaggtctagcacccgcgactggaaggatcacaagtttaagtggcggaaggaccctcaggataagaagcggggcagaaagaagctgctgtatatcttcaagcagcccttcatgcggcccgtgcagacaacccaggaggaagacggctgctcatgtagatttcctgaagaagaagaagggggctgtgaactgtaa

SEQ ID NO: 44 - DNA, 789 bp, Homo sapiens
  misc_feature: NK15_2
gccgccaccatggctctgcccgtcaccgcactgctgctgcctctggctctgctgctgcacgccgcacgaccactgttcaatcaggaagtccagatccccctgacagagtcttactgcggcccatgtcccaagaactggatctgctacaagaacaattgttatcagttctttgacgagagcaagaactggtatgagtcccaggcctcttgcatgagccagaatgcctctctgctgaaggtgtacagcaaggaggaccaggatctgctgaagctggtgaagtcctatcactggatgggcctggtgcacatccctacaaacggctcttggcagtgggaggacggctccatcctgtctccaaatctgctgaccatcatcgagatgcagaagggcgattgcgccctgtacgccagctccttcaagggctatatcgagaactgctccacacccaatacctacatctgtatgcagaggaccgtgggtggcggtggctcgggcggtggtgggtcgggtggcggcggatctgtgagcttttgtctggtcatggtgctgctgttcgccgtggataccggcctgtacttttccgtgaagacaaatatcaggtctagcacccgcgactggaaggatcacaagtttaagtggcggaaggaccctcaggataagaagcggggcagaaagaagctgctgtatatcttcaagcagcccttcatgcggcccgtgcagacaacccaggaggaagacggctgctcatgtagatttcctgaagaagaagaagggggctgtgaactgtaa

SEQ ID NO: 45 - DNA, 744 bp, Homo sapiens
  misc_feature: NK15_3
gccgccaccatggctctgcccgtcaccgcactgctgctgcctctggctctgctgctgcacgccgcacgaccactgttcaatcaggaagtccagatccccctgacagagtcttactgcggcccatgtcccaagaactggatctgctacaagaacaattgttatcagttctttgacgagagcaagaactggtatgagtcccaggcctcttgcatgagccagaatgcctctctgctgaaggtgtacagcaaggaggaccaggatctgctgaagctggtgaagtcctatcactggatgggcctggtgcacatccctacaaacggctcttggcagtgggaggacggctccatcctgtctccaaatctgctgaccatcatcgagatgcagaagggcgattgcgccctgtacgccagctccttcaagggctatatcgagaactgctccacacccaatacctacatctgtatgcagaggaccgtggtgagcttttgtctggtcatggtgctgctgttcgccgtggataccggcctgtacttttccgtgaagacaaatatcaggtctagcacccgcgactggaaggatcacaagtttaagtggcggaaggaccctcaggataagaagcggggcagaaagaagctgctgtatatcttcaagcagcccttcatgcggcccgtgcagacaacccaggaggaagacggctgctcatgtagatttcctgaagaagaagaagggggctgtgaactgtaa

SEQ ID NO: 46 - DNA, 1164 bp, Homo sapiens
  misc_feature: NK15_4
gccgccaccatggccctgcctgtgacagccctgctgctgcctctggctctgctgctgcacgctgccagacccttattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgcatgcaaaggactgtgaccacaacccccgctcccagacctcctacccctgcccctacaatcgccagccagcccctgagcctgagacccgaagcctgtagacctgctgccggaggcgctgtgcacacaagaggcctggacttcgcctgcgatatctatatctgggcccctctggctggaacctgtggcgtgctgctgctgagcctggtgattaccaagaggggcaggaagaagctgctgtacatcttcaagcagcctttcatgaggcccgtgcaaaccacccaggaggaggacggctgcagctgcagattccctgaggaggaggagggcggatgcgagctgtggaggaggaaaaggaaggagaaacagagcgagacctcccctaaggagttcctgaccatctacgaggacgtgaaggacctgaagaccaggaggaaccacgagcaggaacagacctttcctggcggaggcagcaccatctacagcatgatccagagccagagcagcgcccctaccagccaagagcctgcctacaccctgtacagcctgatccagcccagcaggaaaagcggctccaggaagaggaaccacagccccagcttcaacagcaccatctatgaggtgatcggcaagagccagcccaaggcccagaaccctgccaggctgtccaggaaggagctggagaacttcgacgtgtacagctga

SEQ ID NO: 47 - DNA, 1155 bp, Homo sapiens
  misc_feature: NK15_5
gccgccaccatggccctgcctgtgacagccctgctgctgcctctggctctgctgctgcacgctgccagacccttattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgcatgcaaaggactgtgatgggacagcctggaaacggcagcgccttcctgctggcccctaacagaagccacgcccccgatcacgatgtgacccagcagagggacgaggtgtgggtggtgggcatgggcatcgtgatgagcctgatcgtgctggctatcgtgttcggcaacgtgctggtgatcaccgccatcgccaagttcgagaggaagaggggcaggaaaaagctgctctacatcttcaagcagcccttcatgaggcccgtgcagaccacccaggaagaggatggctgctcctgtaggtttcccgaggaggaggagggcggctgtgagctgtggaggagaaaaaggaaggagaagcagagcgagaccagccccaaggagttcctgaccatctacgaggacgtgaaggacctgaagaccaggaggaaccacgagcaggaacagaccttccccggcggaggcagcaccatctacagcatgatccagagccagtccagcgcccccacaagccaggaacccgcctacacactgtatagcctgatccagccctccaggaagagcggcagcaggaagaggaaccacagccccagcttcaacagcaccatttacgaggtgatcggaaagagccagcccaaggctcagaaccccgccaggctgagcaggaaggagctcgaaaacttcgacgtgtacagctga

SEQ ID NO: 48 - DNA, 1349 bp, Homo sapiens
  misc_feature: NK15_6
ggatccgaattcgccgccaccatggccctgcctgtgacagccctgctgctgcctctggctctgctgctgcacgctgccagacccttattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgcatgcaaaggactgtgaccacaacccctgctcccagacctcccacacccgcccctacaatcgcctcccagcctctgagcctgagacccgaagcctgtagacctgccgccggcggagctgtgcatacaagaggcctggacttcgcctgcgacatctacatctgggcccctctggctggcacatgcggagtcctgctgctgagcctggtgatcaccaagaggggcaggaagaagctgctgtacatcttcaagcagcccttcatgaggcctgtgcagaccacacaggaggaggacggctgctcctgcaggttccctgaggaggaggagggaggctgcgagctgtggaggaggaagagaaaggagaagcagtccgagacctcccccaaggagttcctcaccatttacgaggacgtgaaggacctgaagaccaggagaaaccacgagcaggaacaaaccttccccggcggcggcagcaccatctacagcatgatccagagccagtcctccgcccctacaagccaggagcctgcctacaccctgtacagcctgatccagcctagcaggaagagcggctccaggaagaggaaccactcccccagcttcaacagcaccatttatgaggtgatcggcaagtcccagcccaaggcccagaaccctgccagactgtccaggaaggagctggagaacttcgacgtctactccggcggcggcggcagcggcggaggaggctccggaggaggcggcagcatgcaggatgaggacggctatatgaccctgaacgtccagtccaagaagaggtccagcgctcagaccagccagctgaccttcaaggactactccgtgaccctgcactggtacaagtgagcggccgcgtcgac

SEQ ID NO: 49 - DNA, 989 bp, Homo sapiens
  misc_feature: NK15_7
ggatccgaattcgccgccaccatggccctgcctgtgacagccctgctgctgcctctggctctgctgctgcatgccgccagacccttattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgcatgcaaaggactgtgaccaccacccctgctcccagaccccctacacctgcccctacaatcgccagccagcccctgagcctgagacctgaggcctgcagacctgctgctggaggcgctgtgcacacaaggggcctcgacttcgcctgcgacatctacatctgggcccctctggccggcacatgtggagtgctgctgctgtccctggtgatcaccaagaggggcaggaagaagctgctgtacatcttcaagcagcccttcatgaggcccgtgcagaccacccaggaggaggacggctgctcctgcagattccccgaggaggaggagggcggatgtgaactgggcggaggaggcagcggcggcggcggcagcggcggcggcggcagcatgcaggatgaggacggctacatgaccctgaacgtgcagagcaagaagaggagcagcgcccagaccagccagctgaccttcaaggactacagcgtgaccctgcactggtacaagtgagcggccgcgtcgac

SEQ ID NO: 50 - DNA, 1430 bp, Homo sapiens
  misc_feature: NK15_8
ggatccgaattcgccgccaccatggccctgcccgtgacagctctgctgctgcctctggccctgctgctgcatgccgctagacccctgttcaaccaggaggtgcagatccccctgaccgaaagctactgcggcccctgccccaagaactggatctgttacaagaacaactgctatcagttcttcgacgagagcaagaactggtacgagagccaggccagctgtatgagccagaacgccagcctgctgaaagtgtatagcaaggaggaccaggacctgctgaagctggtgaagagctaccactggatgggcctggtgcacatccccaccaacggaagctggcagtgggaggacggcagcatcctgagccccaacctgctgaccatcatcgagatgcagaagggcgactgcgccctgtatgccagcagcttcaagggctacatcgagaactgtagcacccccaacacctacatctgcatgcagaggaccgtgggcggcggcggcagcggcggaggcggctccggcggcggcggcagcttattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgcatgcaaaggactgtgatgggccagcctggcaacggcagcgcctttctgctggcccccaacaggagccatgcccctgaccacgacgtgacccagcagagggacgaggtgtgggtggtgggcatgggcatcgtgatgagcctgatcgtgctggccatcgtgttcggcaacgtgctggtgatcaccgccatcgccaagttcgagaggaagaggggcaggaagaagctgctgtacatcttcaagcagcccttcatgagacccgtgcaaaccacccaggaggaggacggctgcagctgcaggtttcccgaggaggaggagggcggatgcgaactgggaggcggaggaagcggaggaggaggatccggaggaggcggaagcatgcaggacgaggacggctacatgaccctgaacgtccagagcaagaagaggagcagcgcccagacctcccagctgaccttcaaggactactccgtgaccctgcactggtacaagtgagcggccgcgtcgac

SEQ ID NO: 51 - DNA, 1439 bp, Homo sapiens
  misc_feature: NK15_9
ggatccgaattcgccgccaccatggccctgcccgtgacagctctgctgctgcctctggccctgctgctgcatgccgctagacccctgttcaaccaggaggtgcagatccccctgaccgaaagctactgcggcccctgccccaagaactggatctgttacaagaacaactgctatcagttcttcgacgagagcaagaactggtacgagagccaggccagctgtatgagccagaacgccagcctgctgaaagtgtatagcaaggaggaccaggacctgctgaagctggtgaagagctaccactggatgggcctggtgcacatccccaccaacggaagctggcagtgggaggacggcagcatcctgagccccaacctgctgaccatcatcgagatgcagaagggcgactgcgccctgtatgccagcagcttcaagggctacatcgagaactgtagcacccccaacacctacatctgcatgcagaggaccgtgggcggcggcggcagcggcggaggcggctccggcggcggcggcagcttattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgcatgcaaaggactgtgaccaccacccctgctcccagaccccctacacctgcccctacaatcgccagccagcccctgagcctgagacctgaggcctgcagacctgctgctggaggcgctgtgcacacaaggggcctcgacttcgcctgcgacatctacatctgggcccctctggccggcacatgtggagtgctgctgctgtccctggtgatcaccaagaggggcaggaagaagctgctgtacatcttcaagcagcccttcatgaggcccgtgcagaccacccaggaggaggacggctgctcctgcagattccccgaggaggaggagggcggatgtgaactgggcggaggaggcagcggcggcggcggcagcggcggcggcggcagcatgcaggatgaggacggctacatgaccctgaacgtgcagagcaagaagaggagcagcgcccagaccagccagctgaccttcaaggactacagcgtgaccctgcactggtacaagtgagcggccgcgtcgac

SEQ ID NO: 52 - DNA, 1329 bp, Homo sapiens
  misc_feature: NK15_10
gccgccacaatggccctgcctgtgacagccctgctgctgcctctggccctgctgctgcatgctgccaggcctctgttcaaccaggaggtgcagatccctctgaccgagagctactgcggcccctgccccaagaactggatctgctacaagaacaactgctaccagttcttcgacgagagcaagaactggtacgagagccaggccagctgcatgtcccagaacgctagcctgctgaaggtgtatagcaaggaggaccaggacctgctgaagctggtgaagagctaccactggatgggcctggtgcacatccccaccaacggctcctggcagtgggaggacggcagcatcctgagccctaacctgctgaccatcatcgagatgcagaagggagactgcgccctgtacgccagctcctttaagggctacatcgagaactgcagcacccccaacacctacatctgtatgcagaggaccgtgggaggcggcggcagcggcggcggcggcagcggcggcggcggcagcttattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgcatgcaaaggactgtgaccaccacccctgcccctagaccccctacacctgcccctaccatcgccagccagcctctgagcctgagacccgaggcctgtagacctgctgccggaggagccgtgcacacaagaggcctggacttcgcctgcgacgtgagcttctgcctggtgatggtgctgctgttcgccgtggacaccggcctgtacttcagcgtgaagaccaacatcaggagcagcaccagggactggaaggaccacaaattcaagtggaggaaggacccccaggacaagaagaggggcaggaagaagctgctgtacatcttcaagcagcccttcatgaggcctgtgcagaccacccaggaggaggacggctgcagctgcaggttccctgaggaggaagagggcggctgcgagctgtga

SEQ ID NO: 53 - DNA, 1239 bp, Homo sapiens
  misc_feature: NK15_11
gccgccaccatggctctgcccgtcaccgcactgctgctgcctctggctctgctgctgcacgccgcacgaccactgttcaatcaggaagtccagatccccctgacagagtcttactgcggcccatgtcccaagaactggatctgctacaagaacaattgttatcagttctttgacgagagcaagaactggtatgagtcccaggcctcttgcatgagccagaatgcctctctgctgaaggtgtacagcaaggaggaccaggatctgctgaagctggtgaagtcctatcactggatgggcctggtgcacatccctacaaacggctcttggcagtgggaggacggctccatcctgtctccaaatctgctgaccatcatcgagatgcagaagggcgattgcgccctgtacgccagctccttcaagggctatatcgagaactgctccacacccaatacctacatctgtatgcagaggaccgtgaccacaacccctgcaccacgcccccctacaccagcacctaccatcgcaagccagcctctgtccctgcggccagaggcatgtagaccagcagcaggaggagcagtgcacacaagaggcctggacttcgcctgcgatgtgagcttttgtctggtcatggtgctgctgttcgccgtggataccggcctgtacttttccgtgaagacaaatatcaggtctagcacccgcgactggaaggatcacaagtttaagtggcggaaggaccctcaggataagaagcggggcagaaagaagctgctgtatatcttcaagcagcccttcatgcggcccgtgcagacaacccaggaggaagacggctgctcatgtagatttcctgaagaagaagaagggggctgtgaactgtggaggaggaaaaggaaggagaaacagagcgagacctcccctaaggagttcctgaccatctacgaggacgtgaaggacctgaagaccaggaggaaccacgagcaggaacagacctttcctggcggaggcagcaccatctacagcatgatccagagccagagcagcgcccctaccagccaagagcctgcctacaccctgtacagcctgatccagcccagcaggaaaagcggctccaggaagaggaaccacagccccagcttcaacagcaccatctatgaggtgatcggcaagagccagcccaaggcccagaaccctgccaggctgtccaggaaggagctggagaacttcgacgtgtacagctga

SEQ ID NO: 54 - DNA, 1064 bp, Homo sapiens
  misc_feature: NK15_12
ggatccgaattcgccgccaccatggctctgcccgtcaccgcactgctgctgcctctggctctgctgctgcacgccgcacgaccactgttcaatcaggaagtccagatccccctgacagagtcttactgcggcccatgtcccaagaactggatctgctacaagaacaattgttatcagttctttgacgagagcaagaactggtatgagtcccaggcctcttgcatgagccagaatgcctctctgctgaaggtgtacagcaaggaggaccaggatctgctgaagctggtgaagtcctatcactggatgggcctggtgcacatccctacaaacggctcttggcagtgggaggacggctccatcctgtctccaaatctgctgaccatcatcgagatgcagaagggcgattgcgccctgtacgccagctccttcaagggctatatcgagaactgctccacacccaatacctacatctgtatgcagaggaccgtgaccacaacccctgcaccacgcccccctacaccagcacctaccatcgcaagccagcctctgtccctgcggccagaggcatgtagaccagcagcaggaggagcagtgcacacaagaggcctggacttcgcctgcgatgtgagcttttgtctggtcatggtgctgctgttcgccgtggataccggcctgtacttttccgtgaagacaaatatcaggtctagcacccgcgactggaaggatcacaagtttaagtggcggaaggaccctcaggataagaagcggggcagaaagaagctgctgtatatcttcaagcagcccttcatgcggcccgtgcagacaacccaggaggaagacggctgctcatgtagatttcctgaagaagaagaagggggctgtgaactgggcggaggaggcagcggcggcggcggcagcggcggcggcggcagcatgcaggatgaggacggctacatgaccctgaacgtgcagagcaagaagaggagcagcgcccagaccagccagctgaccttcaaggactacagcgtgaccctgcactggtacaagtgagcggccgcgtcgac

SEQ ID NO: 55 - protein, 8 aa, Homo sapiens
  MISC_FEATURE: FLAG tag
DYKDDDDK

SEQ ID NO: 56 - protein, 6 aa, Homo sapiens
  MISC_FEATURE: His tag
HHHHHH

SEQ ID NO: 57 - protein, 10 aa, Homo sapiens
  MISC_FEATURE: Myc tag
EQKLISEEDL

SEQ ID NO: 58 - DNA, 753 bp, Homo sapiens
  misc_feature: DNA Sequence Anti-Her2 scFv
gcattcctcctgatcccagatatccagatgacccagtccccgagctccctgtccgcctctgtgggcgatagggtcaccatcacctgccgtgccagtcaggatgtgaatactgctgtagcctggtatcaacagaaaccaggaaaagctccgaaactactgatttactcggcatccttcctctactctggagtcccttctcgcttctctggttccagatctgggacggatttcactctgaccatcagcagtctgcagccggaagacttcgcaacttattactgtcagcaacattatactactcctcccacgttcggacagggtaccaaggtggagatcaaaggcagtactagcggcggtggctccgggggcggatccggtgggggcggcagcagcgaggttcagctggtggagtctggcggtggcctggtgcagccagggggctcactccgtttgtcctgtgcagcttctggcttcaacattaaagacacctatatacactgggtgcgtcaggccccgggtaagggcctggaatgggttgcaaggatttatcctacgaatggttatactagatatgccgatagcgtcaagggccgtttcactataagcgcagacacatccaaaaacacagcctacctgcagatgaacagcctgcgtgctgaggacactgccgtctattattgttctagatggggaggggacggcttctatgctatggactactggggtcaaggaaccctggtcaccgtctcgagt

SEQ ID NO: 59 - protein, 251 aa, Homo sapiens
  MISC_FEATURE: Amino Acid Sequence Anti-Her2 scFv
AFLLIPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGSTSGGGSGGGSGGGGSSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS

SEQ ID NO: 60 - DNA, 1033 bp, Homo sapiens
  misc_feature: DNA Sequence Anti-Her2 scFv adhesion receptor
ggcttccaccatggccttaccagtgaccgccttgctcctgccgctggccttgctgctccacgccgccaggccggcattcctcctgatcccagatatccagatgacccagtccccgagctccctgtccgcctctgtgggcgatagggtcaccatcacctgccgtgccagtcaggatgtgaatactgctgtagcctggtatcaacagaaaccaggaaaagctccgaaactactgatttactcggcatccttcctctactctggagtcccttctcgcttctctggttccagatctgggacggatttcactctgaccatcagcagtctgcagccggaagacttcgcaacttattactgtcagcaacattatactactcctcccacgttcggacagggtaccaaggtggagatcaaaggcagtactagcggcggtggctccgggggcggatccggtgggggcggcagcagcgaggttcagctggtggagtctggcggtggcctggtgcagccagggggctcactccgtttgtcctgtgcagcttctggcttcaacattaaagacacctatatacactgggtgcgtcaggccccgggtaagggcctggaatgggttgcaaggatttatcctacgaatggttatactagatatgccgatagcgtcaagggccgtttcactataagcgcagacacatccaaaaacacagcctacctgcagatgaacagcctgcgtgctgaggacactgccgtctattattgttctagatggggaggggacggcttctatgctatggactactggggtcaaggaaccctggtcaccgtctcgagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgc

SEQ ID NO: 61 - protein, 340 aa, Homo sapiens
  MISC_FEATURE: Amino Acid Anti-Her2 scFv adhesion receptor
ALPVTALLLPLALLLHAARPAFLLIPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGSTSGGGSGGGSGGGGSSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSTTTPAPRPPTPAPTIASQPLSLRPEACRPAAGGAVHTRGLDFACDIYIWAPLAGTCGVLLLSLVITLYC

SEQ ID NO: 62 - DNA, 720 bp, Homo sapiens
  misc_feature: DNA Sequence Anti-PSMA scFv
gaggtgcagctggtgcagagcggcgccgaggtgaagaagcccggcgccagcgtgaagatcagctgcaagaccagcggctacaccttcaccgagtacaccatccactgggtgaagcaggccagcggcaagggcctggagtggatcggcaacatcaaccccaacaacggcggcaccacctacaaccagaagttcgaggacagggccaccctgaccgtggacaagagcaccagcaccgcctacatggagctgagcagcctgaggagcgaggacaccgccgtgtactactgcgccgccggctggaacttcgactactggggccagggcaccaccgtgaccgtgagcagcggcagcaccagcggcggcggcagcggcggcggcagcggcggcggcggcagcagcgacatcgtgatgacccagagccccagcagcctgagcgccagcgtgggcgacagggtgaccatcacctgcaaggccagccaggacgtgggcaccgccgtggactggtaccagcagaagcccggcaaggcccccaagctgctgatctactgggccagcaccaggcacaccggcgtgcccgacaggttcaccggcagcggcagcggcaccgacttcaccctgaccatcagcagcctgcagcccgaggacttcgccgactacttctgccagcagtacaacagctaccccctgaccttcggcggcggcaccaagctggagatcaag

SEQ ID NO: 63 - protein, 240 aa, Homo sapiens
  MISC_FEATURE: Amino Acid Sequence Anti-PSMA scFv
EVQLVQSGAEVKKPGASVKISCKTSGYTFTEYTIHWVKQASGKGLEWIGNINPNNGGTTYNQKFEDRATLTVDKSTSTAYMELSSLRSEDTAVYYCAAGWNFDYWGQGTTVTVSSGSTSGGGSGGGSGGGGSSDIVMTQSPSSLSASVGDRVTITCKASQDVGTAVDWYQQKPGKAPKLLIYWASTRHTGVPDRFTGSGSGTDFTLTISSLQPEDFADYFCQQYNSYPLTFGGGTKLEIK

SEQ ID NO: 64 - DNA, 1000 bp, Homo sapiens
  misc_feature: DNA Sequence Anti- PSMA scFv adhesion receptor
ggcttccaccatggccttaccagtgaccgccttgctcctgccgctggccttgctgctccacgccgccaggccggaggtgcagctggtgcagagcggcgccgaggtgaagaagcccggcgccagcgtgaagatcagctgcaagaccagcggctacaccttcaccgagtacaccatccactgggtgaagcaggccagcggcaagggcctggagtggatcggcaacatcaaccccaacaacggcggcaccacctacaaccagaagttcgaggacagggccaccctgaccgtggacaagagcaccagcaccgcctacatggagctgagcagcctgaggagcgaggacaccgccgtgtactactgcgccgccggctggaacttcgactactggggccagggcaccaccgtgaccgtgagcagcggcagcaccagcggcggcggcagcggcggcggcagcggcggcggcggcagcagcgacatcgtgatgacccagagccccagcagcctgagcgccagcgtgggcgacagggtgaccatcacctgcaaggccagccaggacgtgggcaccgccgtggactggtaccagcagaagcccggcaaggcccccaagctgctgatctactgggccagcaccaggcacaccggcgtgcccgacaggttcaccggcagcggcagcggcaccgacttcaccctgaccatcagcagcctgcagcccgaggacttcgccgactacttctgccagcagtacaacagctaccccctgaccttcggcggcggcaccaagctggagatcaagaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgc

SEQ ID NO: 65 - protein, 329 aa, Homo sapiens
  MISC_FEATURE: Amino Acid Sequence Anti-Her2 scFv adhesion receptor
ALPVTALLLPLALLLHAARPEVQLVQSGAEVKKPGASVKISCKTSGYTFTEYTIHWVKQASGKGLEWIGNINPNNGGTTYNQKFEDRATLTVDKSTSTAYMELSSLRSEDTAVYYCAAGWNFDYWGQGTTVTVSSGSTSGGGSGGGSGGGGSSDIVMTQSPSSLSASVGDRVTITCKASQDVGTAVDWYQQKPGKAPKLLIYWASTRHTGVPDRFTGSGSGTDFTLTISSLQPEDFADYFCQQYNSYPLTFGGGTKLEIKTTTPAPRPPTPAPTIASQPLSLRPEACRPAAGGAVHTRGLDFACDIYIWAPLAGTCGVLLLSLVITLYC

SEQ ID NO: 66 - DNA, 13 bp, Homo sapiens
  misc_feature: Kozack Sequence
ggcttccaccatg

SEQ ID NO: 67 - DNA, 60 bp, Homo sapiens
  misc_feature: CD8 Signal peptide
gccttaccagtgaccgccttgctcctgccgctggccttgctgctccacgccgccaggccg

SEQ ID NO: 68 - protein, 20 aa, Homo sapiens
  MISC_FEATURE: CD8 Signal protein
ALPVTALLLPLALLLHAARP

SEQ ID NO: 69 - DNA, 207 bp, Homo sapiens
  misc_feature: CD8 Transmembrane Domain
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgc

SEQ ID NO: 70 - protein, 69 aa, Homo sapiens
  MISC_FEATURE: CD Transmembrane Protein
TTTPAPRPPTPAPTIASQPLSLRPEACRPAAGGAVHTRGLDFACDIYIWAPLAGTCGVLLLSLVITLYC

(19) United States
(12) Patent Application Publication
(10) Pub. No.: US 2021/0054409 A1
(43) Pub. Date: Feb. 25, 2021

(54) Adhesion Receptor Constructs and Uses Thereof in Natural Killer Cell Immunotherapy

(71) Applicant: National University of Singapore, Singapore (SG)

(72) Inventors: Yansong Zhu, Singapore (SG); Dario Campana, Singapore (SG)

(21) Appl. No.: 16/967,888

(22) PCT Filed: Feb. 7, 2019
(86) PCT No.: PCT/IB2019/000141

§ 371 Date: Aug. 6, 2020

Related U.S. Application Data
(60) Provisional application No. 62/628,797, filed on Feb. 9, 2018.
(60) Provisional application No. 62/736,965, filed on Sep. 26, 2018.

(51) Int. Cl. C12N 15/86; C07K 16/32; C07K 14/705; C12N 5/0783; C07K 16/30; C07K 14/54; A61K 35/17; A61P 35/00

(52) U.S. Cl. CPC C12N 15/86; C07K 16/32; C07K 14/70517; C12N 5/0646; C07K 16/3069; C07K 14/5443; C07K 2317/53; A61P 35/00; C07K 2317/622; C07K 2319/02; C07K 2319/03; C12N 2740/13043; A61K 35/17

(57) ABSTRACT

The invention relates to a composition comprising engineered Natural Killer (NK) cells that express an adhesion receptor comprising an extracellular receptor domain that binds to a target cell antigen as well as a transmembrane domain to anchor the extracellular receptor domain on the surface of the NK cell. The NK cells expressing such adhesion receptor have enhanced ability to target specific cells, such as cancerous cells or those affected by an infectious disease. Several exemplified embodiments relate to NK cells expressing adhesion receptors comprising scFv that targets Her2 or PSMA cancer antigens, the NK cells exhibiting cytotoxic and/or cytolytic effects when the NK cells bind target cells.

### RELATED APPLICATIONS

This application claims the benefit of U.S. Provisional Ser. No. 62/628,797, filed Feb. 9, 2018 and U.S. Provisional Ser. No. 62/736,965, filed Sep. 26, 2018. The entirety of each of these applications is incorporated by reference herein.

### INCORPORATION BY REFERENCE OF MATERIAL IN ASCII TEXT FILE

This application incorporates by reference the Sequence Listing contained in the following ASCII text file being submitted concurrently herewith:
a) File name: 4459_1148_002 Seq List.txt; created Feb. 6, 2019, 86.1 KB in size.

### BACKGROUND

The emergence and persistence of many diseases is characterized by an insufficient immune response to aberrant cells, including malignant and virally infected cells. Immunotherapy is the use and manipulation of the patient's immune system for treatment of various diseases.

### SUMMARY

Immunotherapy presents a new technological advancement in the treatment of disease, wherein immune cells are engineered to express certain targeting and/or effector molecules that specifically identify and react to diseased or damaged cells. This represents a promising advance due, at least in part, to the potential for specifically targeting diseased or damaged cells, as opposed to more traditional approaches, such as chemotherapy, where all cells are impacted, and the desired outcome is that sufficient healthy cells survive to allow the patient to live. One immunotherapy approach is the recombinant expression of adhesion receptors in immune cells to achieve the targeted recognition and destruction of aberrant cells of interest.

To address this need for specifically targeting and destroying, disabling or otherwise rendering inert diseased or infected cells, there are provided for herein polynucleotides, amino acids, and vectors that encode adhesion receptors that impart enhanced targeting and thus, targeted cytotoxicity to immune cells, such as natural killer cells. Also provided for are methods for producing immune cells expressing the adhesion receptors encoded by such polynucleotides, and methods of using the cells to target and destroy diseased or damaged cells.

In several embodiments, there is provided a polynucleotide encoding an adhesion receptor, the adhesion receptor comprising an extracellular receptor domain and a transmembrane domain, wherein the transmembrane domain anchors the extracellular receptor domain on the surface of an immune cell, such as a NK cell.

In several embodiments, the extracellular receptor domain comprises a peptide that enables the extracellular receptor domain to bind a target cell antigen. In several embodiments, the target antigen is differentially expressed on healthy cells as compared to target cells, thereby imparting a degree of specific targeting to cells that express the adhesion receptor. Those cells having such differential (e.g., elevated) expression are thus recognized preferentially and destroyed by immune cells expressing the adhesion receptor, such as, for example, NK cells, T cells, or combinations thereof. In several embodiments, the target cell antigen is associated with a disease, for example a neoplasm, cancer, or tumor. Solid or suspension cancers can be targeted by immune cells expressing the adhesion receptor. In some embodiments, the target cell antigen is a tumor associated antigen, while in additional embodiments the target cell antigen is a tumor specific antigen. The adhesion receptors disclosed herein can also be used to target other antigens, including, but not limited to cells affected with a viral, bacterial, fungal and/or parasitic infection. In such instances, the target cell antigen a viral, bacterial, fungal or parasite antigen.

Non-limiting examples of target cell antigens include bcr-abl, CD19, GD2, GD3, Her-2, K-RAS, MAGE-1, MAGE-10, MAGE-12, MAGE-2, MAGE-3, MAGE-4, MAGE-6, MAGE-A1, MAGE-A2, MAGE-A3, MAGE-A6, MAGE-B1, MAGE-B2, mesothelin, MUC1, MUC16, MUC2, MUM-1, MUM-2, MUM-3, Myosin, NY-ESO, P53, PRAME, PSA, PSCA, PSMA, RAGE, SSX-2, Survivin, Survivin-2B, TGFaRII, TGFbRII, VEGF-R2, and WT1. In one embodiment, the target cell antigen is Her2. In one embodiment, the target cell antigen is PSMA. In one embodiment, the target cell antigen is CD123. In one embodiment, the target cell antigen is GD-2. In one embodiment, the target cell antigen is GD-3. In one embodiment, the target cell antigen is NY-ESO. In one embodiment, the target cell antigen is CD19. In several embodiments, the adhesion receptor does not target CD123 or CD19.

Depending on the embodiment, a variety of different moieties can be used to bind the target cell antigen. For example, in one embodiment the peptide that binds a target cell antigen comprises a monoclonal antibody. In several embodiments, the monoclonal antibody is derived from a hybridoma. Polyclonal antibodies are also used, depending on the embodiment. Recombinant antibodies (e.g., engineered antibodies) are also used, in several embodiments. For example, in several embodiments an antibody developed can be mutated to facilitate its activity or stability when used to treat mammals, such as humans—in other words, the antibody is humanized. In additional embodiment, fragments of an antibody are used, yet retain (or even enhance) binding to the target cell antigen. For example, in several embodiments, a Fab, a Fab′, a F(ab′)2, an Fv, or a single-chain Fv (scFv) are employed. Minibodies, diabodies, and/or single-domain antibodies are also used to target immune cells to a target cell in some embodiments. In some embodiments, the adhesion receptor may not be engineered (e.g., is native to another cell type and expressed as a whole in an NK cell). In several embodiments, the adhesion receptor is not an scFv. In several embodiments, the adhesion receptor is not a de novo binding domain containing polypeptides (DBDpp) that specifically bind a target of interest. Additional information about DBDpp can be found, for example, in International Patent Application PCT/US2016/025868 and/or PCT/US2016/025880, the entire contents of each of which are incorporated by reference herein.

As such, in several embodiments, the peptide that binds a target cell antigen is a single-chain variable fragment (scFv) and the adhesion receptor comprises an anti-Her2 scFv. In some such embodiments, the scFv is encoded by the nucleic acid sequence of SEQ ID NO: 58. In some embodiments, the scFv comprises the amino acid sequence of SEQ ID NO: 59. In one embodiment, the adhesion receptor is encoded by the nucleic acid sequence of SEQ ID NO: 60. In one embodiment, the adhesion receptor comprises the amino acid sequence of SEQ ID NO: 61. In additional embodiments, the adhesion receptor comprises an anti-PSMA scFv. In some such embodiments, the scFv is encoded by the nucleic acid sequence of SEQ ID NO: 62. In some embodiments, the scFv comprises the amino acid sequence of SEQ ID NO: 63. In one embodiment, the adhesion receptor is encoded by the nucleic acid sequence of SEQ ID NO: 64. In one embodiment, the adhesion receptor comprises the amino acid sequence of SEQ ID NO: 65.

While in some embodiments, specific nucleotide or amino acids sequences are used, additional embodiments provided for herein employ nucleotide or amino acids that are about 75%, about 80%, about 85%, about 90%, about 95%, about 98%, or about 99% homologous to such sequences. In some embodiments, the percent homology may vary (e.g., be lower), however the construct retains at least a portion of the function of an adhesion receptor encoded by or having a sequence specifically disclosed herein.

The expression of the adhesion receptor imparts a variety of advantageous characteristics to the immune cells (e.g., NK cells) expressing the receptor. For example, in several embodiments, NK cells that express the adhesion receptor bind target cells more rapidly as compared to NK cells that do not express the adhesion receptor. In several embodiments, the NK cells that express the adhesion receptor have enhanced homing to tumors or infected sites as compared to NK cells that do not express the adhesion receptor. In several embodiments, NK cells that express the adhesion receptor show enhanced cytotoxic activity against cells presenting target cell antigens as compared to NK cells that do not express the adhesion receptor. In several embodiments, NK cells that express the adhesion receptor encoded the polynucleotide have reduced off target cytotoxic effects as compared to NK cells that do not express the adhesion receptor.

In several embodiments, the extracellular receptor domain of the adhesion receptor optionally also includes a second peptide that binds a different target cell antigen than the first peptide. In several embodiments, the extracellular receptor domain optionally also includes a second peptide that binds the same target cell antigen as the first peptide.

In some embodiments, the polynucleotides provided for herein encode more than one adhesion receptor. For example, in some embodiments, a polynucleotide may encode a first and a second adhesion receptor, which in some such embodiments bind different target cell antigens. However, in some embodiments, a first and a second (or more than two) adhesion receptor are designed to bind the same target cell antigen. Even in such embodiments, the adhesion receptors can be configured to bind different epitopes of the same target cell antigen, which can advantageously increase the efficiency of targeting an immune cell to a target cell. Additional biochemical interactions or characteristics are provided for in some embodiments. For example, in some embodiments, the adhesion receptor is configured to dimerize (either homo- or hetero-dimers are possible), which can enhance target affinity. In some embodiments, the extracellular receptor domain further comprises a signal peptide, in order to provide the desired membrane orientation of the receptor domain. In some embodiments, the extracellular receptor domain further comprises a hinge region, which can provide a reduction and/or elimination of steric hindrance that could reduce the effective targeting efficiency of the extracellular receptor domain.

In some embodiments, polynucleotides provided for herein also encode a chimeric receptor. For example, in several embodiments, there is provided a polynucleotide encoding a chimeric receptor comprising an extracellular receptor domain and an effector domain comprising a transmembrane region and an intracellular signaling domain. In some embodiments, the extracellular receptor domain of the chimeric receptor comprises a peptide that binds native ligands of Natural Killer Group 2 member D (NKG2D). In some embodiments, there are also provided polynucleotides that encode membrane-bound interleukin 15 (mbIL15). In some embodiments, a single polynucleotide encodes the adhesion receptor, a chimeric receptor and optionally mbIL15. In additional embodiments, one or more constructs are employed to encode these various elements. In some embodiments, the polynucleotide is an mRNA. In several embodiments, the polynucleotide is operably linked to at least one regulatory element for the expression of the adhesion receptor.

In addition to polynucleotides, there are provided herein vectors that comprise the polynucleotides, the vectors configured to deliver and facilitate the expression of the protein encoded by the polynucleotide in a cell, such as an immune cell (e.g., a NK cell). In several embodiments, the vector is a retrovirus, such as a lentivirus or HIV. Additional embodiments provide for other vectors, such as adenovirus, adeno-associated virus and even non-viral vectors (e.g., liposomes).

Additionally provided for herein are genetically engineered cells, such as immune cells, that comprise the polynucleotide(s) disclosed herein and express the adhesion receptor(s). Various immune cells are employed depending on the embodiment. In several embodiments, NK cells are used. In some embodiments, autologous cells (e.g., NK cells) engineered to express the adhesion receptors are provided. Additional embodiments provide for allogeneic cells (e.g., NK cells) engineered to express the adhesion receptors disclosed herein.

Additionally provided for herein are methods for enhancing NK cell cytotoxicity in a mammal by engineering NK cells expressing an adhesion receptor encoded by a polypeptide provided for herein. Additional embodiments relate to the further provision of enhancing NK cells cytotoxicity by engineering the NK cells to also express a chimeric receptor comprising a ligand binding domain and a signaling domain and/or expressing mbIL15. Depending on the embodiment, the enhanced NK cell cytotoxicity can be leveraged to treat, reduce or otherwise ameliorate a cancer, an infection, or another ailment.

There is also provided for the use of a polynucleotide encoding an adhesion receptor in the manufacture of a cell-based medicament for enhancing Natural Killer (NK) cell cytotoxicity. As discussed above, in the generation of the medicament, the adhesion receptor comprises an extracellular receptor domain configured to bind a target cell antigen, wherein the target cell antigen is differentially expressed between healthy cells and target cells, and a transmembrane domain, wherein the transmembrane domain anchors the extracellular receptor domain on the surface of an NK cell. In several embodiments, the target cell antigen is selected from PSMA, Her2, CD123, GD-2, GD-3, NY-ESO, and CD19. In some embodiments, the extracellular receptor domain that binds the target cell antigen comprises an antibody, a Fab, or an scFv.

The compositions and related methods summarized above and set forth in further detail below describe certain actions taken by a practitioner; however, it should be understood that they can also include the instruction of those actions by another party. Thus, actions such as “administering a population of NK cells expressing an adhesion receptor” include “instructing the administration of a population of NK cells expressing an adhesion receptor.”

### BRIEF DESCRIPTION OF THE DRAWINGS

FIG. 1 depicts a plasmid map illustrating the point of insertion of membrane bound anti-Her2 scFv (mbaHer2) of certain constructs according to several embodiments into the plasmids, illustrated is a Murine Stem Cell Virus (MSCV) plasmid. Depicted is the insertion of a mbaHer2 construct into the EcoRI and XhoI restriction sites of the vector.

FIGS. 2A-C depict flow cytometry data related to the expression of mbaHer2 on the surface of expanded primary NK cells. The mbaHer2 expression profiles of (FIG. 2A) untransduced NK cells, (FIG. 2B) NK cells transduced with a vector containing GFP only, and (FIG. 2C) NK cells transduced with a vector containing anti-Her2 scFv and GFP are depicted. Expression of mbaHer2 was detected by allophycocyanin (APC) conjugated anti-Fab antibody (Y axes). Viral transduction is indicated by green fluorescence protein (GFP) signal (X axes).

FIGS. 3A-B depict data related to 4-hour cytotoxicity assays ata 2:1 effector:target (E:T) ratio of mock transduced NK cells and mbaHer2-expressing NK cells against cancer cell lines expressing (FIG. 3A) high levels of Her2 (Her2 high/intermediate, SKBR3, SKOV3, LNCap, ZR751 and (FIG. 3B) low levels of Her2 (Her2 dim/negative, DU145, PLC/PRF/5).

FIG. 4 depicts data related to the long-term cytotoxicity of NK cells expressing mbaHer2 or GFP only against SKOV3 cells as measured by an IncuCyte live-imaging system (Essen). SKOV3 cells were plated first and NK cells were added 24 hours later at 1:1 E:T. Mean±SD of triplicate measurements is shown.

FIGS. 5A-C depict images of mCherry-labelled SKOV3 cells after 6 days of culture at a 1:1 effector:target ratio (FIG. 5A) without NK cells, (FIG. 5B) with NK cells expressing GFP only, or (FIG. 5C) NK cells expressing mbaHer2.

FIGS. 6A-B depict data related to the tracing of mock transduced NK cells or transduced NK cells expressing mbaHer2 seeded onto SKOV3 cells, with (FIG. 6A) distance travelled and (FIG. 6B) speed measured.

FIGS. 7A-B depict data related to (FIG. 7A) the aggregation of NK cells expressing mbaHer2 with SKOV3 cells by flow cytometer, and (FIG. 7B) quantification of aggregates that are in the Q1-UR quadrant of (FIG. 7A).

FIGS. 8A-H depict data related to the binding of SKOV3 cells to NK cells transduced with a vector containing GFP only (FIGS. 8A-D), and NK cells transduced with a vector containing anti-Her2 scFv and GFP (FIGS. 8E-H). Data were collected by flowing NK cells through attached SKOV cells and examining with immunofluorescence confocal microscopy.

FIGS. 9A-C depict data related to the aggregate cell number of cells (FIG. 9A) with Hoechst staining, (FIG. 9B) GFP expression, and (FIG. 9C) propidium iodide staining from 6 microscope fields of the data of FIG. 8. Data were collected by flowing NK cells through attached SKOV cells and examining with immunofluorescence confocal microscopy.

FIG. 10 depicts a plasmid map illustrating the point of insertion of membrane bound anti-PSMA ScFv (mbaPSMA) of certain constructs according to several embodiments into the plasmids, illustrated is a Murine Stem Cell Virus (MSCV) plasmid. Depicted is the insertion of a mbaPSMA construct into the EcoRI and XhoI restriction sites of the vector.

FIGS. 11A-C depict expression of mbaPSMA on the surface of expanded primary NK cells by flow cytometry. Shown are the mbaPSMA expression profiles of (FIG. 11A) untransduced NK cells, (FIG. 11B) NK cells transduced with a vector containing GFP only, and (FIG. 11C) NK cells transduced with a vector containing anti-PSMA scFv and GFP. Expression of mbaPSMA was detected by allophycocyanin (APC) conjugated anti-Fab antibody (Y axes). Viral transduction is indicated by green fluorescence protein (GFP) signal (X axes).

FIG. 12 provides non-limiting embodiments of constructs and portions thereof according to several embodiments of the invention.

FIG. 13 depicts data related to a cytotoxicity assay used to evaluate the cytotoxic effects of a non-limiting example of a construct disclosed herein against DU145 cells.

FIG. 14 depicts data related to a cytotoxicity assay used to evaluate the cytotoxic effects of a non-limiting example of a construct disclosed herein against LNCap cells.

FIG. 15 depicts data related to in vivo assessment of cytotoxicity against injected SKOV3 cells using a non-limiting example of a construct disclosed herein.

### DETAILED DESCRIPTION

### General

The emergence and persistence of aberrant cells (including virally infected and malignant cells) underlying many diseases is enabled by an insufficient immune response to said aberrant cells. A goal of immunotherapy is to initiate or augment the response of the patient's immune system, for example, to boost the ability of immune cells, such as Natural Killer (NK) cells to damage, kill, or otherwise inhibit damaged or diseased cells. One immunotherapy approach is the recombinant expression of adhesion receptors in immune cells for targeted recognition of the aberrant cells (thus enabling their subsequent destruction). In general, adhesion receptors as described herein comprise an extracellular receptor domain that recognizes ligands on target cells and an anchoring transmembrane domain.

Some embodiments disclosed herein utilize adhesion receptors having that general structure, or having variations in that general structure. As discussed in more detail below, truncations, mutations, additional linkers/spacer elements, dimers, and the like are used, depending on the embodiment, to generate adhesion receptor constructs that exhibit a desired degree of expression in an immune cell (e.g., an NK cell), induce cytotoxic activity from the NK cell, balanced with a degree of target avidity that avoids adverse effects on non-target cells. The recombinant expression of adhesion receptors as disclosed herein on the surface of immune cells can redirect the targeting of immune cells to aberrant cells of interest as well as augment the immune activation upon engagement.

### NK Cells for Immunotherapy

One immunotherapy approach involves administering to patients T cells engineered to express receptors to elicit a positive immune response. However, a drawback of this approach is that it necessitates the use of autologous cells to prevent the induction of graft-versus-host-disease in the patient. As is provided in several embodiments disclosed herein, compositions comprising engineered NK cells enjoy several advantages, such advantages being enhanced by the targeting methodology and compositions disclosed herein. For example, either autologous or donor-derived allogeneic cells can be employed with an NK cell approach. Additionally, according to several embodiments, the engineered NK cells do not significantly increase cytotoxicity against normal cells. Further, NK cells have a significant cytotoxic effect, once activated. In view of this, it is unexpected that the engineered NK cells as provided for herein are able to further elevate that cytotoxic effect, thus providing an even more effective means of selectively killing diseased target cells. Accordingly, in several embodiments, there is provided a method of treating or preventing cancer or an infectious disease, comprising administering a therapeutically effective amount of NK cells expressing the adhesion receptors described herein. In one embodiment, the NK cells administered are autologous cells. In a further embodiment, the NK cells administered are donor-derived (allogeneic) cells.

In several embodiments, engagement and activation of a recombinant NK cell (e.g., by binding to a ligand on a target cell) expressing an adhesion receptor leads to the direct killing of the stressed and/or aberrant cell (e.g., tumor cells, virally-infected cells, etc.) by cytolysis. Accordingly, in several embodiments, there is provided a method of enhancing NK cell cytotoxicity, comprising administering NK cells engineered to express the adhesion receptors described herein. In one embodiment, the NK cells administered are autologous cells. In a further embodiment, the NK cells are donor-derived (allogenic) cells. In several embodiments, engineered NK cells lead to indirect destruction or inhibition of stressed and/or aberrant cell (e.g., tumor cells, virally-infected cells, etc.).

### Extracellular Receptor Domains

As mentioned above, in several embodiments NK cells recognize and destroy aberrant cells, including tumor cells and virally-infected cells. The first stage of NK cell activation is an initial adhesion between the transformed and/or infected cell and the NK cell, of which various extracellular proteins (e.g., selectins and integrins) have been proposed to ligate the two cells together. Once an interface is formed, the cytotoxic activity of these innate immune cells is regulated by the balance of signaling from inhibitory and activating receptors, respectively, that reside on the cell surface. The former bind self-molecules expressed on the surface of healthy cells while the latter bind ligands expressed on aberrant cells. The increased engagement of activating receptors relative to inhibitory receptors leads to NK cell activation and target cell lysis.

The ability of NK cells to recognize and destroy aberrant cells, including tumor cells and virally-infected cells, make it a potentially useful component of immunotherapy approaches (including chimeric receptor-based immunotherapy approaches). However, complicating the use of NK cells is the insufficient delivery of NK cells to target cells, slow rate of NK cell accumulation at target cells, insufficient killing of target cells by NK cells upon engagement, and/or off-target killing of healthy cells. According to several embodiments disclosed herein, polynucleotides encoding adhesions receptors are provided wherein the extracellular receptor domain expressing such a receptor binds an antigen on target cells. In some embodiments, the adhesion receptor is for purposes of target cell binding only (e.g., it does not perform a signaling function). In some embodiments, NK cells expressing the adhesion receptors disclosed herein engage target cells more rapidly (e.g., more quickly, more efficiently, etc.). In some embodiments, NK cells expressing the adhesion receptors disclosed herein have greater cytotoxicity towards target cells (e.g., diseased or damaged cells). In some embodiments, NK cells expressing the adhesion receptors disclosed herein kill a greater portion of target cells. In some embodiments, NK cells expressing the adhesion receptors disclosed herein kill fewer healthy off-target cells.

In some embodiments, the extracellular receptor domain binds a membrane-bound antigen, for example an antigen at the extracellular surface of a cell (e.g., a target cell). In some embodiments, the antigen is a tumor antigen. In some embodiments, the tumor antigen is a tumor-specific antigen (e.g., an antigen that is unique to tumor cells and does not occur in or on other cells in the body). In some embodiments, the tumor antigen is tumor-associated antigen (e.g., an antigen that is not unique to a tumor cell and is also expressed in or on a normal cell under conditions that fail to induce an immune response to the antigen). In some embodiments, the extracellular receptor domain binds an antigen associated with a disease. Antigens can be associated with a disease such as a viral, bacterial, and/or parasitic infection; inflammatory and/or autoimmune disease; or neoplasm such as a cancer and/or tumor.

In some embodiments, the antigen is differentially expressed between healthy cells and target cells. In some embodiments, the expression of the antigen is the same in healthy and target cells, but killing of healthy cells by NK cells expressing the adhesion receptors disclosed herein is minimal because healthy cells lack an NK cell activating ligand profile.

In some embodiments, the extracellular receptor domain comprises an endogenous receptor for the antigen. In some embodiments, the extracellular receptor domain of the adhesion receptor comprises a monoclonal antibody, a polyclonal antibody, a recombinant antibody, a human antibody, a humanized antibody, or a functional derivative, variant or fragment thereof, including, but not limited to, a Fab, a Fab′, a F(ab′)2, an Fv, a single-chain Fv (scFv), minibody, a diabody, and a single-domain antibody such as a heavy chain variable domain (VH), a light chain variable domain (VL) and a variable domain (VHH) of camelid derived Nanobody. In some embodiments, the extracellular receptor domain comprises at least one of a Fab, a Fab′, a F(ab′)2, an Fv, and a scFv. In some embodiments, however neither an scFv nor a de novo binding domain containing polypeptide (DBDpp) that specifically bind a target of interest on a target cell are employed as the adhesion receptor.

In some embodiments, the extracellular receptor domain is configured to bind an antigen associated with, for example a cancer, an infection, or other disease. For example, in several embodiments the extracellular receptor domain one or more of the following antigens: NY-ESO, CD19, CD123, GD-2, GD-3, dectin-1, Her2, and PSMA. Combinations of these antigens are targeted in several embodiments, either by an immune cell expressing a plurality or combination of extracellular receptor domains, or by a population of immune cells expressing a variety of extracellular domains directed to varied antigens. In some embodiments, the adhesion receptor does not target either CD19 or CD123.

Non-limiting examples of antigens which can be bound by the extracellular receptor domain include, but are not limited to, 1-40-β-amyloid, 4-1BB, 5AC, 5T4, 707-AP, A kinase anchor protein 4 (AKAP-4), activin receptor type-2B (ACVR2B), activin receptor-like kinase 1 (ALK1), adenocarcinoma antigen, adipophilin, adrenoceptor u 3 (ADRB3), AGS-22M6, a folate receptor, α-fetoprotein (AFP), AIM-2, anaplastic lymphoma kinase (ALK), androgen receptor, angiopoietin 2, angiopoietin 3, angiopoietin-binding cell surface receptor 2 (Tie 2), anthrax toxin, AOC3 (VAP-1), B cell maturation antigen (BCMA), B7-H3 (CD276), Bacillus anthracis anthrax, B-cell activating factor (BAFF), B-lymphoma cell, bone marrow stromal cell antigen 2 (BST2), Brother of the Regulator of Imprinted Sites (BORIS), C242 antigen, C5, CA-125, cancer antigen 125 (CA-125 or MUC16), Cancer/testis antigen 1 (NY-ESO-1), Cancer/testis antigen 2 (LAGE-1a), carbonic anhydrase 9 (CA-IX), Carcinoembryonic antigen (CEA), cardiac myosin, CCCTC-Binding Factor (CTCF), CCL11 (eotaxin-1), CCR4, CCR5, CD11, CD123, CD125, CD140a, CD147 (basigin), CD15, CD152, CD154 (CD40L), CD171, CD179a, CD18, CD19, CD2, CD20, CD200, CD22, CD221, CD23 (IgE receptor), CD24, CD25 (a chain of IL-2 receptor), CD27, CD274, CD28, CD3, CD31, CD30, CD300 molecule-like family member f (CD300LF), CD319, (SLAMF7), CD33, CD37, CD38, CD4, CD40, CD40 ligand, CD41, CD44 v7, CD44 v8, CD44 v6, CD5, CD51, CD52, CD56, CD6, CD70, CD72, CD74, CD79A, CD79B, CD80, CD97, CEA-related antigen, CFD, ch4D5, chromosome X open reading frame 61 (CXORF61), claudin 18.2 (CLDN18.2), claudin 6 (CLDN6), Clostridium difficile, clumping factor A, CLCA2, colony stimulating factor 1 receptor (CSF1R), CSF2, CTLA-4, C-type lectin domain family 12 member A (CLEC12A), C-type lectin-like molecule-1 (CLL-1 or CLECL1), C—X—C chemokine receptor type 4, cyclin B1, cytochrome P4501B1 (CYP1B1), cyp-B, cytomegalovirus, cytomegalovirus glycoprotein B, dabigatran, DLL4, DPP4, DRS, E. coli shiga toxin type-1, E. coli shiga toxin type-2, ecto-ADP-ribosyltransferase 4 (ART4), EGF-like module-containing mucin-like hormone receptor-like 2 (EMR2), EGF-like-domain multiple 7 (EGFL7), elongation factor 2 mutated (ELF2M), endotoxin, Ephrin A2, Ephrin B2, ephrin type-A receptor 2, epidermal growth factor receptor (EGFR), epidermal growth factor receptor variant III (EGFRvIII), episialin, epithelial cell adhesion molecule (EpCAM), epithelial glycoprotein 2 (EGP-2), epithelial glycoprotein 40 (EGP-40), ERBB2, ERBB3, ERBB4, ERG (transmembrane protease, serine 2 (TMPRSS2) ETS fusion gene), Escherichia coli, ETS translocation-variant gene 6, located on chromosome 12p (ETV6-AML), F protein of respiratory syncytial virus, FAP, Fc fragment of IgA receptor (FCAR or CD89), Fc receptor-like 5 (FCRLS), fetal acetylcholine receptor, fibrin II β chain, fibroblast activation protein a (FAP), fibronectin extra domain-B, FGF-5, Fms-Like Tyrosine Kinase 3 (FLT3), folate binding protein (FBP), folate hydrolase, folate receptor 1, folate receptor α, folate receptor β, Fos-related antigen 1, Frizzled receptor, Fucosyl GM1, G250, G protein-coupled receptor 20 (GPR20), G protein-coupled receptor class C group 5, member D (GPRC5D), ganglioside G2 (GD2), GD3 ganglioside, glycoprotein 100 (gp100), glypican-3 (GPC3), GMCSF receptor α-chain, GPNMB, GnT-V, growth differentiation factor 8, GUCY2C, heat shock protein 70-2 mutated (mut hsp70-2), hemagglutinin, Hepatitis A virus cellular receptor 1 (HAVCR1), hepatitis B surface antigen, hepatitis B virus, HER1, HER2/neu, HER3, hexasaccharide portion of globoH glycoceramide (GloboH), HGF, HHGFR, high molecular weight-melanoma-associated antigen (HMW-MAA), histone complex, HIV-1, HLA-DR, HNGF, Hsp90, HST-2 (FGF6), human papilloma virus E6 (HPV E6), human papilloma virus E7 (HPV E7), human scatter factor receptor kinase, human Telomerase reverse transcriptase (hTERT), human TNF, ICAM-1 (CD54), iCE, IFN-α, IFN-β, IFN-γ, IgE, IgE Fc region, IGF-1, IGF-1 receptor, IGHE, IL-12, IL-13, IL-17, IL-17A, IL-17F, IL-1α, IL-20, IL-22, IL-23, IL-31, IL-31RA, IL-4, IL-5, IL-6, IL-6 receptor, IL-9,immunoglobulin lambda-like polypeptide 1 (IGLL1), influenza A hemagglutinin, insulin-like growth factor 1 receptor (IGF-I receptor), insulin-like growth factor 2 (ILGF2), integrin α4β7, integrin β2, integrin α2, integrin α4, integrin α5β1, integrin α7β7, integrin αIIbβ3, integrin αvβ3, interferon α/β receptor, interferon β-induced protein, Interleukin 11 receptor α (IL-11Rα), Interleukin-13 receptor subunit α-2 (IL-13Rα2 or CD213A2), intestinal carboxyl esterase, kinase domain region (KDR), KIR2D, KIT (CD117), L1-cell adhesion molecule (L1-CAM), legumain, leukocyte immunoglobulin-like receptor subfamily A member 2 (LILRA2), leukocyte-associated immunoglobulin-like receptor 1 (LAIR1), Lewis-Y antigen, LFA-1 (CD11a), LINGO-1, lipoteichoic acid, LOXL2, L-selectin (CD62L), lymphocyte antigen 6 complex, locus K 9 (LY6K), lymphocyte antigen 75 (LY75), lymphocyte-specific protein tyrosine kinase (LCK), lymphotoxin-α (LT-α) or Tumor necrosis factor-β (TNF-β), macrophage migration inhibitory factor (MIF or MMIF), M-CSF, mammary gland differentiation antigen (NY-BR-1), MCP-1, melanoma cancer testis antigen-1 (MAD-CT-1), melanoma cancer testis antigen-2 (MAD-CT-2), melanoma inhibitor of apoptosis (ML-IAP), melanoma-associated antigen 1 (MAGE-A1), mesothelin, mucin 1, cell surface associated (MUC1), MUC-2, mucin CanAg, myelin-associated glycoprotein, myostatin, N-Acetyl glucosaminyl-transferase V (NA17), NCA-90 (granulocyte antigen), nervegrowth factor (NGF), neural apoptosis-regulated proteinase 1, neural cell adhesion molecule (NCAM), neurite outgrowth inhibitor (e.g., NOGO-A, NOGO-B, NOGO-C), neuropilin-1 (NRP1), N-glycolylneuraminic acid, NKG2D, Notch receptor, o-acetyl-GD2 ganglioside (OAcGD2), olfactory receptor 51E2 (OR51E2), oncofetal antigen (h5T4), oncogene fusion protein consisting of breakpoint cluster region (BCR) and Abelson murine leukemia viral oncogene homolog 1 (Abl) (bcr-abl), Oryctolagus cuniculus, OX-40, oxLDL, p53 mutant, paired box protein Pax-3 (PAX3), paired box protein Pax-5 (PAX5), pannexin 3 (PANX3), phosphate-sodium co-transporter, phosphatidylserine, placenta-specific 1 (PLAC1), platelet-derived growth factor receptor α (PDGF-R α), platelet-derived growth factor receptor β (PDGFR-β), polysialic acid, proacrosin binding protein sp32 (OY-TES1), programmed cell death protein 1 (PD-1), proprotein convertase subtilisin/kexin type 9 (PCSK9), prostase, prostate carcinoma tumor antigen-1 (PCTA-1 or Galectin 8), melanoma antigen recognized by T cells 1 (MelanA or MARTI), P15, P53, PRAME, prostate stem cell antigen (PSCA), prostate-specific membrane antigen (PSMA), prostatic acid phosphatase (PAP), prostatic carcinoma cells, prostein, Protease Serine 21 (Testisin or PRSS21), Proteasome (Prosome, Macropain) Subunit, ü Type, 9 (LMP2), Pseudomonas aeruginosa, rabies virus glycoprotein, RAGE, Ras Homolog Family Member C (RhoC), receptor activator of nuclear factor kappa-B ligand (RANKL), Receptor for Advanced Glycation Endproducts (RAGE-1), receptor tyrosine kinase-like orphan receptor 1 (ROR1), renal ubiquitous 1 (RU1), renal ubiquitous 2 (RU2), respiratory syncytial virus, Rh blood group D antigen, Rhesus factor, sarcoma translocation breakpoints, sclerostin (SOST), selectin P, sialyl Lewis adhesion molecule (sLe), sperm protein 17 (SPA17), sphingosine-1-phosphate, squamous cell carcinoma antigen recognized by T Cells 1, 2, and 3 (SART1, SART2, and SART3), stage-specific embryonic antigen-4 (SSEA-4), Staphylococcus aureus, STEAP1, surviving, syndecan 1 (SDC1)+A314, SOX10, survivin, surviving-2B, synovial sarcoma, X breakpoint 2 (SSX2), T-cell receptor, TCR gamma Alternate Reading Frame Protein (TARP), telomerase, TEM1, tenascin C, TGF-β (e.g., TGF-β1, TGF-β2, TGF-β3), thyroid stimulating hormone receptor (TSHR), tissue factor pathway inhibitor (TFPI), Tn antigen ((Tn Ag) or (GalNAcI-Ser/Thr)), TNF receptor family member B cell maturation (BCMA), TNF-1, TRAIL-R1, TRAIL-R2, TRG, transglutaminase 5 (TGS5), tumor antigen CTAA16.88, tumor endothelial marker 1 (TEM1/CD248), tumor endothelial marker 7-related (TEM7R), tumor protein p53 (p53), tumor specific glycosylation of MUC1, tumor-associated calcium signal transducer 2, tumor-associated glycoprotein 72 (TAG72), tumor-associated glycoprotein 72 (TAG-72)+A327, TWEAK receptor, tyrosinase, tyrosinase-related protein 1 (TYRP1 or glycoprotein 75), tyrosinase-related protein 2 (TYRP2), uroplakin 2 (UPK2), vascular endothelial growth factor (e.g., VEGF-A, VEGF-B, VEGF-C, VEGF-D, PIGF), vascular endothelial growth factor receptor 1 (VEGFR1), vascular endothelial growth factor receptor 2 (VEGFR2), vimentin, v-myc avian myelocytomatosis viral oncogene neuroblastoma derived homolog (MYCN), von Willebrand factor (VWF), Wilms tumor protein (WT1), X Antigen Family, Member 1A (XAGE1), 707-AP, a biotinylated molecule, a-Actinin-4, abl-bcr alb-b3 (b2a2), abl-bcr alb-b4 (b3a2), adipophilin, AFP, AIM-2, Annexin II, ART-4, BAGE, b-Catenin, bcr-abl, bcr-abl p190 (e1a2), bcr-abl p210 (b2a2), bcr-abl p210 (b3a2), BING-4, CAG-3, CAIX, CAMEL, Caspase-8, CD171, CD19, CD20, CD22, CD23, CD24, CD30, CD33, CD38, CD44v7/8, CDCl27, CDK-4, CEA, CLCA2, Cyp-B, DAM-10, DAM-6, DEK-CAN, EGFRvIII, EGP-2, EGP-40, ELF2, Ep-CAM, EphA2, EphA3, erb-B2, erb-B3, erb-B4, ES-ESO-1a, ETV6/AML, FBP, fetal acetylcholine receptor, FGF-5, FN, G250, GAGE-1, GAGE-2, GAGE-3, GAGE-4, GAGE-5, GAGE-6, GAGE-7B, GAGE-8, GD2, GD3, GnT-V, Gp100, gp75, Her-2, HLA-A*0201-R170I, HMW-MAA, HSP70-2 M, HST-2 (FGF6), HST-2/neu, hTERT, iCE, IL-11RI, IL-13RT2,KDR, KIAA0205, K-RAS, Ll-cell adhesion molecule, LAGE-1, LDLR/FUT, Lewis Y, MAGE-1, MAGE-10, MAGE-12, MAGE-2, MAGE-3, MAGE-4, MAGE-6, MAGE-A1, MAGE-A2, MAGE-A3, MAGE-A6, MAGE-B1, MAGE-B2, Malic enzyme, Mammaglobin-A, MART-1/Melan-A, MART-2, MC1R, M-CSF, mesothelin, MUC1, MUC16, MUC2, MUM-1, MUM-2, MUM-3, Myosin, NA88-A, Neo-PAP, NKG2D, NPM/ALK, N-RAS, NY-ESO-1, OA1, OGT, oncofetal antigen (h5T4), OS-9, P polypeptide, P15, P53, PRAME, PSA, PSCA, PSMA, PTPRK, RAGE, ROR1, RU1, RU2, SART-1, SART-2, SART-3, SOX10, SSX-2, Survivin, Survivin-2B, SYT/SSX, TAG-72, TEL/AML1, TGFaRII, TGFbRII, TP1, TRAG-3,TRG, TRP-1, TRP-2, TRP-2/INT2, TRP-2-6b, Tyrosinase, VEGF-R2, and WT1. In some embodiments, the extracellular receptor domain binds an antibody which in turn binds an aforementioned antigen. In some embodiments, the extracellular receptor domain binds an Fc domain of an antibody that binds an aforementioned antigen.

In some embodiments, the extracellular receptor domain comprises an antibody or functional derivative, variant or fragment thereof from one or more of the following: 20-(74)-(74) (milatuzumab; veltuzumab), 20-2b-2b, 3F8, 74-(20)-(20) (milatuzumab; veltuzumab), 8H9, A33, AB-16B5, abagovomab, abciximab, abituzumab, ABP 494 (cetuximab biosimilar), abrilumab, ABT-700, ABT-806, Actimab-A (actinium Ac-225 lintuzumab), actoxumab, adalimumab, ADC-1013, ADCT-301, ADCT-402, adecatumumab, aducanumab, afelimomab, AFM13, afutuzumab, AGEN1884, AGS15E, AGS-16C3F, AGS67E, alacizumab pegol, ALD518, alemtuzumab, alirocumab, altumomab pentetate, amatuximab, AMG 228, AMG 820, anatumomab mafenatox, anetumab ravtansine, anifrolumab, anrukinzumab, APN301, APN311, apolizumab, APX003/SIM-BD0801 (sevacizumab), APX005M, arcitumomab, ARX788, ascrinvacumab, aselizumab, ASG-15ME, atezolizumab, atinumab, ATL101, atlizumab (also referred to as tocilizumab), atorolimumab, Avelumab, B-701, bapineuzumab, basiliximab, bavituximab, BAY1129980, BAY1187982, bectumomab, begelomab, belimumab, benralizumab, bertilimumab, besilesomab, Betalutin (177Lu-tetraxetan-tetulomab), bevacizumab, BEVZ92 (bevacizumab biosimilar), bezlotoxumab, BGB-A317, BHQ880, BI 836880, BI-505, biciromab, bimagrumab, bimekizumab, bivatuzumab mertansine, BIW-8962, blinatumomab, blosozumab, BMS-936559, BMS-986012, BMS-986016, BMS-986148, BMS-986178, BNC101, bococizumab, brentuximab vedotin, BrevaRex, briakinumab, brodalumab, brolucizumab, brontictuzumab, C2-2b-2b, canakinumab, cantuzumab mertansine, cantuzumab ravtansine, caplacizumab, capromab pendetide, carlumab, catumaxomab, CBR96-doxorubicin immunoconjugate, CBT124 (bevacizumab), CC-90002, CDX-014, CDX-1401, cedelizumab, certolizumab pegol, cetuximab, CGEN-15001T, CGEN-15022, CGEN-15029, CGEN-15049, CGEN-15052, CGEN-15092, Ch.14.18, citatuzumab bogatox, cixutumumab, clazakizumab, clenoliximab, clivatuzumab tetraxetan, CM-24, codrituzumab, coltuximab ravtansine, conatumumab, concizumab, cR6261, crenezumab, DA-3111 (trastuzumab biosimilar), dacetuzumab, daclizumab, dalotuzumab, dapirolizumab pegol, daratumumab, Daratumumab Enhanze (daratumumab), Darleukin, dectrekumab, demcizumab, denintuzumab mafodotin, denosumab, Depatuxizumab, Depatuxizumab mafodotin, derlotuximab biotin, detumomab, DI-B4, dinutuximab, diridavumab, DKN-01, DMOT4039A, dorlimomab aritox, drozitumab, DS-1123, DS-8895, duligotumab, dupilumab, durvalumab, dusigitumab, ecromeximab, eculizumab, edobacomab, edrecolomab, efalizumab, efungumab, eldelumab, elgemtumab, elotuzumab, elsilimomab, emactuzumab, emibetuzumab, enavatuzumab, enfortumab vedotin, enlimomab pegol, enoblituzumab, enokizumab, enoticumab, ensituximab, epitumomab cituxetan, epratuzumab, erlizumab, ertumaxomab, etaracizumab, etrolizumab, evinacumab, evolocumab, exbivirumab, fanolesomab, faralimomab, farletuzumab, fasinumab, FBTA05, felvizumab, fezakinumab, FF-21101, FGFR2 Antibody-Drug Conjugate, Fibromun, ficlatuzumab, figitumumab, firivumab, flanvotumab, fletikumab, fontolizumab, foralumab, foravirumab, FPA144, fresolimumab, FS102, fulranumab, futuximab, galiximab, ganitumab, gantenerumab, gavilimomab, gemtuzumab ozogamicin, Gerilimzumab, gevokizumab, girentuximab, glembatumumab vedotin, GNR-006, GNR-011, golimumab, gomiliximab, GSK2849330, GSK2857916, GSK3174998, GSK3359609, guselkumab, Hu14.18K322A MAb, hu3S193, Hu8F4, HuL2G7, HuMab-5B1, ibalizumab, ibritumomab tiuxetan, icrucumab, idarucizumab, IGN002, IGN523, igovomab, IMAB362, IMAB362 (claudiximab), imalumab, IMC-CS4, IMC-D11, imciromab, imgatuzumab, IMGN529, IMMU-102 (yttrium Y-90 epratuzumab tetraxetan), IMMU-114, ImmuTune IMP701 Antagonist Antibody, INCAGN1876, inclacumab, INCSHR1210, indatuximab ravtansine, indusatumab vedotin, infliximab, inolimomab, inotuzumab ozogamicin, intetumumab, Ipafricept, IPH4102, ipilimumab, iratumumab, isatuximab, Istiratumab, itolizumab, ixekizumab, JNJ-56022473, JNJ-61610588, keliximab, KTN3379, L19IL2/L19TNF, Labetuzumab, Labetuzumab Govitecan, LAG525, lambrolizumab, lampalizumab, L-DOS47, lebrikizumab, lemalesomab, lenzilumab, lerdelimumab, Leukotuximab, lexatumumab, libivirumab, lifastuzumab vedotin, ligelizumab, lilotomab satetraxetan, lintuzumab, lirilumab, LKZ145, lodelcizumab, lokivetmab, lorvotuzumab mertansine, lucatumumab, lulizumab pegol, lumiliximab, lumretuzumab, LY3164530, mapatumumab, margetuximab, maslimomab, matuzumab, mavrilimumab, MB311, MCS-110, MEDI0562, MEDI-0639, MEDI0680, MEDI-3617, MEDI-551 (inebilizumab), MEDI-565, MEDI6469, mepolizumab, metelimumab, MGB453, MGD006/S80880, MGD007, MGD009, MGD011, milatuzumab, Milatuzumab-SN-38, minretumomab, mirvetuximab soravtansine, mitumomab, MK-4166, MM-111, MM-151, MM-302, mogamulizumab, MOR202, MOR208, MORAb-066, morolimumab, motavizumab, moxetumomab pasudotox, muromonab-CD3, nacolomab tafenatox, namilumab, naptumomab estafenatox, narnatumab, natalizumab, nebacumab, necitumumab, nemolizumab, nerelimomab, nesvacumab, nimotuzumab, nivolumab, nofetumomab merpentan, NOV-10, obiltoxaximab, obinutuzumab, ocaratuzumab, ocrelizumab, odulimomab, ofatumumab, olaratumab, olokizumab, omalizumab, OMP-131R10, OMP-305B83, onartuzumab, ontuxizumab, opicinumab, oportuzumab monatox, oregovomab, orticumab, otelixizumab, otlertuzumab, OX002/MEN1309, oxelumab, ozanezumab, ozoralizumab, pagibaximab, palivizumab, panitumumab, pankomab, PankoMab-GEX, panobacumab, parsatuzumab, pascolizumab, pasotuxizumab, pateclizumab, patritumab, PAT-SC1, PAT-SM6, pembrolizumab, pemtumomab, perakizumab, pertuzumab, pexelizumab, PF-05082566 (utomilumab), PF-06647263, PF-06671008, PF-06801591, pidilizumab, pinatuzumab vedotin, pintumomab, placulumab, polatuzumab vedotin, ponezumab, priliximab, pritoxaximab, pritumumab, PRO 140, Proxinium, PSMA ADC, quilizumab, racotumomab, radretumab, rafivirumab, ralpancizumab, ramucirumab, ranibizumab, raxibacumab, refanezumab, regavirumab, REGN1400, REGN2810/SAR439684, reslizumab, RFM-203, RG7356, RG7386, RG7802, RG7813, RG7841, RG7876, RG7888, RG7986, rilotumumab, rinucumab, rituximab, RM-1929, R07009789, robatumumab, roledumab, romosozumab, rontalizumab, rovelizumab, ruplizumab, sacituzumab govitecan, samalizumab, SAR408701, SAR566658, sarilumab, SAT 012, satumomab pendetide, SCT200, SCT400, SEA-CD40, secukinumab, seribantumab, setoxaximab, sevirumab, SGN-CD19A, SGN-CD19B, SGN-CD33A, SGN-CD70A, SGN-LIV1A, sibrotuzumab, sifalimumab, siltuximab, simtuzumab, siplizumab, sirukumab, sofituzumab vedotin, solanezumab, solitomab, sonepcizumab, sontuzumab, stamulumab, sulesomab, suvizumab, SYD985, SYM004 (futuximab and modotuximab), Sym015, TAB08, tabalumab, tacatuzumab tetraxetan, tadocizumab, talizumab, tanezumab, Tanibirumab, taplitumomab paptox, tarextumab, TB-403, tefibazumab, Teleukin, telimomab aritox, tenatumomab, teneliximab, teplizumab, teprotumumab, tesidolumab, tetulomab, TG-1303, TGN1412, Thorium-227-Epratuzumab Conjugate, ticilimumab, tigatuzumab, tildrakizumab, Tisotumab vedotin, TNX-650, tocilizumab, toralizumab, tosatoxumab, tositumomab, tovetumab, tralokinumab, trastuzumab, trastuzumab emtansine, TRB S07, TRC105, tregalizumab, tremelimumab, trevogrumab, TRPH 011, TRX518, TSR-042, TTI-200.7, tucotuzumab celmoleukin, tuvirumab, U3-1565, U3-1784, ublituximab, ulocuplumab, urelumab, urtoxazumab, ustekinumab, Vadastuximab Talirine, vandortuzumab vedotin, vantictumab, vanucizumab, vapaliximab, varlilumab, vatelizumab, VB6-845, vedolizumab, veltuzumab, vepalimomab, vesencumab, visilizumab, volociximab, vorsetuzumab mafodotin, votumumab, YYB-101, zalutumumab, zanolimumab, zatuximab, ziralimumab, and zolimomab aritox. In some embodiments, the extracellular receptor domain binds an aforementioned antibody. In still further embodiments, the extracellular receptor domain binds an Fc domain of an aforementioned antibody.

Anchoring transmembrane Domain

In some embodiments, the transmembrane domain comprises a polypeptide. The transmembrane domain anchoring the extracellular receptor domain of the adhesion recpetor can have any suitable polypeptide sequence. In some cases, the transmembrane domain comprises a polypeptide sequence of a membrane spanning portion of an endogenous or wild-type membrane spanning protein. In some embodiments, the transmembrane domain comprises a polypeptide sequence having at least 1 (e.g., at least 2, 3, 4, 5, 6, 7, 8, 9, 10 or greater) of an amino acid substitution, deletion, and insertion compared to a membrane spanning portion of an endogenous or wild-type membrane spanning protein. In some embodiments, the transmembrane domain comprises a non-natural polypeptide sequence, such as the sequence of a polypeptide linker. The polypeptide linker may be flexible or rigid. The polypeptide linker can be structured or unstructured. In several embodiments, the chimeric receptor uses a portion of a beta adrenergic receptor as a transmembrane domain.

### Cytoplasmic Effector Domain

In some embodiments, the adhesion receptor further comprises a cytoplasmic effector domain. In some embodiments, the cytoplasmic effector domain comprises a cytoplasmic domain that induces the expansion of the NK cells upon binding of the adhesion receptor to the antigen. In some embodiments, the cytoplasmic effector domain induces the expansion of the NK cells without triggering cytotoxicity. In some embodiments, the cytoplasmic effector domain is the cytoplasmic domain of a cytokine receptor (e.g., IL-2 or IL-15). In some embodiments, such cytoplasmic effector domains are configured to heterodimerize.

### Anti-Her2 Adhesion Receptors

Her2 is a member of the human epidermal growth factor receptor (HER/EGFR/ERBB) family. Amplification or over-expression of this oncogene has been shown to play an important role in the development and progression of certain aggressive types of breast cancer. In some embodiments, the extracellular receptor domain binds to Her2. In some embodiments, the anti-Her2 extracellular receptor domain comprises anti-Her2 antibodies Trastuzumab, Pertuzumab, and functional derivatives, variants or fragments thereof. In several embodiments, the anti-Her2 extracellular receptor comprises an scFv. In several embodiments, the anti-Her2 scFv is encoded by SEQ ID NO. 58. In several embodiments, the anti-Her2 scFv comprises the amino acid sequence of SEQ ID NO: 59. In several embodiments, the extracellular receptor may have one or more additional mutations from SEQ ID NO. 58, but retains, or in some embodiments, has enhanced, Her2-binding function. In several embodiments, the anti-Her2 extracellular receptor domain is provided as a dimer, trimer, or other concatameric format, such embodiments providing enhanced ligand-binding activity. In several embodiments, the sequence encoding the anti-Her2 extracellular receptor domain is optionally fully or partially codon optimized. Additionally, in several embodiments signal peptides are used. The species or sequence of the signal peptide can vary with the construct. However, in several embodiments, the signal peptide of CD8 alpha, or a portion or derivative thereof, is used. In one embodiment, the signal peptide is from CD8a and has the sequence of SEQ ID NO. 4. In one embodiment, the signal peptide is from CD8 and has the DNA sequence of SEQ ID NO: 67. In one embodiment, the signal peptide is from CD8 and has the protein sequence of SEQ ID NO: 68.

### Anti-PSMA Adhesion Receptors

Prostate-specific membrane antigen (PSMA), also known as folate hydrolase 1 (FOLH1), is an integral, non-shed membrane glycoprotein that is highly expressed in prostate epithelial cells and is a cell-surface marker for prostate cancer. In some embodiments, the extracellular receptor domain binds to PSMA. In some embodiments, the anti-PSMA extracellular receptor domain comprises scFv (single-chain Fvs) antibodies, such as: AS, GO, G1, G2, and G4, mAbs 3/E7, 3/F11, 3/A12, K7, K12, and D20; mAbs E99, J591, J533, and J415; mAb 7E11-05.3; antibody 7E11; and antibodies described in Chang et al., 1999, Cancer Res., 59:3192; Murphy et al., 1998, J. Urol., 160:2396; Grauer et al., 1998, Cancer Res., 58:4787; and Wang et al., 2001, Int. J. Cancer, 92:871, and functional derivatives, variants or fragments thereof. In several embodiments, the anti-PSMA extracellular receptor comprises an scFv. In several embodiments, the anti-PSMA scFv is encoded by SEQ ID NO. 62. In several embodiments, the anti-PSMA scFv comprises the amino acid sequence of SEQ ID NO: 63. In several embodiments, the extracellular receptor may have one or more additional mutations from SEQ ID NO. 62, but retains, or in some embodiments, has enhanced, PSMA-binding function. In several embodiments, the extracellular receptor domain is provided as a dimer, trimer, or other concatameric format, such embodiments providing enhanced ligand-binding activity. In several embodiments, the sequence encoding the anti-PSMA extracellular receptor domain is optionally fully or partially codon optimized. Additionally, in several embodiments signal peptides are used. The species or sequence of the signal peptide can vary with the construct. However, in several embodiments, the signal peptide of CD8 alpha is used. In several embodiments, the signal peptide is a portion or derivative of CD8. In one embodiment, the signal peptide is from CD8a and has the sequence of SEQ ID NO. 4. In several embodiments, the signal peptide may have one or more additional mutations from SEQ ID NO. 4, but still provides the desired membrane orientation of the receptor domain. In one embodiment, the signal peptide is from CD8 and has the DNA sequence of SEQ ID NO: 67. In one embodiment, the signal peptide is from CD8 and has the protein sequence of SEQ ID NO: 68.

### Adhesion Receptor Constructs

In view of the disclosure provided herein, there are a variety of adhesion receptors that can be generated and expressed in NK cells in order to target and destroy particular target cells, such as diseased or cancerous cells. Non-limiting examples of such adhesion receptors are discussed in more detail below.

In several embodiments, there are provided polynucleotides encoding an adhesion receptor comprising an extracellular receptor domain and an anchoring transmembrane domain. In several embodiments, there are provided polynucleotides encoding two or more adhesion receptors. In several embodiments, the two or more adhesion receptors act in a synergistic manner to activate (e.g., expand) NK cells upon binding of a ligand to the adhesion receptor. In some embodiments, the two or more adhesion receptors bind different antigens. In some embodiments, the two or more adhesion receptors bind the same antigen. In some embodiments, the two or more adhesion receptors bind different epitopes of the same antigen.

In addition to a single type of adhesion receptor, multiple “repeats” of one type of adhesion receptor and combinations of different types of adhesion receptors, additional co-activating molecules are provided, in several embodiments. For example, in several embodiments, the NK cells are engineered to express membrane-bound interleukin 15 (mbIL15). In such embodiments, the presence of the mbIL15 on the NK cell function to further enhance the cytotoxic effects of the NK cell by synergistically enhancing the proliferation and longevity of the NK cells. In several embodiments, mbIL15 has the nucleic acid sequence of SEQ ID NO. 16. In several embodiments, mbIL15 can be truncated or modified, such that it is at least 70%, at least 75%, at least 80%, at least 85%, at least 90%, at least 95% homologous with the sequence of SEQ ID NO. 16. In several embodiments, the mbIL15 has the amino acid sequence of SEQ ID NO. 17. In several embodiments, mbIL15 can be truncated or modified, such that it is at least 70%, at least 75%, at least 80%, at least 85%, at least 90%, at least 95% homologous with the sequence of SEQ ID NO. 17. In several embodiments, the mbIL15, while truncated, retains at least about 50%, about 60% about 70%, about 80%, about 90%, or about 95% of the function of mbIL15. In conjunction with the adhesion receptors disclosed herein, such embodiments provide particularly effective NK cell compositions for targeting and destroying particular target cells.

In some embodiments the surface expression and efficacy of the adhesion receptors disclosed herein are enhanced by variations in a spacer region (hinge), which, in several embodiments, is located in the extracellular receptor domain adjacent to the transmembrane domain. In some embodiments, domains that serve certain purposes as disclosed elsewhere herein, can serve additional functions (e.g., even though a particular domain may be described in a section disclosing signaling domains, that domain may also be used for another function in a different portion of a construct). For example, in several embodiments, CD8a is repurposed to serve as a hinge region (encoded, in several embodiments, by the nucleic acid sequence of SEQ ID NO: 5). In yet another embodiment, the hinge region comprises an N-terminal truncated form of CD8a and/or a C-terminal truncated form of CD8a. Depending on the embodiment, these truncations can be at least about 50%, at least about 60%, at least about 70%, at least about 80%, or at least about 90% homologous to the hinge encoded by SEQ ID NO. 5. In several additional embodiments, the hinge comprises spans of Glycine and Serine residues (herein termed “GS linkers”) where GSn represents the sequence (Gly-Gly-Gly-Gly-Ser)n (SEQ ID NO. 42). In one embodiment, the hinge comprises both CD8a and GS3, and is encoded by the amino acid sequence of SEQ ID NO: 32, for example, where n=3. In additional embodiments, the value of n may be equal to 1, 2, 4, 5, 6, 7, 8, 9, 10, 11, 12, 13, 14, 15 or greater depending on the embodiment. In several embodiments, the hinge could also be structured as GSn/CD8a. Alternatively, the GS linker can comprise the entire hinge region. In one such embodiment, the hinge region is encoded by the nucleic acid sequence of SEQ ID NO: 33. In another such embodiment, the hinge region is encoded by the nucleic acid sequence of SEQ ID NO: 34.

In several embodiments, the adhesion receptors are configured to dimerize, as discussed in additional detail herein. Dimerization may comprise homodimers or heterodimers, depending on the embodiment. In several embodiments, dimerization results in a shift of avidity of the adhesion receptor (and hence the NK cells expressing the receptor) to better ligand recognition with a coordinate balance in reduced (or lack) of adverse toxic effects. In still further embodiments, the extracellular receptor domain further comprises a CD8a signal peptide. In several embodiments, the adhesion receptors employ internal dimers, or repeats of one or more component subunits.

Optionally, depending on the embodiment, any of the polynucleotides disclosed herein may also encode truncations and/or variants of one or more of the constituent subunits of an adhesion receptor, yet retain their ability to direct NK cells to target cells and in several embodiments unexpectedly enhance cytotoxicity upon binding. In addition, any of the polynucleotides disclosed herein may also optionally include codon-optimized nucleotide sequences encoding the various constituent subunits of an adhesion receptor. As used herein, the terms “fragment” and “truncated” shall be given their ordinary meaning and shall also include N- and C-terminal deletion variants of proteins.

In several embodiments, there are provided polynucleotides encoding an anti-Her2 adhesion receptor, which comprises an anti-Her2 scFv and a transmembrane region. In one embodiment, this adhesion receptor is encoded by the nucleic acid sequence of SEQ ID NO: 60. In yet another embodiment, the anti-Her2 adhesion receptor comprises the amino acid sequence of SEQ ID NO: 61. In several embodiments, this construct is particularly efficacious when the NK cells concurrently express mbIL15, the mbIL15 provides a further synergistic effect with respect to the activation and cytotoxic nature of the NK cells. In some embodiments, the sequence of the adhesion receptor may vary from SEQ ID NO. 60, but remains, depending on the embodiment, at least 70%, at least 75%, at least 80%, at least 85%, at least 90%, or at least 95% homologous with SEQ ID NO. 60. In several embodiments, while the adhesion receptor may vary from SEQ ID NO. 60, the adhesion receptor retains, or in some embodiments, has enhanced, NK cell targeting, activating and/or cytotoxic function.

In several embodiments, there are provided polynucleotides encoding an anti-PSMA adhesion receptor, which comprises an anti-PSMA scFv and a transmembrane region. In one embodiment, this adhesion receptor is encoded by the nucleic acid sequence of SEQ ID NO: 64. In yet another embodiment, the anti-PSMA adhesion receptor comprises the amino acid sequence of SEQ ID NO: 65. In several embodiments, this construct is particularly efficacious when the NK cells concurrently express mbIL15, the mbIL15 provides a further synergistic effect with respect to the activation and cytotoxic nature of the NK cells. In some embodiments, the sequence of the adhesion receptor may vary from SEQ ID NO. 64, but remains, depending on the embodiment, at least 70%, at least 75%, at least 80%, at least 85%, at least 90%, or at least 95% homologous with SEQ ID NO. 64. In several embodiments, while the adhesion receptor may vary from SEQ ID NO. 64, the adhesion receptor retains, or in some embodiments, has enhanced, NK cell targeting, activating and/or cytotoxic function.

The polynucleotides encoding the adhesion receptors described herein may be inserted into vectors to achieve recombinant protein expression in NK cells. In one embodiment, the polynucleotide is operably linked to at least one regulatory element for the expression of the adhesion receptor. In specific embodiments, transcriptional regulatory elements heterologous, such as, for example an internal ribosome entry site (IRES) or enhancer element, to the peptides disclosed herein are employed to direct the transcription of the adhesion receptor. Depending on the embodiment, the various constituent parts of an adhesion receptor can be delivered to an NK cell in a single vector, or alternatively in multiple vectors. In some embodiments, an adhesion receptor construct is delivered in a single vector, while another factor that enhances efficacy of the adhesion receptor, such as mbIL15, is delivered in a separate vector. In several embodiments, an adhesion receptor and a factor that enhances efficacy of the adhesion receptor (e.g., mbIL15), is delivered in a single vector. Regardless of the number of vectors used, any polynucleotide may optionally include a tag sequence, allowing identification of the presence of NK cells expressing the construct. For example, in several embodiments a FLAG tag (DYKDDDDK, SEQ ID NO. 55) is used. Also available are other tag sequences, such as a polyhistidine tag (His-tag) (HHHHHH, SEQ ID NO. 56), HA-tag or myc-tag (EQKLISEEDL; SEQ ID NO: 57). Alternatively, green fluorescent protein, or other fluorescent moiety, is used. Combinations of tag types can also be used, to individually recognize sub-components of an adhesion receptor.

In several embodiments, the polynucleotide encoding the adhesion receptor is an mRNA that may be introduced into NK cells by electroporation. In another embodiment, the vector is a virus, preferably a retrovirus, which may be introduced into NK cells by transduction. In several embodiments, the vector is a Murine Stem Cell Virus (MSCV). In additional embodiments, other vectors may be used, for example lentivirus, adenovirus, adeno-associated virus, and the like may be used. In several embodiments, non-HIV-derived retroviruses are used. The vector chosen will depend upon a variety of factors, including, without limitation, the strength of the transcriptional regulatory elements and the cell to be used to express a protein. The vector can be a plasmid, phagemid, cosmid, viral vector, phage, artificial chromosome, and the like. In additional embodiments, the vectors can be episomal, non-homologously, or homologously integrating vectors, which can be introduced into the appropriate cells by any suitable means (transformation, transfection, conjugation, protoplast fusion, electroporation, calcium phosphate-precipitation, direct microinjection, etc.) to transform them. Other approaches to induce expression of adhesion receptors in NK cells are used in several embodiments, including for example, the SV40 early promoter region, the promoter contained in the 3′ long terminal repeat of Rous sarcoma virus, the herpes thymidine kinase promoter, the regulatory sequences of the metallothionein gene, an adenovirus (ADV) promoter, a cytomegalovirus (CMV) promoter, the bovine papilloma virus (BPV) promoter, the parovirus B 19p6 promoter, the beta-lactamase promoter, the tac promoter, the nopaline synthetase promoter region or the cauliflower mosaic virus 35S RNA promoter, the promoter of ribulose biphosphate carboxylase, the Gal 4 promoter, the ADC (alcohol dehydrogenase) promoter, the PGK (phosphoglycerol kinase) promoter, the synthetic MND promoter containing the U3 region of a modified MoMuLV LTR with the myeloproliferative sarcoma virus enhancer, and the alkaline phosphatase promoter.

Natural killer cells may be engineered to express the adhesion receptors disclosed herein. Adhesion receptor expression constructs may be introduced into NK cells using any of the techniques known to one of skill in the art. In one embodiment, the adhesion receptors are transiently expressed in the NK cells. In another embodiment, the adhesion receptors are stably expressed in NK cells. In an additional embodiment, the NK cells are autologous cells. In yet another embodiment, the NK cells are donor-derived (allogeneic) cells.

Further provided herein are methods of treating a subject having cancer or an infectious disease comprising administering to the subject a composition comprising NK cells engineered to express an adhesion receptor as disclosed herein, the adhesion receptor designed to target a marker or ligand expressed differentially on the damaged or diseased cells or tissue (e.g., expressed to a different degree as compared to a normal cell or tissue). As used herein, the terms “express”, “expressed” and “expression” be given their ordinary meaning and shall refer to allowing or causing the information in a gene or polynucleotide sequence to become manifest, for example producing a protein by activating the cellular functions involved in transcription and translation of a corresponding gene or DNA sequence. The expression product itself, e.g., the resulting protein, may also be said to be “expressed” by the cell. An expression product may be characterized as intracellular, extracellular or transmembrane. The term “intracellular” shall be given its ordinary meaning and shall refer to inside a cell. The term “extracellular” shall be given its ordinary meaning and shall refer to outside a cell. The term “transmembrane” shall be given its ordinary meaning and shall refer to at least a portion of a polypeptide is embedded in a cell membrane. The term “cytoplasmic” shall be given its ordinary meaning and shall refer to residing within the cell membrane, outside the nucleus. As used herein, the terms “treat,” “treating,” and “treatment” in the context of the administration of a therapy to a subject shall be given their ordinary meaning and shall refer to the beneficial effects that a subject derives from a therapy. In certain embodiments, treatment of a subject with a genetically engineered cell(s) described herein achieves one, two, three, four, or more of the following effects, including, for example: (i) reduction or amelioration the severity of disease or symptom associated therewith; (ii) reduction in the duration of a symptom associated with a disease; (iii) protection against the progression of a disease or symptom associated therewith; (iv) regression of a disease or symptom associated therewith; (v) protection against the development or onset of a symptom associated with a disease; (vi) protection against the recurrence of a symptom associated with a disease; (vii) reduction in the hospitalization of a subject; (viii) reduction in the hospitalization length; (ix) an increase in the survival of a subject with a disease; (x) a reduction in the number of symptoms associated with a disease; (xi) an enhancement, improvement, supplementation, complementation, or augmentation of the prophylactic or therapeutic effect(s) of another therapy. Administration can be by a variety of routes, including, without limitation, intravenous, intraarterial, subcutaneous, intramuscular, intrahepatic, intraperitoneal and/or local delivery to an affected tissue. Doses of NK cells can be readily determined for a given subject based on their body mass, disease type and state, and desired aggressiveness of treatment, but range, depending on the embodiments, from about 10′ cells per kg to about 10¹² cells per kg (e.g., 10⁵-10⁷, 10⁷-10¹⁰, 10¹⁰-10¹² and overlapping ranges therein). In one embodiment, a dose escalation regimen is used. In several embodiments, a range of NK cells is administered, for example between about 1×10⁶ cells/kg to about 1×10⁸ cells/kg. Depending on the embodiment, various types of cancer or infection disease can be treated. Various embodiments provided for herein include treatment or prevention of the following non-limiting examples of cancers including, but not limited to, acute lymphoblastic leukemia (ALL), acute myeloid leukemia (AML), adrenocortical carcinoma, Kaposi sarcoma, lymphoma, gastrointestinal cancer, appendix cancer, central nervous system cancer, basal cell carcinoma, bile duct cancer, bladder cancer, bone cancer, brain tumors (including but not limited to astrocytomas, spinal cord tumors, brain stem glioma, craniopharyngioma, ependymoblastoma, ependymoma, medulloblastoma, medulloepithelioma), breast cancer, bronchial tumors, Burkitt lymphoma, cervical cancer, colon cancer, chronic lymphocytic leukemia (CLL), chronic myelogenous leukemia (CIVIL), chronic myeloproliferative disorders, ductal carcinoma, endometrial cancer, esophageal cancer, gastric cancer, Hodgkin lymphoma, non-Hodgkin lymphoma, hairy cell leukemia, renal cell cancer, leukemia, oral cancer, nasopharyngeal cancer, liver cancer, lung cancer (including but not limited to, non-small cell lung cancer, (NSCLC) and small cell lung cancer), pancreatic cancer, bowel cancer, lymphoma, melanoma, ocular cancer, ovarian cancer, pancreatic cancer, prostate cancer, pituitary cancer, uterine cancer, and vaginal cancer.

Further, various embodiments provided for herein include treatment or prevention of the following non-limiting examples of infectious diseases including, but not limited to, infections of bacterial origin may include, for example, infections with bacteria from one or more of the following genera: Bordetella, Borrelia, Brucella, Campylobacter, Chlamydia and Chlamydophila, Clostridium, Corynebacterium, Enterococcus, Escherichia, Francisella, Haemophilus, Helicobacter, Legionella, Leptospira, Listeria, Mycobacterium, Mycoplasma, Neisseria, Pseudomonas, Rickettsia, Salmonella, Shigella, Staphylococcus, Streptococcus, Treponema, Vibrio, and Yersinia, and mutants or combinations thereof. In several embodiments, methods are provided to treat a variety of fungal infections. Depending on the embodiment infections of fungal origin may include, for example, infections with fungi from one or more of the following genera: Aspergillus, Blastomyces, Candida, Coccidioides, Cryptococcus, and Histoplasma, and mutants or combinations thereof. In several embodiments, methods are provided to treat a variety to treat viral infections, such as those caused by one or more viruses, such as adenovirus, Coxsackievirus, Epstein-Barr virus, hepatitis a virus, hepatitis b virus, hepatitis c virus, herpes simplex virus, type 1, herpes simplex virus, type 2, cytomegalovirus, ebola virus, human herpesvirus, type 8, HIV, influenza virus, measles virus, mumps virus, human papillomavirus, parainfluenza virus, poliovirus, rabies virus, respiratory syncytial virus, rubella virus, and varicella-zoster virus.

In some embodiments, also provided herein are nucleic acid and amino acid sequences that have homology of at least 80%, 85%, 90%, 95%, 96%, 97%, 98%, 99% (and ranges therein) as compared with the respective nucleic acid or amino acid sequences of SEQ ID NOS. 1-65 and that also exhibit one or more of the functions as compared with the respective SEQ ID NOS. 1-65: including but not limited to, (i) enhanced proliferation, (ii) enhanced activation, (iii) enhanced cytotoxic activity against cells presenting ligands to which NK cells harboring receptors encoded by the nucleic acid and amino acid sequences bind, (iv) enhanced homing to tumor or infected sites, (v) reduced off target cytotoxic effects, (vi) enhanced secretion of immunostimulatory cytokines and chemokines (including, but not limited to IFNg, TNFa, IL-22, CCL3, CCL4, and CCL5), (vii) enhanced ability to stimulate further innate and adaptive immune responses, and (viii) combinations thereof.

Additionally, in several embodiments, there are provided amino acid sequences that correspond to any of the nucleic acids disclosed herein, while accounting for degeneracy of the nucleic acid code. Furthermore, those sequences (whether nucleic acid or amino acid) that vary from those expressly disclosed herein, but have functional similarity or equivalency are also contemplated within the scope of the present disclosure. The foregoing includes mutants, truncations, substitutions, or other types of modifications.

In several embodiments, the adhesion receptors described herein are co-expressed with a chimeric receptor targeting cells that express natural ligands of Natural Killer Group 2 member D (NKG2D), leading to synergistically enhanced NK cell activation and cytotoxicity. Thus, in several embodiments, there is provided a polynucleotide encoding a NKGD chimeric receptor comprising an extracellular receptor domain, wherein the extracellular receptor domain comprises a peptide that binds native NKG2D, wherein the peptide that binds native ligands of NKG2D is a fragment of NKG2D, a transmembrane region, and an effector domain. In several embodiments, the fragment of NKG2D is encoded by a polynucleotide comprising a fragment of the sequence of SEQ ID NO: 1. In several embodiments, the fragment of NKG2D comprises the sequence of SEQ ID NO: 2, while in additional embodiments, the fragment encoding NKG2D is codon optimized, and comprises, for example, the sequence of SEQ ID NO: 3. In several embodiments, the effector domain comprises one or more of CD16, NCR1, NCR2, NCR3, 4-1BB, CD28, NKp80, CD3zeta and 2B4. In several embodiments, the NKG2D is not full length human NKG2D, but rather a fragment that retains its ability to bind one or more NKG2D ligands. In several embodiments, these effector domains are coupled to CD8 alpha. As discussed herein, combinations of transmembrane and intracellular domains are used in several embodiments and provide for synergistic interactions between the components of the NKG2D chimeric receptor and yield enhanced cytotoxic effects. In several embodiments, linkers, hinges, or other “spacing” elements are provided for in the NKG2D chimeric receptor constructs. For example, in several embodiments, the effector domain comprises a linker. In several embodiments, the polynucleotides encode a GS linker between the portions of the NKG2D chimeric receptor construct, such as between any of 4-1BB, CD28, CD16, NCR1, NCR3, CD3zeta, DAP10, 2B4 or NKp80. In several embodiments, the NKG2D chimeric receptor effector domain comprises a linker. In several embodiments, the polynucleotides encode a GS linker between the portions of the NKG2D chimeric receptor construct, such as between any of 4-1BB, CD28, CD16, NCR1, NCR3, 2B4 or NKp80. In several embodiments, there is provided for a chimeric receptor comprising a hinge region. In several embodiments, the NKG2D chimeric receptor effector domain comprises one or more hemi-ITAM sequences. Additionally, any of chimeric receptors disclosed herein can also be co-expressed with membrane-bound interleukin 15 (mbIL15). In several embodiments, the chimeric receptor does not employ a CD3zeta signaling domain. In several embodiments, the chimeric receptor does not employ an ITAM or hemi-ITAM motif in the signaling domain. In several embodiments, DAP10 is not included in the chimeric receptor.

In several embodiments, the provided polynucleotide is an mRNA. In some embodiments, the polynucleotide is operably linked to at least one regulatory element for the expression of the adhesion receptor. As used herein, the terms “nucleic acid,” “nucleotide,” and “polynucleotide” shall be given their ordinary meanings and shall include deoxyribonucleotides, deoxyribonucleic acids, ribonucleotides, and ribonucleic acids, and polymeric forms thereof, and includes either single- or double-stranded forms. Nucleic acids include naturally occurring nucleic acids, such as deoxyribonucleic acid (“DNA”) and ribonucleic acid (“RNA”) as well as nucleic acid analogs. Nucleic acid analogs include those which include non-naturally occurring bases, nucleotides that engage in linkages with other nucleotides other than the naturally occurring phosphodiester bond or which include bases attached through linkages other than phosphodiester bonds. Thus, nucleic acid analogs include, for example and without limitation, phosphorothioates, phosphorodithioates, phosphorotriesters, phosphoramidates, boranophosphates, methylphosphonates, chiral-methyl phosphonates, 2-O-methyl ribonucleotides, peptide-nucleic acids (PNAs), locked-nucleic acids (LNAs), and the like. As used herein, the term “operably linked,” for example in the context of a regulatory nucleic acid sequence being “operably linked” to a heterologous nucleic acid sequence, shall be given its ordinary meaning and shall mean that the regulatory nucleic acid sequence is placed into a functional relationship with the heterologous nucleic acid sequence. In the context of an IRES, “operably linked to” refers to a functional linkage between a nucleic acid sequence containing an internal ribosome entry site and a heterologous coding sequence initiation in the middle of an mRNA sequence resulting in translation of the heterologous coding sequence. As used herein, the term “vector” shall be given its ordinary meaning and shall refer to a vehicle by which a DNA or RNA sequence (e.g., a foreign gene) can be introduced into a genetically engineered cell, so as to transform the genetically engineered cell and promote expression (e.g., transcription and/or translation) of the introduced sequence. Vectors include viruses, plasmids, phages, etc. The term “adhesion receptor” as used herein shall be given its ordinary meaning and shall refer to a membrane-bound receptor that recognize and bind ligands on target cells (e.g., virally-infected and transformed cells). The term “chimeric receptor” as used herein shall be given its ordinary meaning and shall refer to a cell-surface receptor comprising at least two polypeptide domains not naturally found together on a single protein. The term “chimeric receptor complex” as used herein refers to a first polypeptide, which may comprise at least two polypeptide domains in a combination that are not naturally found together on a single protein, which first polypeptide is associated with a second polypeptide, for example, an adaptor polypeptide, a signaling molecule, or a stimulatory molecule. Additional terms relating to generation and use of adhesion receptors as disclosed here are readily understood by one of ordinary skill in the art and can also be found in International Publication WO 2014/117121 and U.S. Pat. No. 7,994,298, each of which are incorporated by reference in their entirety herein.

Additionally provided, according to several embodiments, is a vector comprising the polynucleotide encoding any of the polynucleotides provided for herein, wherein the polynucleotides are optionally operatively linked to at least one regulatory element for expression of an adhesion receptor. In several embodiments, the vector is a retrovirus.

Further provided herein are engineered natural killer cells comprising the polynucleotide, vector, or adhesion receptors as disclosed herein. In several embodiments, these NK cells are suitable for use in the treatment of prevention of disease, such as, for example, cancer and/or infectious disease.

According to several embodiments, there is provided herein a method for treating or preventing cancer or an infectious disease in a mammal in need thereof, said method comprising administering to said mammal a therapeutically effective amount of NK cells, wherein said NK cells express an adhesion receptor encoded by a polynucleotide according to the present disclosure. In some embodiments, the NK cells are autologous cells isolated from a patient having a cancer or an infectious disease. In some embodiments, the NK cells are allogenic cells isolated from a donor. There is also provided for herein the use of a polynucleotide according to the present disclosure in the manufacture of a medicament for enhancing NK cell cytotoxicity in a mammal in need thereof. There is also provided the use of an isolated genetically engineered natural killer cell according to the present disclosure for treating or preventing cancer or an infectious disease in a mammal in need thereof.

Additionally, in several embodiments, there is provided the use of a polynucleotide encoding an adhesion receptor in the manufacture of a medicament for enhancing Natural Killer (NK) cell cytotoxicity, the adhesion receptor comprising an extracellular receptor domain configured to bind a target cell antigen, wherein the target cell antigen is differentially expressed between healthy cells and target cells, wherein the target cell antigen is PSMA; and a transmembrane domain, wherein the transmembrane domain anchors the extracellular receptor domain on the surface of an NK cell. In several embodiments, the extracellular receptor domain that binds the target cell antigen comprises an antibody, a Fab, or an scFv.

Additionally, in several embodiments, there is provided the use of a polynucleotide encoding an adhesion receptor in the manufacture of a medicament for enhancing Natural Killer (NK) cell cytotoxicity, the adhesion receptor comprising an extracellular receptor domain configured to bind a target cell antigen, wherein the target cell antigen is differentially expressed between healthy cells and target cells, wherein the target cell antigen is Her2; and a transmembrane domain, wherein the transmembrane domain anchors the extracellular receptor domain on the surface of an NK cell. In several embodiments, the extracellular receptor domain that binds the target cell antigen comprises an antibody, a Fab, or an scFv.

Additionally, in several embodiments, there is provided the use of a polynucleotide encoding an adhesion receptor in the manufacture of a medicament for enhancing Natural Killer (NK) cell cytotoxicity, the adhesion receptor comprising an extracellular receptor domain configured to bind a target cell antigen, wherein the target cell antigen is differentially expressed between healthy cells and target cells, wherein the target cell antigen is CD123; and a transmembrane domain, wherein the transmembrane domain anchors the extracellular receptor domain on the surface of an NK cell. In several embodiments, the extracellular receptor domain that binds the target cell antigen comprises an antibody, a Fab, or an scFv.

Additionally, in several embodiments, there is provided the use of a polynucleotide encoding an adhesion receptor in the manufacture of a medicament for enhancing Natural Killer (NK) cell cytotoxicity, the adhesion receptor comprising an extracellular receptor domain configured to bind a target cell antigen, wherein the target cell antigen is differentially expressed between healthy cells and target cells, wherein the target cell antigen is GD-2; and a transmembrane domain, wherein the transmembrane domain anchors the extracellular receptor domain on the surface of an NK cell. In several embodiments, the extracellular receptor domain that binds the target cell antigen comprises an antibody, a Fab, or an scFv.

Additionally, in several embodiments, there is provided the use of a polynucleotide encoding an adhesion receptor in the manufacture of a medicament for enhancing Natural Killer (NK) cell cytotoxicity, the adhesion receptor comprising an extracellular receptor domain configured to bind a target cell antigen, wherein the target cell antigen is differentially expressed between healthy cells and target cells, wherein the target cell antigen is GD-3; and a transmembrane domain, wherein the transmembrane domain anchors the extracellular receptor domain on the surface of an NK cell. In several embodiments, the extracellular receptor domain that binds the target cell antigen comprises an antibody, a Fab, or an scFv.

Additionally, in several embodiments, there is provided the use of a polynucleotide encoding an adhesion receptor in the manufacture of a medicament for enhancing Natural Killer (NK) cell cytotoxicity, the adhesion receptor comprising an extracellular receptor domain configured to bind a target cell antigen, wherein the target cell antigen is differentially expressed between healthy cells and target cells, wherein the target cell antigen is NY-ESO; and a transmembrane domain, wherein the transmembrane domain anchors the extracellular receptor domain on the surface of an NK cell. In several embodiments, the extracellular receptor domain that binds the target cell antigen comprises an antibody, a Fab, or an scFv.

Additionally, in several embodiments, there is provided the use of a polynucleotide encoding an adhesion receptor in the manufacture of a medicament for enhancing Natural Killer (NK) cell cytotoxicity, the adhesion receptor comprising an extracellular receptor domain configured to bind a target cell antigen, wherein the target cell antigen is differentially expressed between healthy cells and target cells, wherein the target cell antigen is CD19; and a transmembrane domain, wherein the transmembrane domain anchors the extracellular receptor domain on the surface of an NK cell. In several embodiments, the extracellular receptor domain that binds the target cell antigen comprises an antibody, a Fab, or an scFv.

In several embodiments there is provided a polynucleotide encoding an anti-Her2 single chain variable fragment (anti-Her2 scFv). In several embodiments, the anti-Her2 scFv comprises SEQ ID NO: 59. In several embodiments, the anti-Her2 scFv is encoded by a nucleic acid comprising SEQ ID NO: 58. In several embodiments, the polynucleotide further encodes a transmembrane domain. In several embodiments, the transmembrane domain comprises a CD8 transmembrane domain. In several embodiments, the CD8 transmembrane domain comprises SEQ ID NO. 70. In several embodiments, the CD8 transmembrane domain is encoded by a nucleic acid comprising SEQ ID NO: 69. In several embodiments, the polynucleotide further encodes a signal peptide. In several embodiments, the signal peptide comprises the CD8 signal peptide of SEQ ID NO: 68. In several embodiments, the polynucleotide further comprises a Kozak sequence. In several embodiments, the Kozak sequence comprises the nucleic acid sequence of SEQ ID NO: 66.

In several embodiments, there is provided a polynucleotide encoding an anti-PSMA single chain variable fragment (anti-PSMA scFv). In several embodiments, the anti-PSMA scFv comprises SEQ ID NO: 63. In several embodiments, the anti-PSMA scFv is encoded by a nucleic acid comprising SEQ ID NO: 62. In several embodiments, the polynucleotide further encodes a transmembrane domain. In several embodiments, the transmembrane domain comprises a CD8 transmembrane domain. In several embodiments, the CD8 transmembrane domain comprises SEQ ID NO. 70. In several embodiments, the CD8 transmembrane domain is encoded by a nucleic acid comprising SEQ ID NO: 69. In several embodiments, the polynucleotide further encodes a signal peptide. In several embodiments, the signal peptide comprises the CD8 signal peptide of SEQ ID NO: 68. In several embodiments, the polynucleotide further comprises a Kozak sequence. In several embodiments, the Kozak sequence comprises the nucleic acid sequence of SEQ ID NO: 66.

In several embodiments, there is provided a polynucleotide encoding an anti-Her2 single chain variable fragment (anti-Her2 scFv), wherein the anti-Her2 scFv comprises SEQ ID NO: 59. In several embodiments, the anti-Her2 scFv further comprises a transmembrane domain. In several embodiments, the transmembrane domain comprise a CD8 transmembrane domain. In several embodiments, the CD8 transmembrane domain comprises SEQ ID NO. 70. In several embodiments the anti-Her2 scFv further comprises a signal peptide. In several embodiments, the signal peptide comprises the CD8 signal peptide of SEQ ID NO: 68.

In several embodiments, there is provided an anti-PSMA single chain variable fragment (anti-PSMA scFv), wherein the anti-PSMA scFv comprises SEQ ID NO: 63. In several embodiments, the anti-PSMA scFv further comprises a transmembrane domain. In several embodiments, the transmembrane domain comprise a CD8 transmembrane domain. In several embodiments, the CD8 transmembrane domain comprises SEQ ID NO. 70. In several embodiments, the anti-PSMA scFv further comprises a signal peptide. In several embodiments, the signal peptide comprises the CD8 signal peptide of SEQ ID NO: 68.

### EXAMPLES

### Methods

The following experimental methods and materials were used in the non-limiting experimental examples disclosed below.

### Cell Lines and Culture Conditions

The human tumor cell lines SKBR3, SKOV3, LNCap, ZR751, DU145, and PLC/PRF/5 were purchased from the American Type Culture Collection (ATCC; Rockville, Md.). Cell lines were maintained in RPMI-1640 (ThermoFisher, Waltham, Mass.); media were supplemented with 10% fetal bovine serum (FBS; GE Healthcare, Chicago, Ill.) and antibiotics. The cell lines were transduced with a murine stem cell virus (MSCV) retroviral vector (from the Vector Development and Production Shared Resource of St. Jude Children's Research Hospital, Memphis, Tenn.) containing either green fluorescence protein (GFP) and luciferase, or mCherry.

### Expansion of Human NK Cells

Peripheral blood samples were obtained from discarded anonymized by-products of platelet donations from healthy adult donors at the National University Hospital Blood Bank, Singapore.

Mononucleated cells were separated by centrifugation on a Lymphoprep density step (Nycomed, Oslo, Norway) and washed twice in RPMI-1640. To expand NK cells, mononucleated cells were co-cultured with genetically-modified K562-mb15-41BBL cells. Briefly, peripheral blood mononucleated cells (3×10⁶) were cultured in a 6-well tissue culture plate with 2×10⁶ irradiated (100 Gy) K562-mb15-41BBL cells in SCGM medium (CellGenix, Freiburg, Germany) containing 10% FBS and 40 IU/mL human interleukin (IL)-2 (Novartis, Basel, Switzerland). Every 2-3 days, fresh tissue culture medium and IL-2 was added. After 7 days of co-culture, residual T cells were removed using Dynabeads CD3 (Thermo Fisher), producing cell populations containing >90% CD56+ CD3− NK cells. Expanded NK cells were maintained in SCGM with FBS, antibiotics, and 400 IU/mL IL2 before the experiments.

### DNA Plasmids, Production of Retrovirus and Transduction of NK Cells

Plasmids encoding the anti-Her2-CD8tm and anti-PSMA-CD8tm constructs were synthesized by Genescript (Nanjing, China). A RD114-pseudotyped MSCV retrovirus containing the anti-Her2-CD8tm or anti-PSMA-CD8tm constructs was used to transduce NK cells. Retroviral vector-conditioned medium was added to RetroNectin (Takara, Otsu, Japan)-coated polypropylene tubes; after centrifugation and removal of the supernatant, expanded NK cells (5×10⁵) were added to the tubes and left at 37° C. for 12 hours; fresh viral supernatant was added every 12 hours for a total of 6 times. Cells were then maintained in RPMI with FBS, antibiotics and 400 IU/ml of IL2 until the time of the experiments.

### Detection of Adhesion Receptor Expression by Flow Cytometry

Surface expression of the anti-PSMA-CD8tm construct was detected with a biotin-conjugated goat anti-mouse IgG F(ab′)₂ fragment-specific antibody (Jackson ImmunoResearch, West Grove, Pa.). Surface expression of anti-Her2-CD8tm was detected with Protein L-biotin (Genescript). Either reagent was visualized with phycoerythrin (PE)- or APC-conjugated streptavidin (Jackson ImmunoResearch). Cell staining was analysed in an Accuri C6 flow cytometer (Becton Dickinson).

Detection of Aggregation of NK cells with Tumor Cells by Flow Cytometry

To measure cell-to-cell aggregation, SKOV3 cells transduced with mCherry were trypsinized, washed twice and transferred to a 1.5 ml Eppendorf tube. NK cells transduced with either anti-Her2-CD8tm plus GFP, or GFP alone (“mock”), were added at 1:1 ratio. After 30 minutes at 37° C. 5% CO2, the cell suspension was vortexed for 10 seconds and then the proportions of doublets mCherry+GFP+ were counted by flow cytometry.

### Detection of Binding of NK Cells and Tumor Cells

SKOV3 cells were seeded into Ibidi 1 micro-slide (Ibidi, Martinsried, Germany) and grown to confluence. After staining with Hoechst 33342 solution for 10 minutes, cells were washed twice with RPMI. NK cells expressing anti-Her2-CD8tm or GFP alone were added to each channel (1×10⁵). After 10 minutes, non-adherent NK cells were removed from both ends of the chamber, the channel was washed twice with RPMI, propidium iodide was added and cells were examined using an Olympus FluoView FV1000 confocal microscope.

### Cytotoxicity Assays

Target cells transduced with GFP/luciferin were suspended in RPMI-1640 with 10% FBS, and plated into 96-well flat bottom plates (Costar, Corning, N.Y.). The plates were placed in an incubator for at least 4 hours to allow for cell attachment before adding NK cells. Expanded NK cells expressing with anti-Her2-CD8tm or GFP alone, suspended in RPMI-1640 with 10% FBS were then added at various effector-to-target (E:T) ratios, and co-cultured with target cells for 4 hours. At the end of the cultures, number of viable cells were measured, after adding BrightGlo (Promega, Fitchburg, Wis.) to the wells, using a Flx 800 plate reader (BioTek, Winooski, Vt.). In some tests, cytotoxicity was measured using an IncuCyte Zoom System (Essen BioScience). SKOV3 cells transduced with mCherry were cultured alone or with NK cells transduced with either anti-Her2-CD8tm or GFP alone. The number of viable target cells in triplicate cultures for each condition was measured every 4 hours for 160 hours. Cell images were also recorded using the same instrument.

### Example 1—Anti-Her2 Adhesion Receptor Construct

As disclosed herein, various adhesion receptor constructs comprising an extracellular receptor domain coupled with various transmembrane domains are provided. The present experiment was conducted to evaluate the expression and cytotoxic activity of an anti-Her2 adhesion receptor construct. The anti-Her2 adhesion receptor construct was prepared and tested according to the methods and materials described above. Depending on the construct, the methods used can be readily adjusted to account for variations required for generating, expressing and testing a construct.

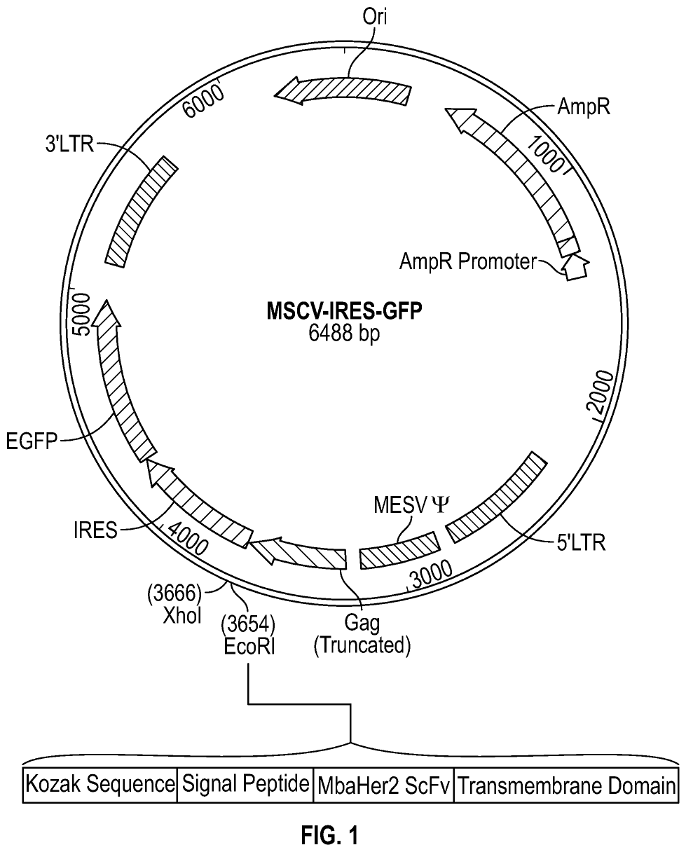

FIG. 1 depicts a plasmid map illustrating the point of insertion of membrane bound anti-Her2 scFv (mbaHer2) into a Murine Stem Cell Virus (MSCV) plasmid. This plasmid map shows the insertion of the mbaHer2 construct into the EcoRI and XhoI restriction sites of the vector. The extracellular receptor domain of the mbaHer2 construct comprises an anti-Her2 single chain variable fragment (anti-Her2 scFv) and CD8 signal peptide that provides the desired membrane orientation of the receptor domain. The CD8 signal peptide comprises the amino acid sequence of SEQ ID NO: 68 and is encoded by the nucleic acid sequence of SEQ ID NO: 67. The anti-Her2 scFv comprises the amino acid sequence of SEQ ID NO: 59 and is encoded by the nucleic acid sequence of SEQ ID NO: 58. As shown in FIG. 1, the mbaHer2 construct further comprises a CD8 transmembrane domain downstream of the extracellular receptor domain that anchors the extracellular receptor domain on the surface of an NK cell. The CD8 transmembrane domain comprises the amino acid sequence of SEQ ID NO. 70 and is encoded by a nucleic acid of SEQ ID NO. 69. As shown in FIG. 1, the mbaHer2 construct further comprises a Kozak sequence of SEQ ID NO. 66 upstream of the signal peptide. Collectively, the mbaHer2 construct encodes an adhesion receptor that comprises the amino acid sequence of SEQ ID: 61 and is encoded by the nucleic acid sequence of SEQ ID NO. 60.

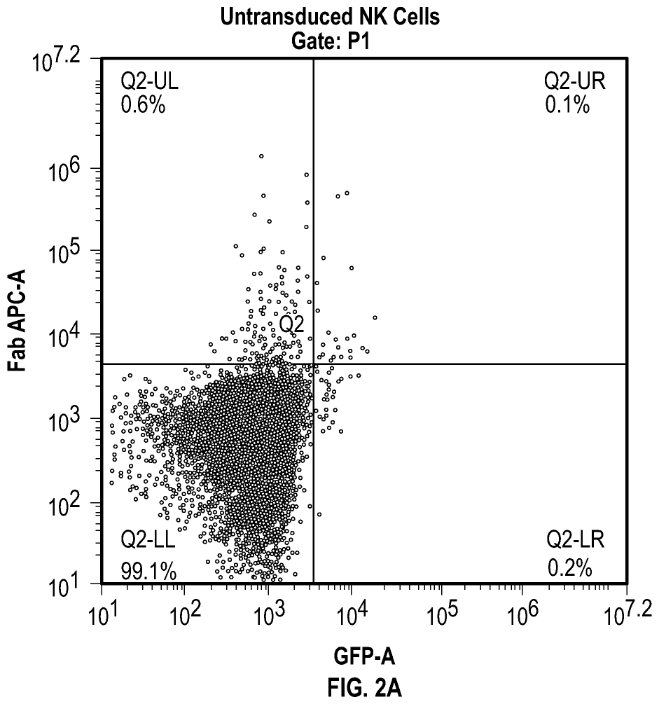

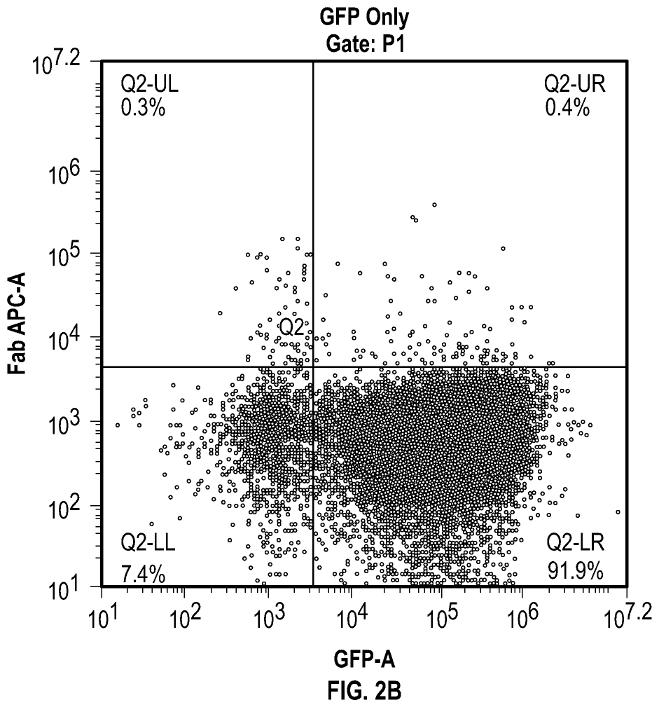

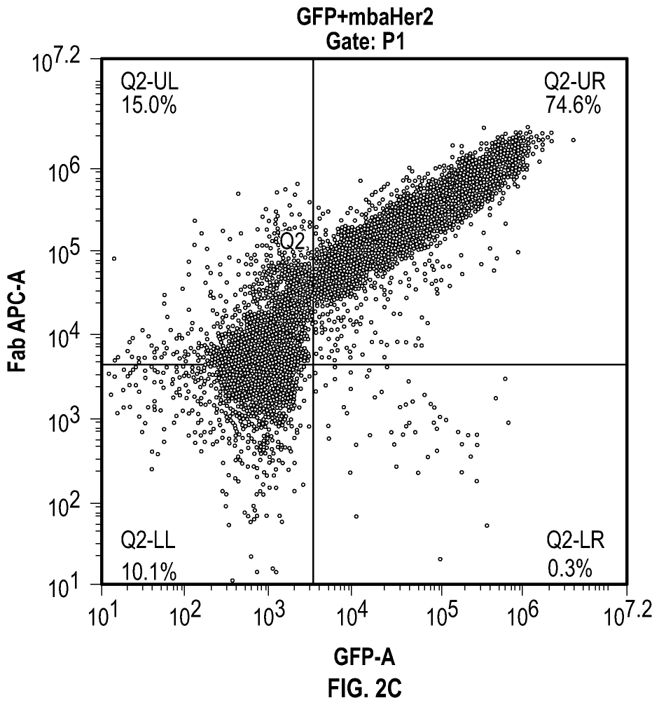

The ability of NK cells to effectively express this construct was first assessed. FIG. 2 depicts flow cytometry data related to the expression of mbaHer2 on the surface of expanded primary NK cells. Untransduced NK cells and mock-transduced NK cells (transduced with empty MSCV vector containing GFP only) were used as controls. The presence and relative abundance of the mbaHer2 was determined through staining the NK cells with allophycocyanin (APC) conjugated anti-Fab antibody. Green fluorescence protein (GFP) expression was used as an indicator of viral transduction. While both of the controls did not show mbaHer2 expression by flow cytometry analysis (FIGS. 2A-B), NK cells transduced with a vector containing anti-Her2 scFv and GFP showed robust mbaHer2 expression (FIG. 2C). Collectively, these data demonstrate that, in accordance with several embodiments disclosed herein, engineered adhesion constructs can successfully be expressed on NK cells.

In several embodiments, enhanced expression of the construct can be achieved by repeated transduction of the NK cells with a particular construct. In several embodiments, the components of the constructs can be delivered to a cell in a single vector, or alternatively using multiple vectors. Depending on the embodiment, the construct itself may lead to enhanced expression, for example a linear or head to tail construct may yield increased expression because of a lesser degree of in-cell assembly that a multiple subunit construct requires.

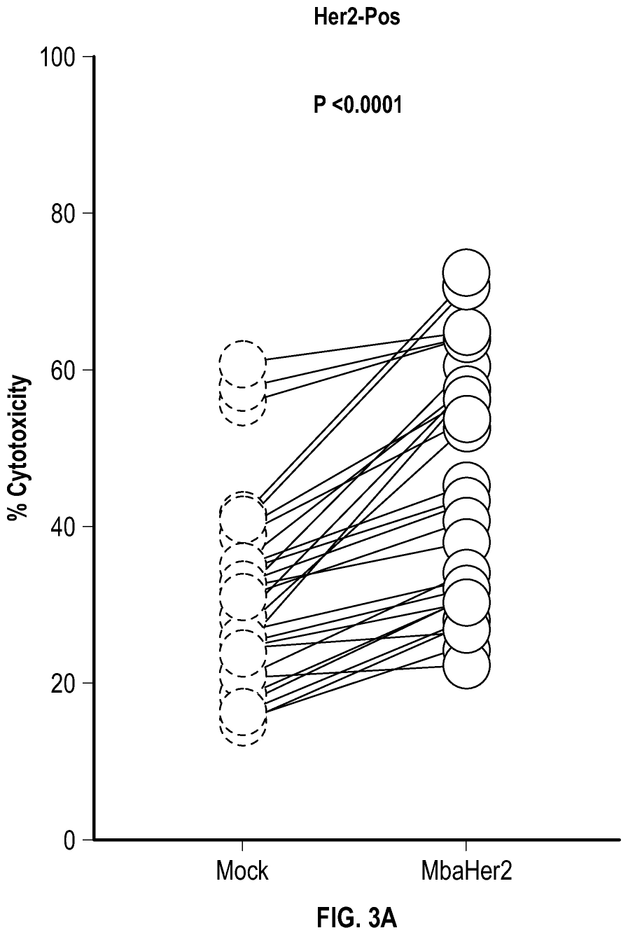

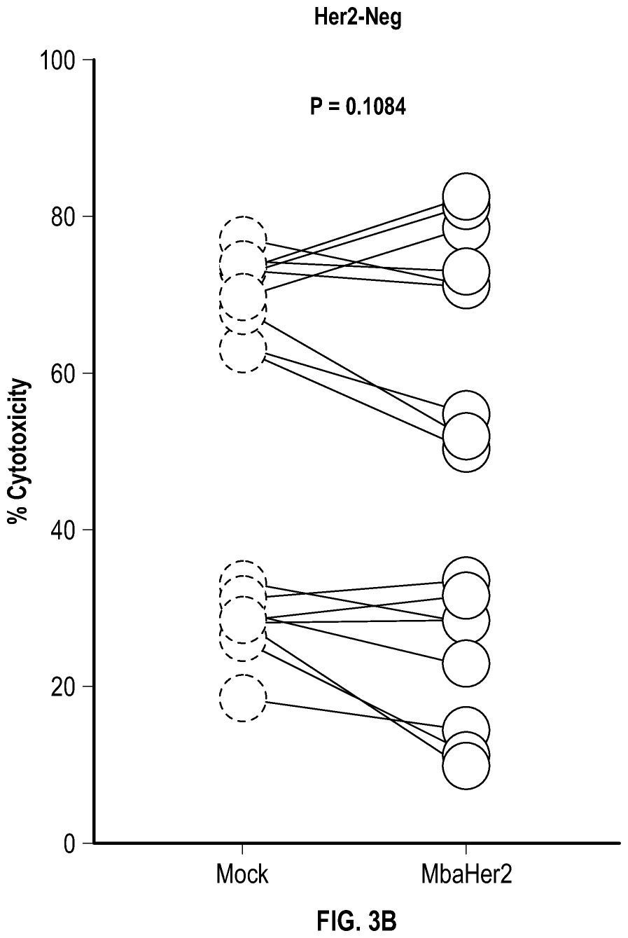

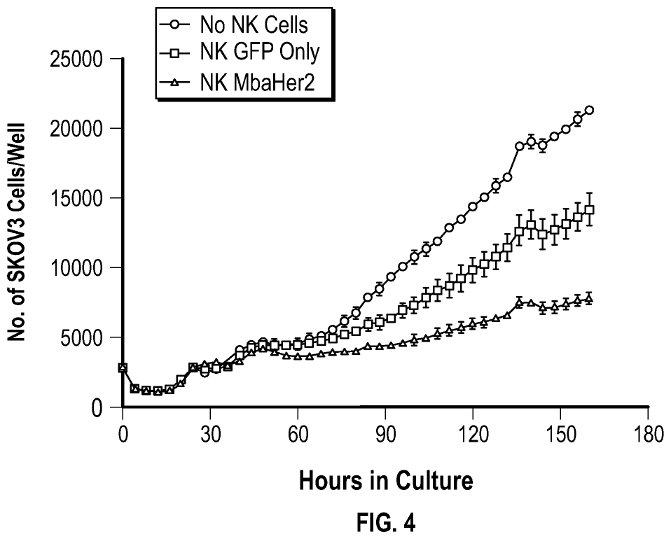

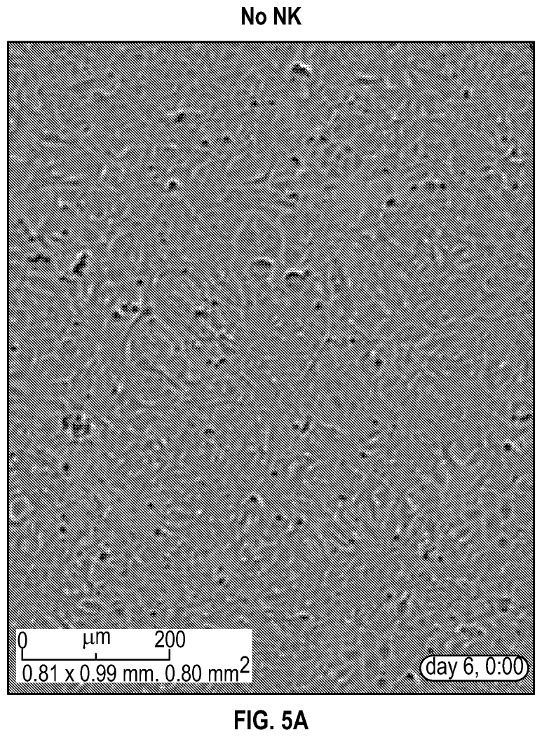

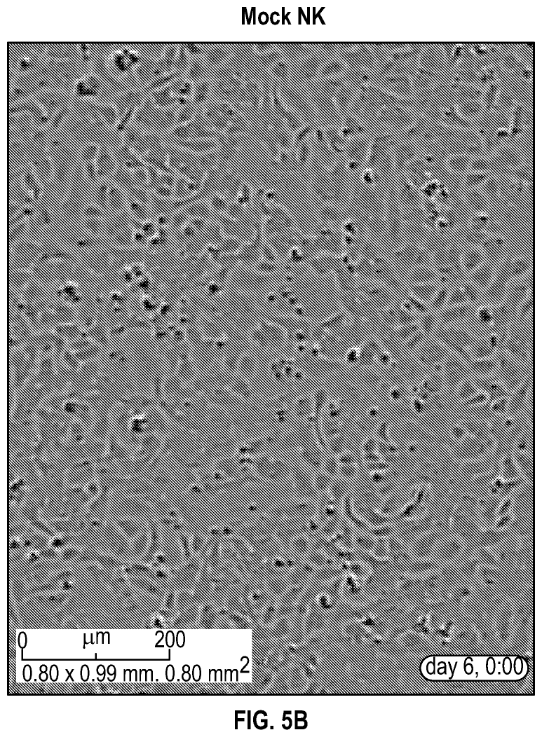

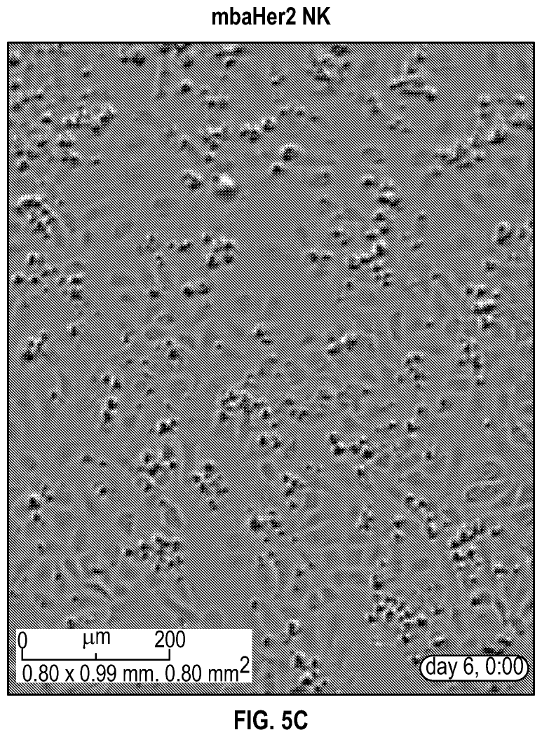

To evaluate the potency of the populations of transduced NK cells, cytotoxicity assays were performed using cancer cell lines that express high levels of Her2 (SKBR3, SKOV3, LNCap, ZR751) and low levels of Her2 (DU145, PLC/PRF/5). Consistent with the hypothesis that increased adhesion of mbaHer2-expressing NK cells to target cells via engagement of Her2 causes increased cytotoxicity, a significant increase in cytotoxicity was observed with mbaHer2-expressing NK cells against cancer cell lines expressing high levels of Her2 (FIG. 3A) but not cancer cells lines with low expression of Her2 (FIG. 3B). Additionally, NK cells expressing mbaHer2 displayed greater long-term cytotoxicity against SKOV3 cells relative to controls as measured by an IncuCyte live-imaging system (FIG. 4). Furthermore, and consistent with the cytotoxicity results described above, fewer numbers of mCherry-labelled SKOV3 cells were observed after 6 days of culture with NK cells expressing mbaHer2 relative to controls (FIG. 5). These data provide evidence that NK cells can not only be engineered to express adhesion receptor constructs, but those cells that express the adhesion receptors are able to be activated and successfully generate enhanced cytotoxic effects against target cells.

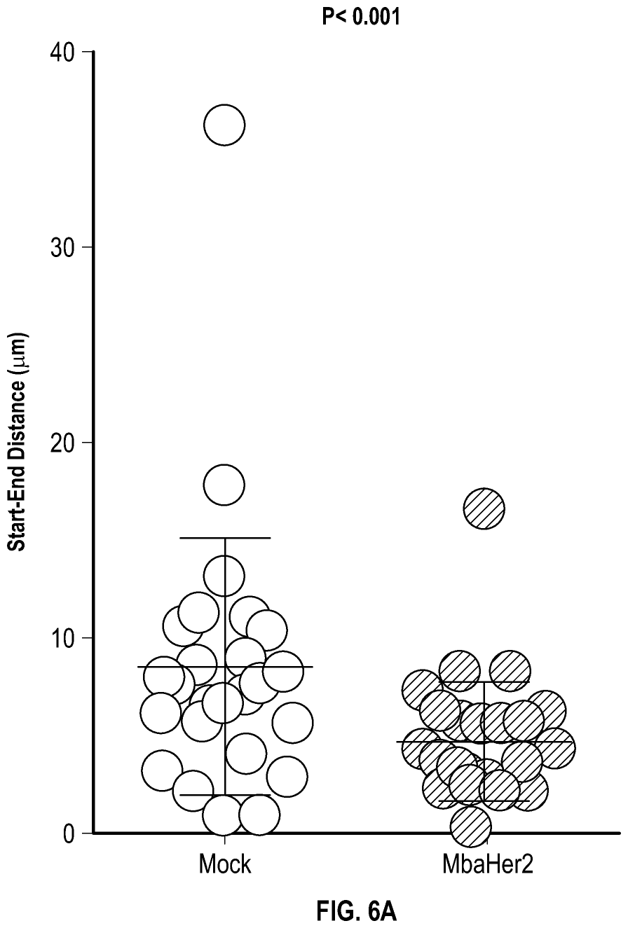

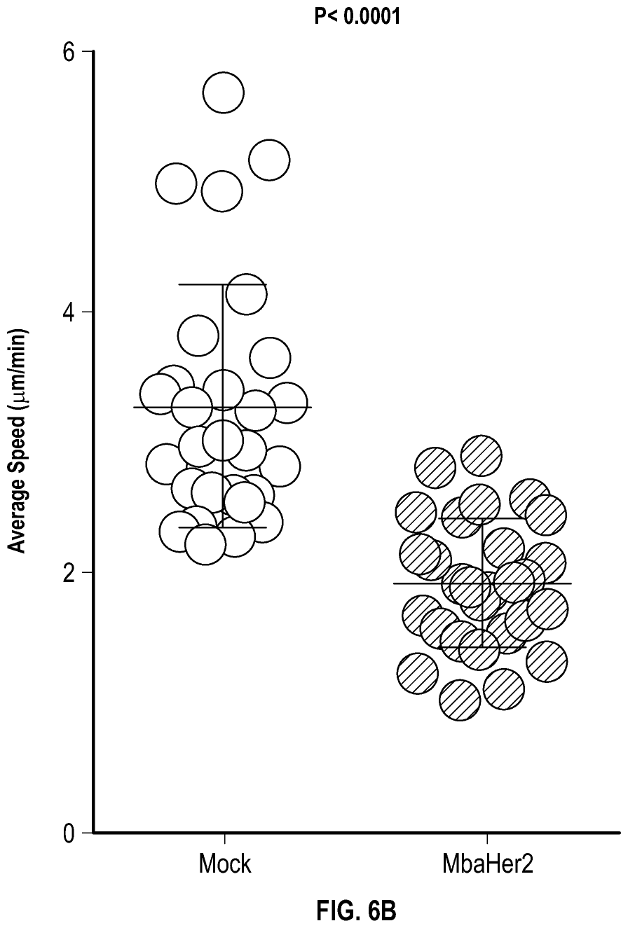

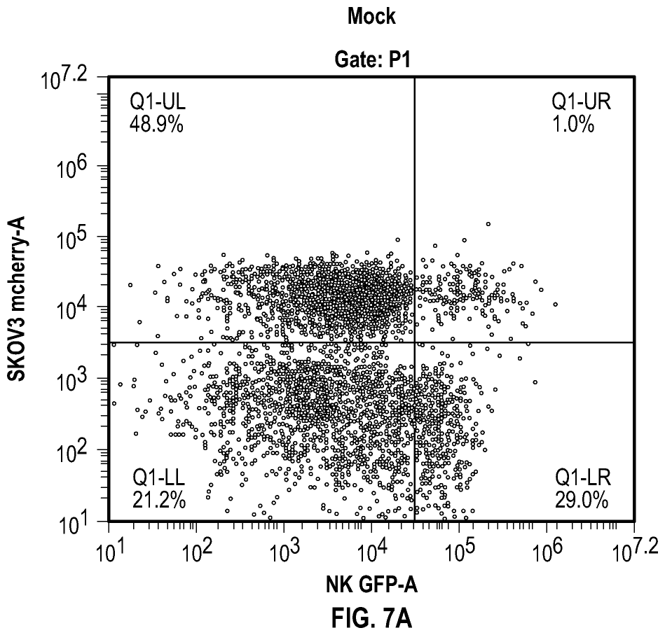

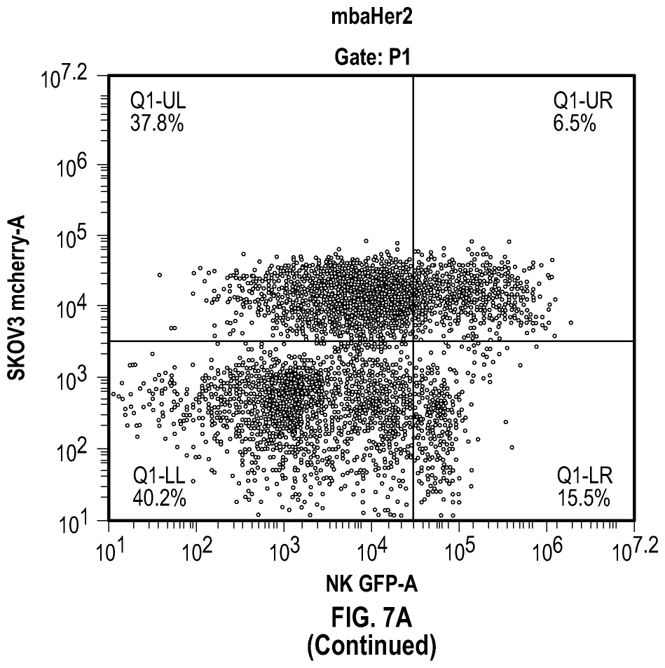

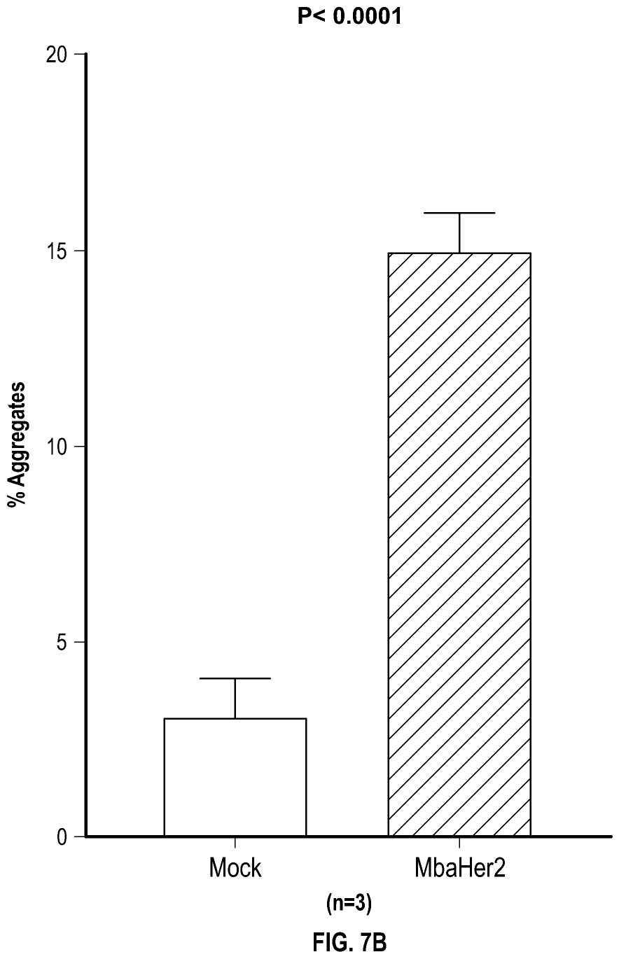

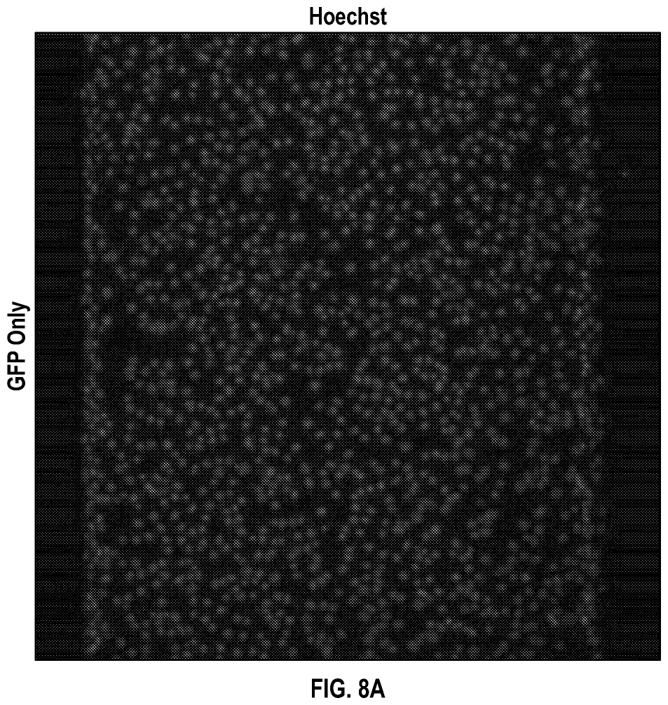

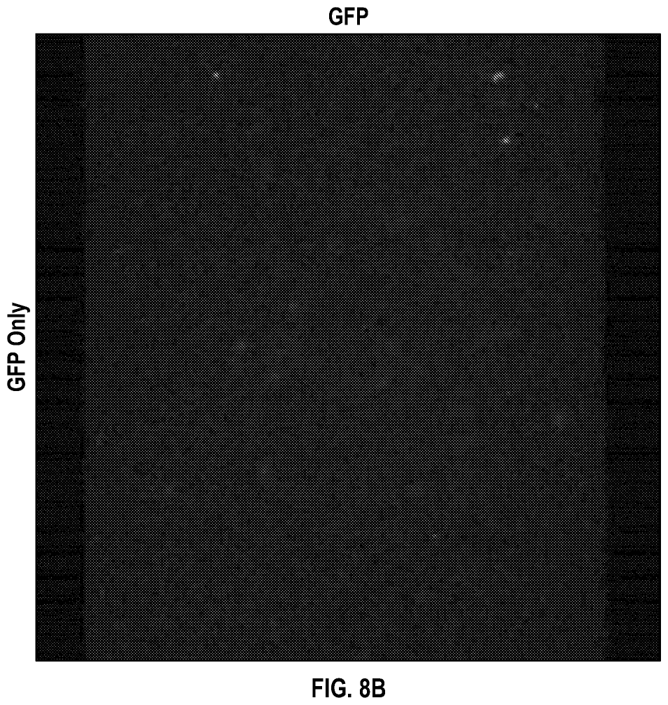

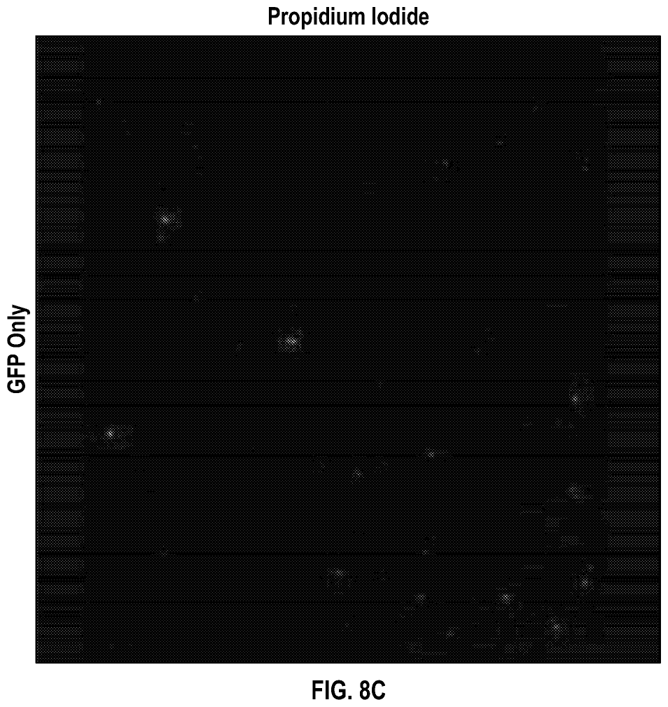

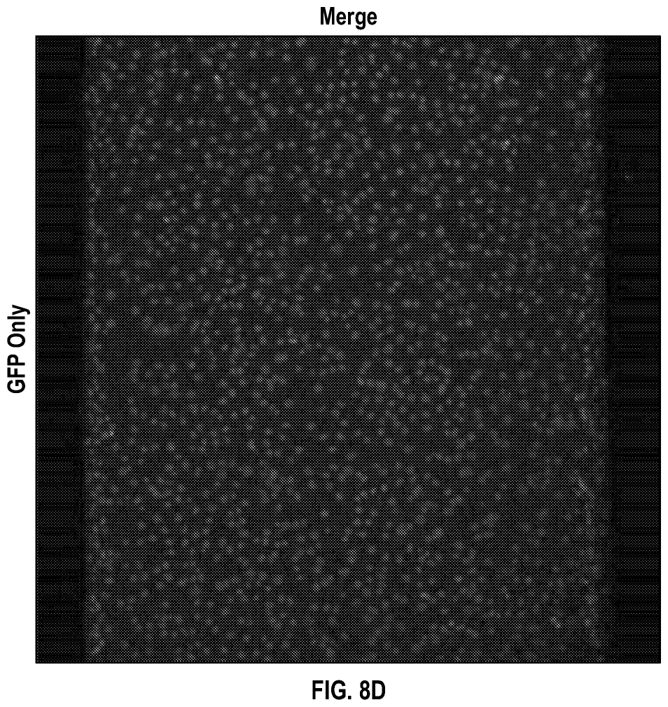

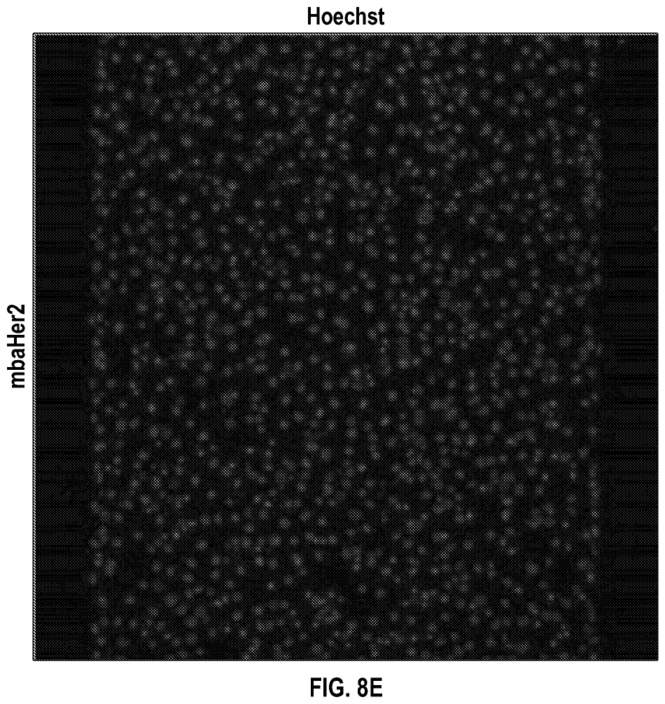

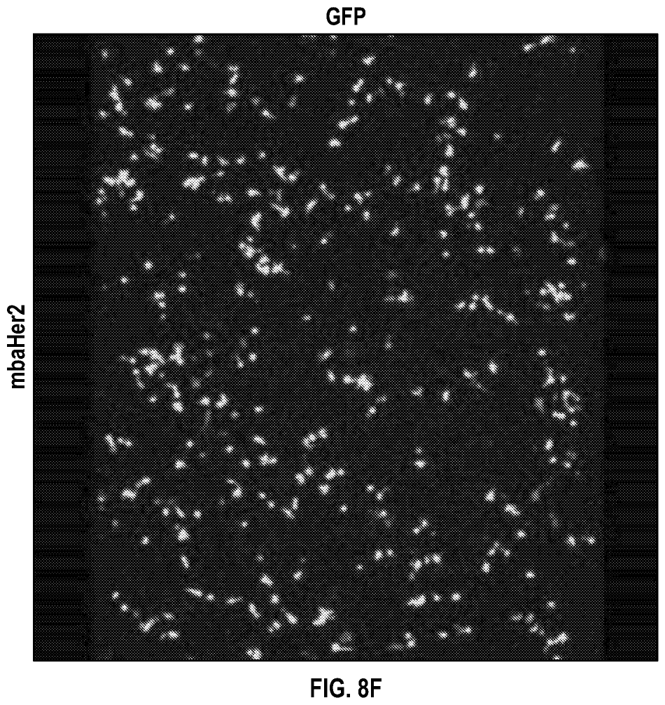

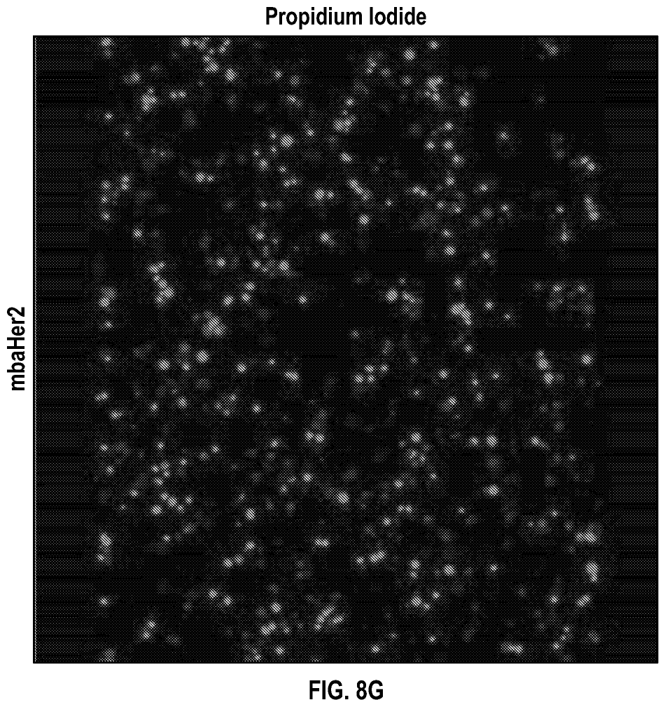

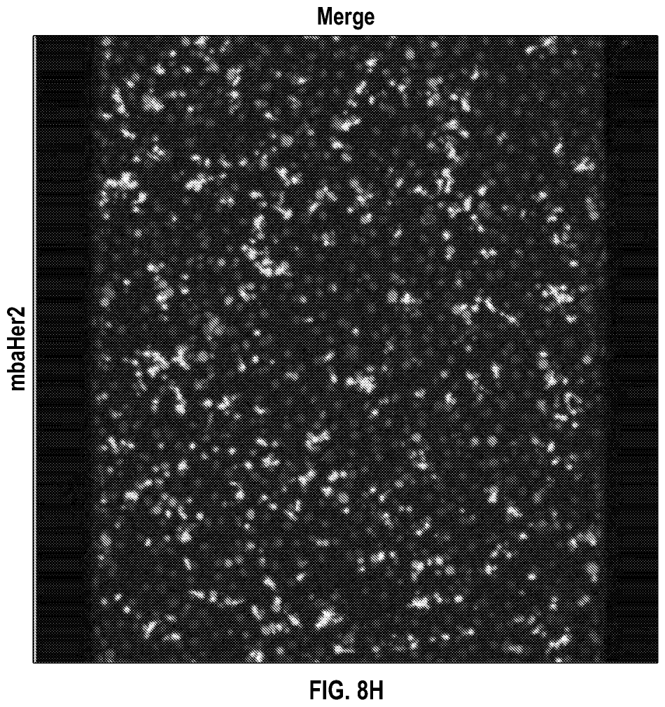

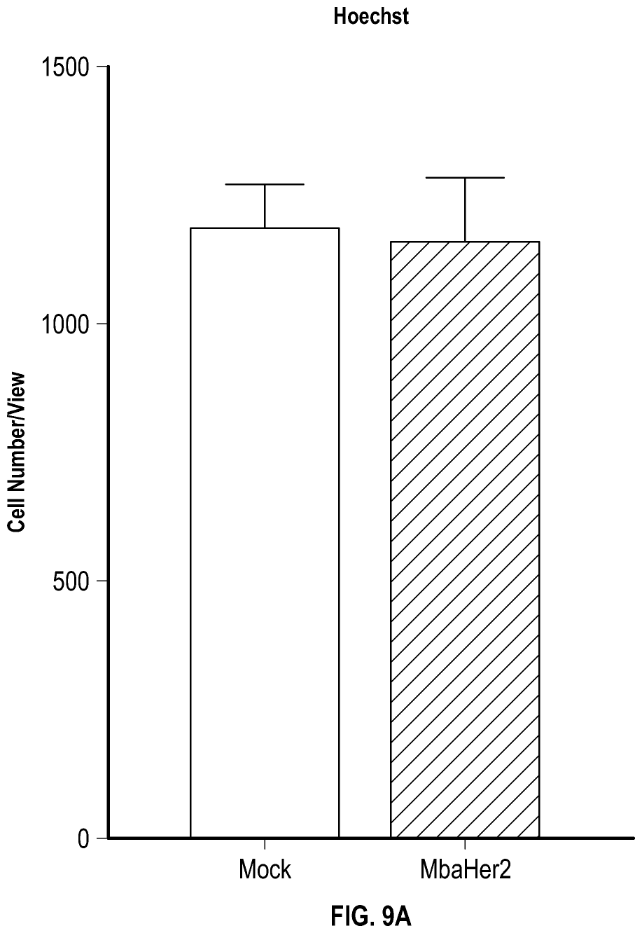

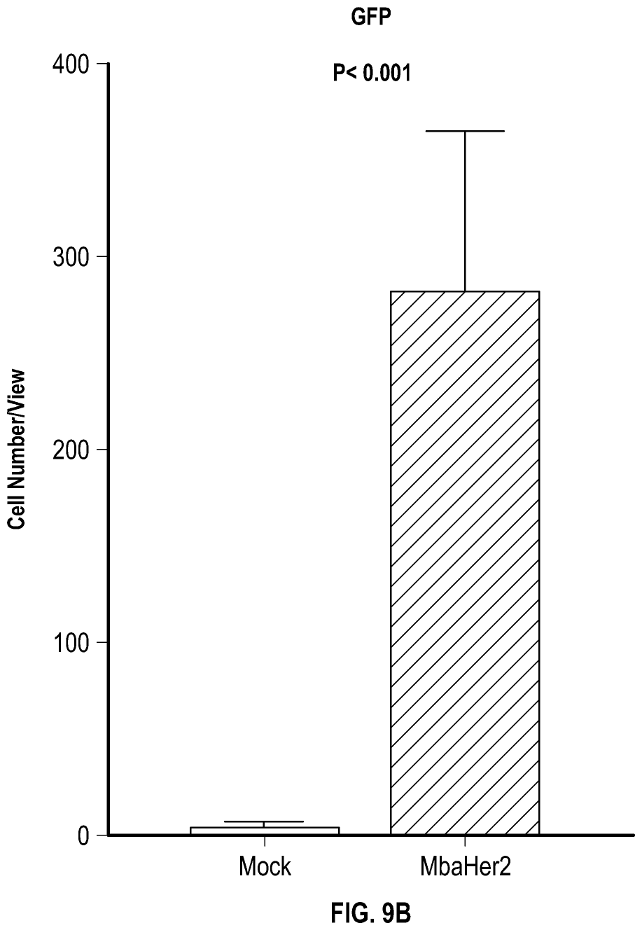

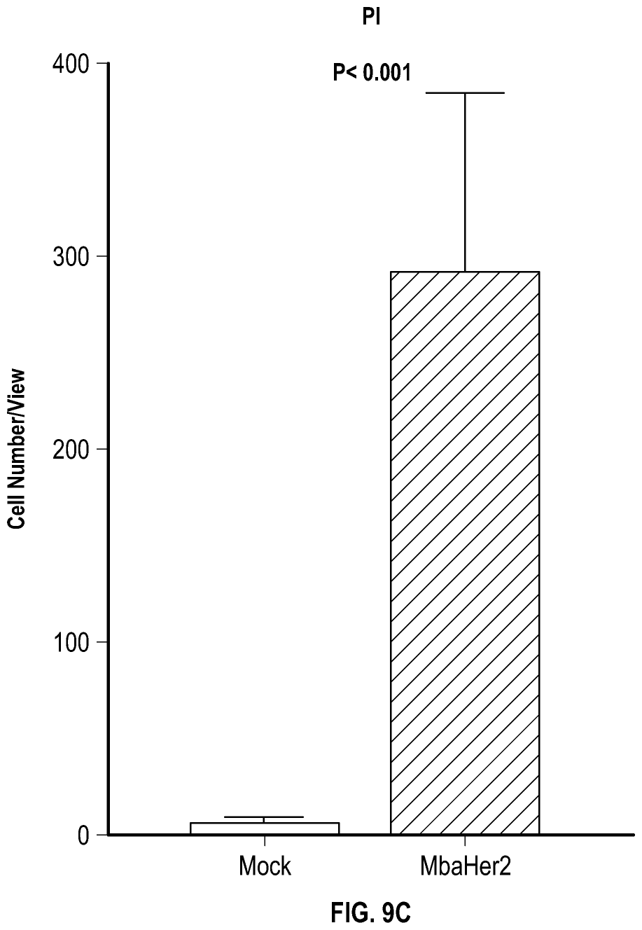

Further to the cytotoxicity data, the mechanism by which the NK cells are exerting these effects was examined, by evaluating the motility and aggregation of mock-transduced NK cells and NK cells expressing mbaHer2 seeded onto SKOV3 cells. Consistent with the cytotoxicity assays, NK cells expressing mbaHer2 displayed statistically significant reductions in distance traveled and average speed relative to the mock-transduced NK cells (FIG. 6). Furthermore, as shown in FIG. 7, NK cells expressing mbaHer2 had significantly increased aggregation with SKOV3 cells as measured flow cytometry. Importantly, these data indicate, according to some embodiments, that the adhesion constructs increase targeting of NK cells to target cells, reduce the time required for NK cells to engage target cells, and reduce the distance required for NK cells to travel to engage target cells. The adhesion of SKOV3 cells to the two NK cell populations was evaluated in a cell flow assay, where substantial co-localization of GFP (indicating NK expression of mbaHer2) and target cells staining positive for propidium iodide (indicating target cell death) was observed (FIG. 8). The quantification of the results of this assay (shown in FIG. 9) provides further evidence that expression of the adhesion receptor was responsible for target cell killing.

### Example 2—Anti-PSMA Adhesion Receptor Constructs

As disclosed herein, various adhesion receptor constructs comprising an extracellular receptor domain coupled with various transmembrane domains are provided. The present experiment was conducted to evaluate the expression and cytotoxic activity of an anti-PSMA adhesion receptor construct. The anti-PSMA adhesion receptor construct was prepared and tested according to the methods and materials described above. Depending on the construct, the methods used can be readily adjusted to account for variations required for generating, expressing and testing a construct.

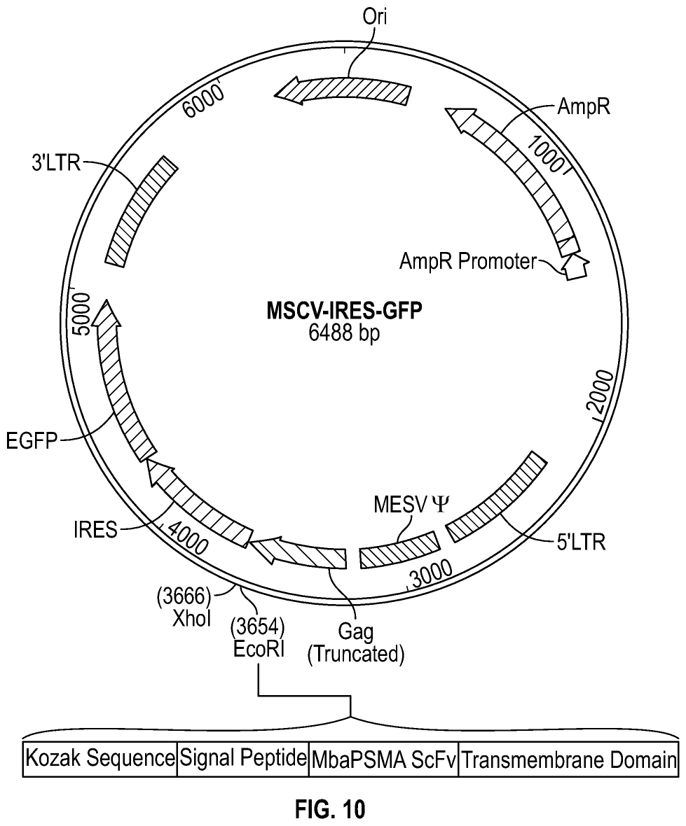

FIG. 10 depicts a plasmid map illustrating the point of insertion of membrane bound anti-PSMA (mbaPSMA) scFv into a Murine Stem Cell Virus (MSCV) plasmid. This plasmid map shows the insertion of a mbaPSMA construct into the EcoRI and XhoI restriction sites of the vector. The extracellular receptor domain of the mbaPSMA construct comprises an anti-PSMA single chain variable fragment (anti-PSMA scFv) and CD8 signal peptide that provides the desired membrane orientation of the receptor domain. The CD8 signal peptide comprises the amino acid sequence of SEQ ID NO. 68 and is encoded by the nucleic acid sequence of SEQ ID NO. 67. The anti-PSMA scFv comprises the amino acid sequence of SEQ ID NO. 63 and is encoded by the nucleic acid sequence of SEQ ID NO. 62. As shown in FIG. 10, the mbaPSMA construct further comprises a CD8 transmembrane domain downstream of the extracellular receptor domain that anchors the extracellular receptor domain on the surface of an NK cell. The CD8 transmembrane domain comprises the amino acid sequence of SEQ ID NO. 70 and is encoded by a nucleic acid of SEQ ID NO. 69. As shown in FIG. 10, the mbaPSMA construct further comprises a Kozak sequence of SEQ ID NO. 66 upstream of the signal peptide. Collectively, the mbaPSMA construct encodes an adhesion receptor that comprises the amino acid sequence of SEQ ID. 65 and is encoded by the nucleic acid sequence of SEQ ID NO. 64.

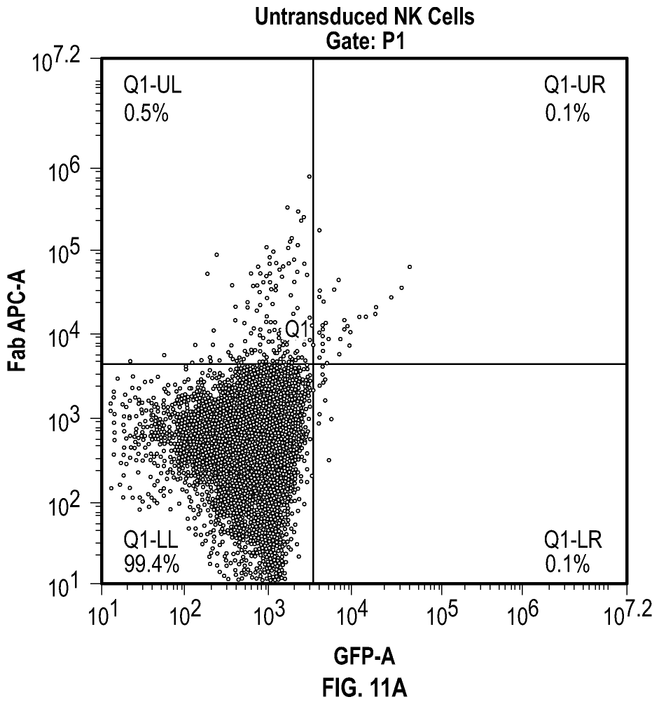

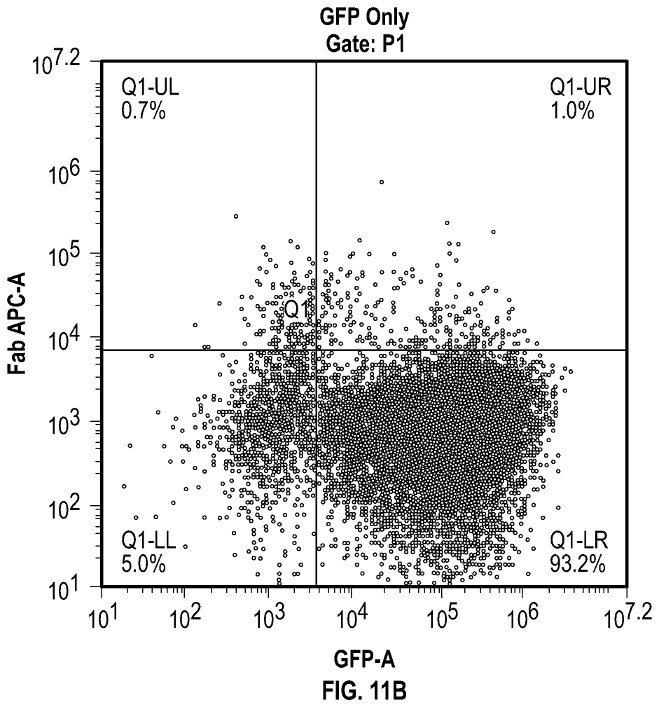

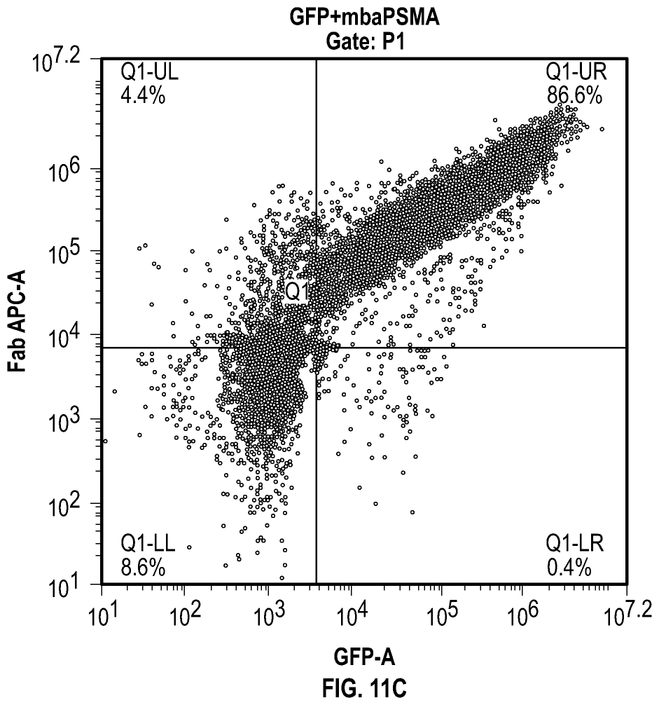

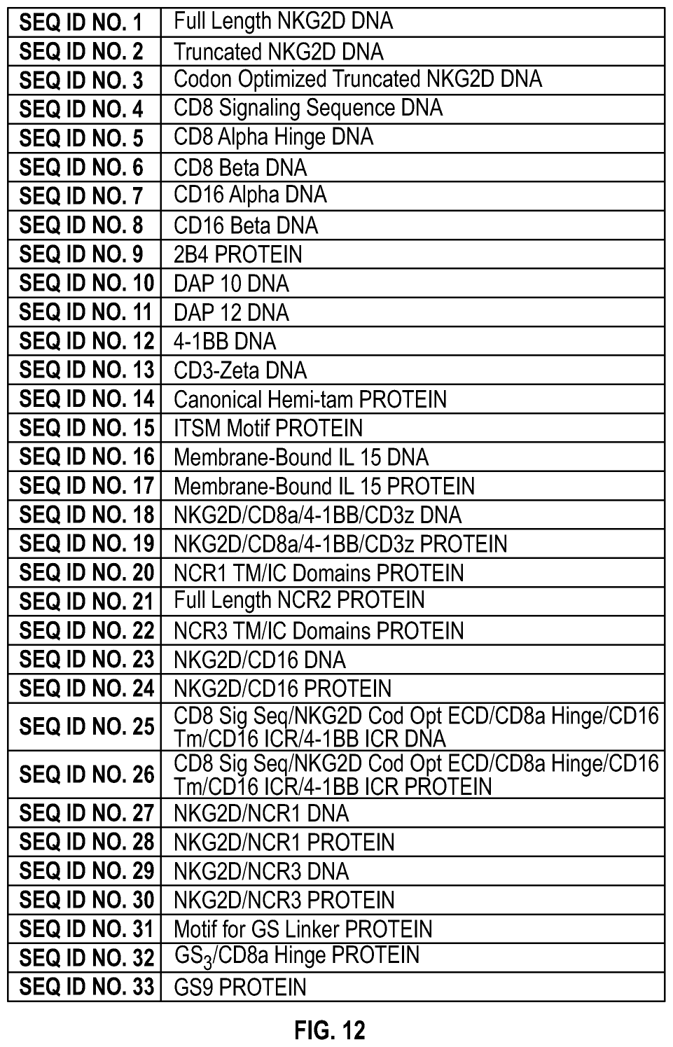

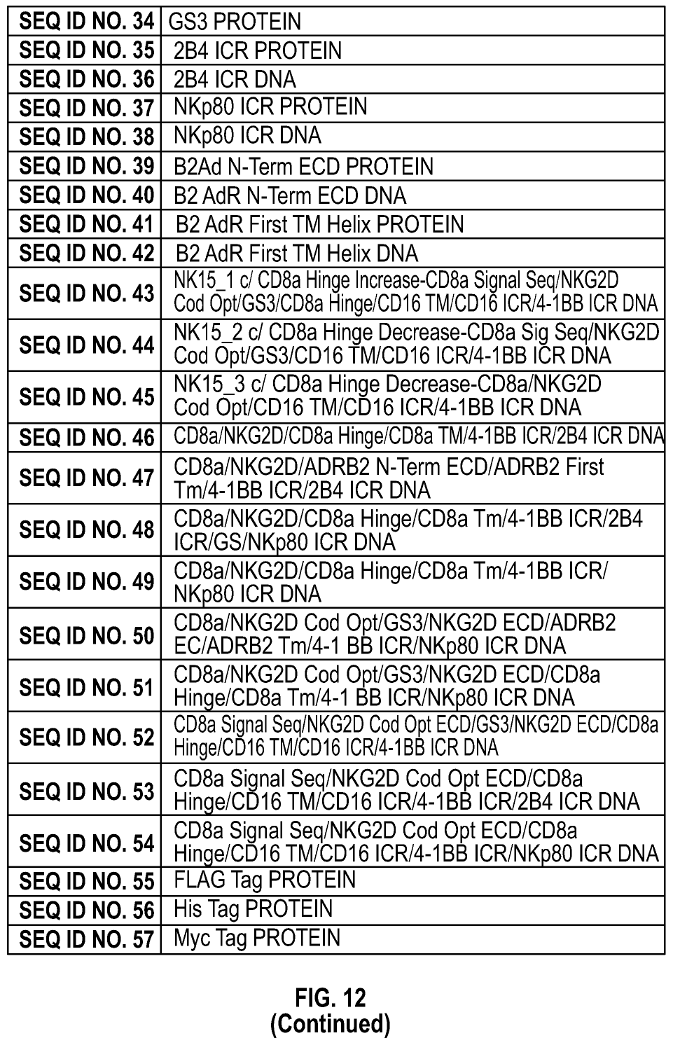

The ability of NK cells to effectively express this construct was next assessed. FIG. 11 depicts flow cytometry data related to the expression of mbaPSMA on the surface of expanded primary NK cells. Untransduced NK cells and mock-transduced NK cells (transduced with empty MSCV vector containing GFP only) were used as controls. The presence and relative abundance of the mbaPSMA was determined through staining the NK cells with allophycocyanin (APC) conjugated anti-Fab antibody. Green fluorescence protein (GFP) expression was used as an indicator of viral transduction. While both of the controls did not show mbaPSMA expression by flow cytometry analysis (FIGS. 11A-B), NK cells transduced with a vector containing anti-PSMA scFv and GFP showed robust mbaPSMA expression (FIG. 11C). Collectively, these data demonstrate that, in accordance with several embodiments disclosed herein, engineered adhesion constructs can successfully be expressed on NK cells.

### Example 3—In Vitro Assessment of Anti-PSMA-NK Construct

Further experiments were undertaken to evaluate the long-term cytotoxicity of NK constructs according to several embodiments disclosed herein. In particular, this experiment was designed to evaluate the in vitro cytotoxicity of an NK construct comprising an anti-PSMA adhesion receptor. A prostate cancer cell line, DU145 cells, was used as the target cell population. DU145 cells are PSMA negative in their native state. Here, however, DU145 cells were transduced such that they expressed PSMA (“DU145-PSMA”).

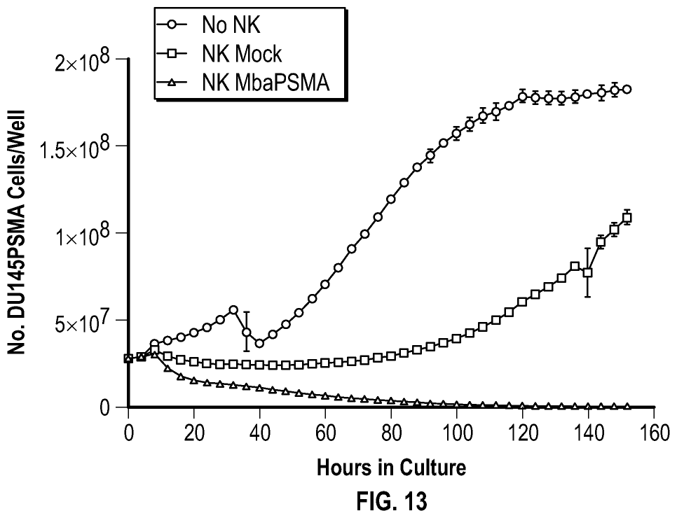

DU145-PSMA were plated and NK cells comprising a membrane-bound anti-PSMA adhesion receptor were added 24 hours later. NK cells were added at a 1:1 effector:target cell ratio. DU145-PSMA cell number per well was evaluated over time, with data collected out to 150 hours post-NK cell addition. Data were collected for control (no NK cells added), NK Mock (native NK cells without an adhesion receptor, but expressing GFP) and NK MbaPSMA (NK cells engineered to express an anti-PSMA adhesion receptor). Mean of triplicate measurements are shown in FIG. 13. Cell viability was assessed by the IncuCyte live-imaging system (Essen).

As shown in FIG. 13, DU145-PSMA cells that were not exposed to NK cells proliferated throughout the duration of the experiment reaching a plateau of approximately 1.75×108 cells per well after approximately 120 hours in culture (“No NK” curve in FIG. 13). DU145-PSMA exposed to native NK cells (“NK Mock” curve in FIG. 13) also exhibited proliferation throughout the experiment, albeit at a slower rate as compared to control, and also reaching a lower overall population number. In contrast, DU145-PSMA co-cultured with NK cells expressing an anti-PSMA adhesion receptor exhibited very limited proliferation, which is primarily in the first few hours of the co-culture (“NK MbaPSMA” curve in FIG. 13). After approximately 8 to 10 hours, DU145-PSMA cell numbers began to decline, with the number of cells per well reaching zero after approximately 100 hours in culture. These data demonstrate that, according to several embodiments, engineering NK cells to express an adhesion receptor in order to more efficiently localize the NK cells at a target cell population, result in enhanced long-term cytotoxicity. Depending on the embodiment, enhanced cytotoxicity is exhibited for a duration of about 50 hours, about 75 hours, about 100 hours, about 125 hours, about 150 hours, or longer. In several embodiments, the enhanced cytotoxicity results in a reduced number of doses of an NK cell immunotherapy construct being required to treat a patient (e.g., fewer doses than required to treat a patient with NK cells that do not express an adhesion receptor as disclosed herein).

In particular, it is notable that the long-term cytotoxicity is enhanced over that of native NK cells, which are known to naturally exhibit a relatively high degree of cytotoxicity on their own. As with this non-limiting example, according to several embodiments, an anti-PSMA scFv is used, such as that described in the prior example (e.g., an anti-PSMA scFv comprising the amino acid sequence of SEQ ID NO. 63 and encoded by the nucleic acid sequence of SEQ ID NO. 62). However, as described herein, other adhesion receptors may also be used in order to target, and thus enhance NK based cytotoxicity against, other target cell populations. In several embodiments, the use of an adhesion receptor is beneficial to enhance the efficacy of an NK based cancer immunotherapy regime.

### Example 4—Further In Vitro Assessment of Additional Anti-PSMA-NK Construct

To further demonstrate the ability of adhesion constructs to enhance the cytotoxicity of NK cells against various target cells, an additional in vitro evaluation of the long-term cytotoxicity of NK constructs according to several embodiments disclosed herein was assessed. This experiment was designed to evaluate the in vitro cytotoxicity of an NK construct comprising an anti-PSMA adhesion receptor against the androgen-dependent LNCap prostate cancer line, which expresses PSMA endogenously.

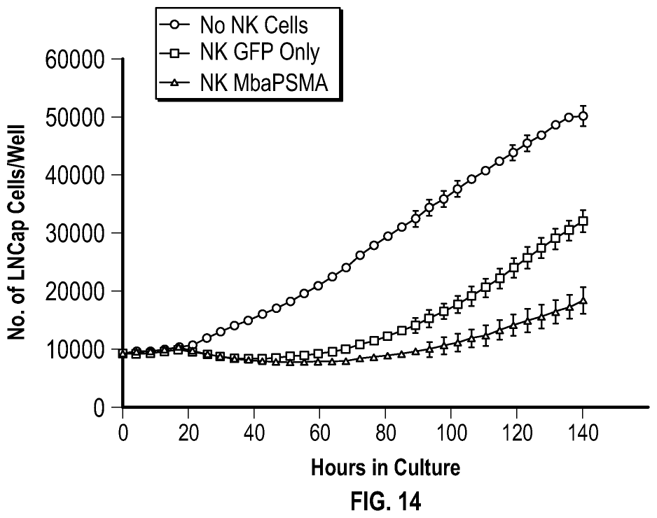

LNCap cells were plated and NK cells comprising a membrane-bound anti-PSMA adhesion receptor were added 24 hours later. NK cells were added at a 1:1 effector:target cell ratio. LNCap cell number per well was evaluated over time, with data collected out to 130 hours post-NK cell addition. Data were collected for control (no NK cells added), NK Mock (native NK cells without an adhesion receptor, but expressing GFP) and NK MbaPSMA (NK cells engineered to express an anti-PSMA adhesion receptor). Mean of triplicate measurements are shown in FIG. 14. Cell viability was assessed by the IncuCyte live-imaging system (Essen).

As shown in FIG. 14, LNCap cells that were not exposed to NK cells proliferated throughout the duration of the experiment reaching approximately 50,000 cells per well after 130 hours in culture (“No NK Cells” curve in FIG. 14). LNCap cells exposed to native NK cells (“NK GFP only” curve in FIG. 14) also exhibited proliferation throughout the experiment, albeit at a slower rate as compared to control, and also reaching a lower overall population number (30,000 cells per well at 130 hours). LNCap cells co-cultured with NK cells expressing an anti-PSMA adhesion receptor exhibited limited proliferation over the duration of the experiment (“NK MbaPSMA” curve in FIG. 14). Cell numbers remained relatively constant (at 10,000 cells per well) for the first 18-20 hours of co-culture. Thereafter, LNCap cell number dropped and was below ˜10,000 cells per well from hours 20-90 of co-culture. At approximately hour 100, LNCap cell number had returned to baseline and a modest increase was detected from approximately hours 100-120. While there was a slight overall increase in LNCap cells number, there is a clear reduction in proliferation as compared to the control and native NK groups. Thus, these demonstrate that, according to several embodiments, engineering NK cells to express an adhesion receptor in order to more efficiently localize the NK cells at a target cell population, result in enhanced long-term cytotoxicity against the target cell population. Depending on the embodiment, enhanced cytotoxicity is exhibited for a duration of about 10-20 hours, about 20-30 hours, about 30-40 hours, about 40-50 hours, or longer. In several embodiments, the enhanced cytotoxicity results in a reduced number of doses of an NK cell immunotherapy construct being required to treat a patient (e.g., fewer doses than required to treat a patient with NK cells that do not express an adhesion receptor as disclosed herein), in particular a patient with androgen-dependent prostate cancer.

As with the prior example, it is notable that the long-term cytotoxicity is enhanced over that of native NK cells, which are known to naturally exhibit a relatively high degree of cytotoxicity on their own. According to several embodiments, an anti-PSMA scFv is used, such as that described herein. However, other adhesion receptors may also be used to target, and thus enhance NK based cytotoxicity against, other target cell populations. In several embodiments, the use of an adhesion receptor is beneficial to enhance the efficacy of an NK based cancer immunotherapy regime.

### Example 5 In Vivo Assessment of Anti-HER2-NK Construct

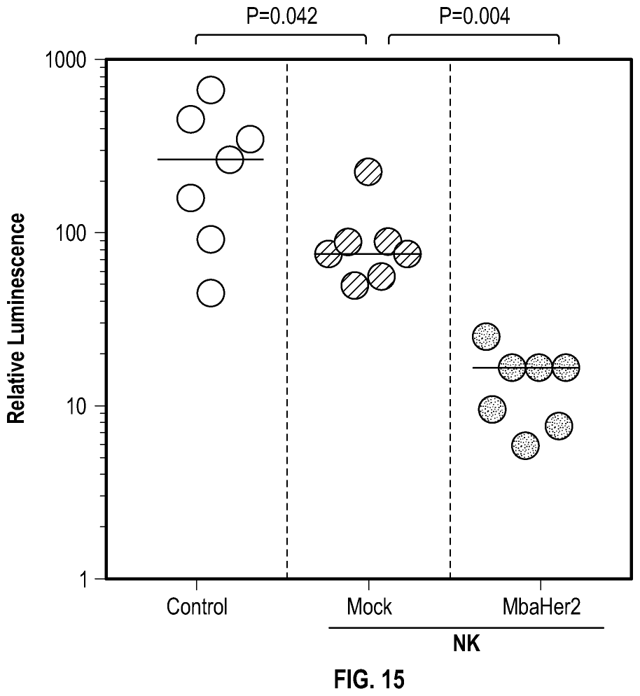

Building on the in vitro examples discussed herein, an additional non-limiting experiment was conducted to evaluate the effects of targeting NK cells using adhesion receptors as disclosed herein on in vivo cytotoxicity against a target cell population. In this non-limiting example, the HER2 expressing ovarian carcinoma line SKOV-3 was used as the target cell population. SKOV-3 cells were transduced such that they expressed firefly luciferase. The transduced SKOV-3 cells were injected intraperitoneally into 21 NOD.Cg-Prkdc^{SCID} IL2rg^{tm1Wj1}/SzJ (NOD/scid IL2RGnull) mice at a dose of 0.2×10⁶ cells per mouse. Three experimental groups were set up (7 mice per group), as follows: (i) control, which would not receive NK cells but was injected with tissue culture medium, (ii) Mock, which would receive NK cells transduced with GFP only, and (iii) Mba-Her2, which would receive NK cells transduced with a membrane bound anti-Her2 adhesion receptor. NK cells were injected intraperitoneally 3 and 7 days after the injection of SKOV-3 cells. NK cells doses for each injection were 1×10⁷ cells per mouse. Each mouse also received intraperitoneal injections of IL-2 (20,000 IU each injection) three times per week. Ventral and dorsal tumor luminescence was measured with the Xenogen IVIS-200 system (Caliper Life Sciences) Baseline tumor luminescence was recorded prior to SKOV-3 cell injection and at 22 days after injection. Luminescent imaging began five minutes after an intraperitoneal injection of an aqueous solution of D-luciferin potassium salt (3 mg/mouse, Perkin Elmer). Photons were quantified using the Living Image 3.0 software program. Data is depicted in FIG. 15.

The left column of FIG. 15 shows data for the control mice. Relative luminescence indicates an increased tumor burden in these mice 22 days after injection of SKOV-3 ovarian cancer cells. In contrast, the central panel (administration of the NK cells expressing GFP only) depicts data that represents a significantly reduced tumor burden as compared to control (represented by a reduced relative luminescence). The right panel of FIG. 15 shows data for the group of mice receiving NK cells expressing an anti-Her2 adhesion construct. Relative luminescence in this group of mice was lower than that of both the Mark and control groups, demonstrating a significant reduction in tumor burden (p value equals 0.004 as compared to mock). Statistical analysis in FIG. 15 is by t test. As discussed above in connection with the in vitro experiments, the in vivo data shows an unexpected increase in the cytotoxicity of NK cells expressing an adhesion receptor, especially when considering the relatively high natural cytotoxicity of native NK cells. These data corroborate the in vitro studies in that the expression of an adhesion receptor construct facilitates enhanced antitumor activity of NK cells. The expression of the adhesion receptor may enhance the homing of the NK cells to target cells bearing a target antigen, according to several embodiments. In addition, in accordance with several embodiments, the expression of an adhesion receptor may allow a higher affinity interaction to occur between the NK cell in the target cell. Likewise, in several embodiments the expression of an adhesion receptor may increase the duration of residency (e.g. the time span over which an NK cell interacts with a target cell) of an NK cell on a target cell. In several embodiments the expression of an adhesion receptor has one or more of such effects, which can lead to an overall increased efficacy of an NK based immunotherapy regime. In some embodiments, the expression of an adhesion receptor reduces one or more of the dose, frequency of administration, or duration of overall cancer immunotherapy treatment. In several embodiments expression of an adhesion receptor can improve patient survival rates for patients receiving NK based immunotherapy treatment. All such comparisons recited above (or elsewhere herein) described a comparison of a characteristic of the NK cells expressing adhesion receptors as disclosed herein compared to NK cells that do not express such an adhesion receptor.

It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. For example, actions such as “administering a population of expanded NK cells” include “instructing the administration of a population of expanded NK cells.” In addition, where features or aspects of the disclosure are described in terms of Markush groups, those skilled in the art will recognize that the disclosure is also thereby described in terms of any individual member or subgroup of members of the Markush group.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers. For example, “about 10 nanometers” includes “10 nanometers.”

1.-53. (canceled)

54. A polynucleotide encoding an adhesion receptor, the adhesion receptor comprising:
(a) an extracellular receptor domain,
wherein said extracellular receptor domain comprises a peptide that binds a target cell antigen;
wherein the target cell antigen is differentially expressed between healthy cells and target cells;
wherein the target cells are targeted for recognition and destruction by Natural Killer (NK) cells;
wherein the peptide that binds a target cell antigen comprises a single-chain Fv (scFv) or a fragment thereof; and
(b) a transmembrane domain, wherein the transmembrane domain anchors the extracellular receptor domain on the surface of the NK cells,
wherein the polynucleotide is co-expressed with an additional construct encoding membrane-bound interleukin 15 (mbIL15).

55. The polynucleotide of claim 54, wherein the target cell antigen is associated with a disease.

56. The polynucleotide of claim 55, wherein the disease is a cancer.

57. The polynucleotide of claim 55, wherein the disease is a viral, bacterial, fungal and/or parasitic infection.

58. The polynucleotide of claim 54, wherein the target cell antigen comprises one or more of Fms-Like Tyrosine Kinase 3 (FLT3), Syndecan 1, epidermal growth factor receptor (EGFR), CD19, CD70, BCMA, Her2, PSMA, and CD123.

59. The polynucleotide of claim 54, wherein NK cells that express the adhesion receptor encoded by the polynucleotide: (i) bind target cells more rapidly as compared to NK cells that do not express the adhesion receptor, (ii) have enhanced homing to tumors or infected sites as compared to NK cells that do not express the adhesion receptor, (iii) show enhanced cytotoxic activity against cells presenting target cell antigens as compared to NK cells that do not express the adhesion receptor, and/or (iv) have reduced off target cytotoxic effects as compared to NK cells that do not express the adhesion receptor.

60. The polynucleotide of claim 54, wherein the polynucleotide further encodes a chimeric receptor comprising:
(a) an extracellular receptor domain, wherein said extracellular receptor domain comprises a peptide that binds native ligands of Natural Killer Group 2 member D (NKG2D); and
(b) an effector domain comprising a transmembrane region and an intracellular signaling domain, and
wherein the polynucleotide further encodes membrane-bound interleukin 15 (mbIL15).

61. The polynucleotide of claim 54, wherein the adhesion receptor comprises an anti-Her2 scFv, and wherein the scFv is encoded by the nucleic acid sequence of SEQ ID NO: 58, SEQ ID NO: 60 or a nucleic acid sequence at least about 95% homologous to SEQ ID NO: 58 or SEQ ID NO: 60.

62. The polynucleotide of claim 61, wherein the scFv comprises the amino acid sequence of SEQ ID NO: 59, SEQ ID NO: 61, or an amino acid sequence at least about 95% homologous to SEQ ID NO: 59 or SEQ ID NO: 61.

63. The polynucleotide of claim 54, wherein the adhesion receptor comprises an anti-PSMA scFv, wherein the scFv is encoded by the nucleic acid sequence of SEQ ID NO: 62, SEQ ID NO: 64 or a nucleic acid sequence at least about 95% homologous to SEQ ID NO: 62 or SEQ ID NO: 64.

64. The polynucleotide of claim 63, wherein the scFv comprises the amino acid sequence of SEQ ID NO: 63, SEQ ID NO: 65, or an amino acid sequence at least about 95% homologous to SEQ ID NO: 63 or SEQ ID NO: 65.

65. A polynucleotide encoding an adhesion receptor, the adhesion receptor comprising:
(a) an extracellular receptor domain,
wherein said extracellular receptor domain comprises a peptide that binds a target cell antigen expressed by a cell, wherein the target cell antigen is associated with cancer;
wherein the target cell antigen is differentially expressed between healthy cells and target cells;
wherein the peptide that binds the target cell antigen is an antibody or an antibody fragment; and
(b) a transmembrane domain, wherein the transmembrane domain anchors the extracellular receptor domain on the surface of the NK cell, and
wherein the polynucleotide further encodes membrane-bound interleukin 15 (mbIL15).

66. The polynucleotide of claim 65, wherein the mbIL15 is encoded by a sequence having at least about 95% homology to SEQ ID NO: 16.

67. The polynucleotide of claim 65, wherein the extracellular receptor domain binds CD19.

68. The polynucleotide of claim 65, wherein the further encodes a chimeric receptor that comprises an extracellular receptor domain comprising a peptide that binds native ligands of Natural Killer Group 2 member D (NKG2D).

69. The polynucleotide of claim 65, wherein the extracellular receptor domain further comprises a second peptide that binds an additional target cell antigen that is different than the target cell antigen.

70. A polynucleotide encoding an adhesion receptor, the adhesion receptor comprising:
(a) an extracellular receptor domain,
wherein said extracellular receptor domain comprises a peptide that binds a target cell antigen expressed by a cell, wherein the target cell antigen is associated with cancer;
wherein the target cell antigen is differentially expressed between healthy cells and target cells;
wherein the peptide that binds the target cell antigen is an antibody or an antibody fragment; and
(b) a transmembrane domain, wherein the transmembrane domain anchors the extracellular receptor domain on the surface of the NK cell,
wherein the polynucleotide further encodes a chimeric receptor comprising:
an additional extracellular receptor domain, wherein said extracellular receptor domain comprises a peptide that binds native ligands of Natural Killer Group 2 member D (NKG2D); and
an additional effector domain comprising a transmembrane region and an intracellular signaling domain.

71. The polynucleotide of claim 70, wherein the polynucleotide further encodes membrane-bound interleukin 15 (mbIL15).

72. The polynucleotide of claim 71, wherein the mbIL15 is encoded by a sequence having at least about 95% homology to SEQ ID NO: 16.

73. The polynucleotide of claim 70, wherein the target cell antigen comprises one or more of Fms-Like Tyrosine Kinase 3 (FLT3), Syndecan 1, epidermal growth factor receptor (EGFR), CD19, CD70, BCMA, Her2, PSMA, and CD123.
